(19) United States
(12) Patent Application Publication
(10) Pub. No.: US 2013/0330294 A1
(43) Pub. Date: Dec. 12, 2013

(54) Serum Amyloid A (SAA) Overrides Regulatory T Cells (TREG) Anergy

(71) Applicants: Elizabeth D. Mellins, Stanford, CA (US); Khoa D. Nguyen, San Francisco, CA (US)

(72) Inventors: Elizabeth D. Mellins, Stanford, CA (US); Khoa D. Nguyen, San Francisco, CA (US)

(73) Assignee: The Board of Trustees of the Leland Stanford Junior University, Palo Alto, CA (US)

(21) Appl. No.: 13/881,912

(22) PCT Filed: Nov. 7, 2011
(86) PCT No.: PCT/US11/59598

§ 371 Date: Jul. 16, 2013

Related U.S. Application Data
(60) Provisional application No. 61/411,461, filed on Nov. 8, 2010.

(51) Int. Cl. C07K 14/775; C12N 5/0783; A61K 38/17; A61K 35/14; G01N 33/92; A61K 45/06

(52) U.S. Cl. CPC C07K 14/775; G01N 33/92; A61K 45/06; A61K 38/1709; A61K 35/17; C12N 5/0637
USPC 424/85.2; 435/375; 435/7.24; 424/278.1; 424/93.71

(57) ABSTRACT

Methods and compositions are provided for inducing the proliferation of regulatory T cells. These methods find a number of uses, including, for example, treating autoimmune and rheumatoid diseases. Also provided are reagents and kits that find use in these methods.

### CROSS REFERENCE TO RELATED APPLICATIONS

Pursuant to 35 U.S.C. §119 (e), this application claims priority to the filing date of the U.S. Provisional Patent Application Ser. No. 61/411,461 filed Nov. 8, 2010; the disclosure of which is herein incorporated by reference.

### STATEMENT REGARDING GOVERNMENT SUPPORT

This invention was made with Government support under contract R21AI075254-01A1 awarded by National Institute of Allergies and Infectious Disease. The Government has certain rights in this invention.

### BACKGROUND OF THE INVENTION

Inflammation is a highly regulated physiological response that has evolved as a mechanism to respond to infection as well as to promote healing in settings such as tissue injury. CD4+ regulatory T cells (Treg) are observed at sites of acute and chronic inflammation (Lühn, K. et al., J. Exp. Med. 2007, 204, 979-985; Miyara, M. et al., J. Exp. Med. 2006, 203, 359-370), raising questions as to the basis and consequences of their accumulation at these sites. Initial evidence suggested that immunosuppressive activities of Treg are diminished in the presence of inflammatory signals (Pasare, C., & Medzhitov, R., Science 2003, 299, 1033-1036; O'Sullivan, B. J. et al., J. Immunol. 2006, 176, 7278-7287; Oberg, H. H., et al. Immunol. 2006, 64, 353-360; Stoop, J. N. et al., Hepatology 2007, 46, 699-705). However, in a number of studies, the apparently opposite picture emerged, wherein Treg exposed to inflammatory signals retain potent suppressive activity. For instance, murine Treg at sites of viral infection or isolated from inflamed tissues still mediate regulatory function (Lund, J. M., Hsing, L., Pham, T. T. & Rudensky, A. Y., Science 2008, 320, 1220-1224; O'Connor, R. A., Malpass, K. H. & Anderton, S. M., J. Immunol. 2007, 179, 958-966; Tonkin, D. R. & Haskins, K., Eur. J. Immunol. 2009, 39, 1313-1322), as do human Treg isolated from rheumatoid joints or inflamed colonic mucosa (van Amelsfort, J. M., Jacobs, K. M., Bijlsma, J. W., Lafeber, F. P. & Taams, L. S., Arthritis Rheum. 2004, 50, 2775-2785; Holmen, N. et al., Inflamm. Bowel Dis. 2006, 12, 447-456). The present invention addresses these issues.

### SUMMARY OF THE INVENTION

Methods and compositions are provided for inducing the proliferation of regulatory T cells. These methods find a number of uses, including, for example, treating autoimmune and rheumatoid diseases. Also provided are reagents and kits that find use in these methods.

In some aspects of the invention, a method is provided for inducing the proliferation of regulatory T cells to produce an expanded population of regulatory T cells. In some embodiments, a leukocyte population comprising regulatory T cells and antigen presenting cells is contacted with a serum amyloid A (SAA) composition in an amount that is effective to induce regulatory T cell proliferation. In some embodiments, the SAA composition is a SAA polypeptide, a SAA polypeptide fragment, a polynucleotide encoding a SAA polypeptide, or a polynucleotide encoding a SAA polypeptide fragment. In some embodiments, the antigen presenting cells are monocytes. In some embodiments, the regulatory T cells are CD4⁺CD25⁺ regulatory T cells. In some embodiments, the leukocyte population is contacted in vitro. In some embodiments, the leukocyte population is contacted in vivo. In some embodiments, the method further comprises the step of measuring the proliferation of the regulatory T cells.

In some embodiments, a leukocyte population comprising regulatory T cells is contacted with a serum amyloid A (SAA) composition and interleukin 1 (IL-1); or a serum amyloid A (SAA) composition and interleukin 6 (IL-6); or a serum amyloid A (SAA) composition and both IL-1 and IL-6 in an amount that is effective to induce regulatory T cell proliferation. In some embodiments, the SAA composition is a SAA polypeptide, a SAA polypeptide fragment, a polynucleotide encoding a SAA polypeptide, or a polynucleotide encoding a SAA polypeptide fragment. In some embodiments, the regulatory T cells are CD4⁺CD25⁺ regulatory T cells. In some embodiments, the leukocyte population is contacted in vitro. In some embodiments, the leukocyte population is contacted in vivo. In some embodiments, the method further comprises the step of measuring the proliferation of the regulatory T cells.

In some aspects of the invention, a method is provided for treating autoimmune or rheumatoid disease, the method comprising administering to a subject with an autoimmune disease a SAA composition in an amount that is effective in treating the autoimmune disease. In some embodiments, the SAA composition is selected from a SAA polypeptide or fragment thereof and a polynucleotide encoding a SAA polypeptide or fragment thereof. In some embodiment, the method further comprises administering to the subject an effective amount of interleukin 1 (IL-1) and/or interleukin 6 (IL-6). In some embodiments, the method further comprises the step of measuring the proliferation of the regulatory T cells. In some embodiments, the method further comprises the step of measuring the treatment of the autoimmune disease in the subject. In some embodiments, the autoimmune or rheumatoid disease is rheumatoid arthritis, juvenile idiopathic arthritis, systemic lupus erythematosus, spondyloarthropathy, psoriatic arthritis, Kawasaki disease, Sjogren's syndrome, sarcoidosis, multiple sclerosis, type 1 diabetes mellitus, graft versus host disease, transplant rejection, ulcerative colitis or Crohn's disease.

In some aspects of the invention, a method is provided for treating autoimmune or rheumatoid disease, the method comprising administering to a subject with an autoimmune disease an effective amount of a regulatory T cell composition expanded by the methods herein to treat the autoimmune disease. In some embodiments, the regulatory T cells are CD4⁺CD25⁺ regulatory T cells. In some embodiments, the regulatory T cells are selected for prior to administration in to the subject. In some embodiments, the method further comprises the step of measuring the treatment of the autoimmune disease in the subject. In some embodiments, the autoimmune or rheumatoid disease is rheumatoid arthritis, juvenile idiopathic arthritis, systemic lupus erythematosus, spondyloarthropathy, psoriatic arthritis, Kawasaki disease, Sjogren's syndrome, sarcoidosis, multiple sclerosis, type 1 diabetes mellitus, graft versus host disease, transplant rejection, ulcerative colitis, or Crohn's disease.

### DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. SJIA plasma selectively induces human Treg proliferation in suppression assays. A. Representative data from thymidine-based suppression assays with APC. Treg and Teff were cultured separately or together in the presence of SJIA plasma, HC plasma, or complete media (FBS). Data represent average values from triplicates, comparable results were obtained using median values. B. Effects of SJIA plasma at different disease stages (flare-F n=16, quiescence-Q n=7, remission-R n=10) and HC plasma (n=30) on cell proliferation in thymidine-based suppression assays with APC. C-D. Percentages of proliferating Teff and Treg in CFSE-based suppression assays with HC plasma (n=10) and SJIA plasma (n=10) or in stimulation assays with APC. E. Representative FACS plots of CFSE-based suppression assays with APC to track proliferation of Treg and Teff in the presence of HC plasma and SJIA plasma. ANOVA (B) and unpaired two-tailed t-tests (C, D) were used for statistical analyses. Horizontal bars represented median values; bar graphs represented means and standard errors where indicated throughout the figure.

FIG. 2. Endogenous SAA is necessary for the induction of human Treg proliferation by SJIA plasma. A. SAA levels in SJIA plasma at different disease stages (flare-F n=16, quiescence-Q n=17, remission-R n=7) and HC plasma (n=14). B-C. Effects of SAA-depleted SJIA plasma on Teff and Treg proliferation in CFSE based suppression assays with APC (n=2). D. Representative FACS plots of Teff and Treg proliferation in CFSE-based suppression assays with SAA-depleted SJIA plasma in the presence of APC. Negative control for SAA depletion experiments was performed with L243 antibody (anti-HLA-DR). E-F. Effects of recombinant SAA at different doses on Treg and Teff proliferation in CFSE-based suppression assays with APC (n=2). Polymixin (5 μg/ml) was used in these cultures to block possible effects of endotoxin. G. Representative FACS plots of Treg and Teff proliferation in CFSE-based suppression assays with recombinant SAA in the presence of APC. ANOVA was used for statistical analyses. Horizontal bars represented median values; bar graphs represented means and standard errors where indicated throughout the figure.

FIG. 3. Exogenous SAA selectively induces murine Treg proliferation in vivo. A-C. Frequency of Treg out of total peritoneal CD4+ T cells and percentages of proliferating Teff and Treg in peritoneal fluid collected 16 hours after intraperitoneal injection with endotoxin (n=7), recombinant human SAA (rhSAA, n=10), and human serum albumin (HSA, n=1). Recombinant SAA and HSA were injected at 30 μg per injection in 100 μl PBS. LPS were injected at concentration similar to the level detected in recombinant SAA solution (0.25 pg/ml). D. Representative FACS plots of Treg frequency (top) and percentages of proliferating Teff and Treg (bottom) in peritoneal fluid collected 16 hours after intraperitoneal injection of different substances. Unpaired two-tailed t-tests were used for statistical analyses. Horizontal bars represented median values where indicated throughout the figure.

FIG. 4. The essential role of monocytes in the induction of Treg proliferation by SAA. A-B. Percentages of proliferating human Teff and Treg in CFSE-based suppression assays with HC plasma (n=2), SJIA plasma (n=2), recombinant SAA (25 μg/ml, n=2) or in stimulation assays (n=2) in the absence of APC. C-D. Suppression assays with SJIA plasma and HC plasma in the presence or absence of monocytes (n=5) or B cells (n=5). E-F. Frequency of Treg and percentage of proliferating Treg in peritoneal fluid collected 16 hours after intraperitoneal injection with recombinant human SAA (rhSAA) after systemic depletion of monocytes by clodronate liposomes (n=8). 400 μl of liposome solution was injected intraperitoneally 24 hours before SAA injection. G. Representative FACS plots of circulating monocyte frequency after empty vs. clodronate liposome treatment (left), Treg frequency (middle), and percentages of proliferating Teff and Treg (right) in peritoneal fluid collected 16 hours after intraperitoneal injection of different substances. Paired two-tailed t-tests (C, D) and unpaired two-tailed t-tests (E, F) were used for statistical analyses. Horizontal bars represented median values; bar graphs represented means and standard errors where indicated throughout the figure.

FIG. 5. SAA elicits robust IL-1 and IL-6 production by monocytes. A. Effects of HC plasma and SJIA plasma on Treg proliferation in CFSE-based suppression assays in the presence of fixed vs. live APC (n=5). B-C. Expression of IL-1 and IL-6 in HC and SJIA plasma samples used in suppression assays and in supernatants from suppression assays with HC plasma (plasma IL-1 n=15, plasma IL-6 n=19, supernatant IL-1/IL-6 n=18) and SJIA plasma (plasma IL-1 n=16, plasma IL-6 n=35, supernatant IL-1/IL-6 n=16) in the presence of live APC after 24 hours in culture. D. Production of IL-1 and IL-6 from APC stimulated with HC plasma (n=10) and SJIA plasma (n=8). E. Expression of IL-1 and IL-6 in monocytes in suppression assays with HC plasma and SJIA plasma after 24 hours in culture. F. IL-1 and IL-6 production from B cells and monocytes from HC PBMC (n=6) that were stimulated with 25 μg/ml of recombinant SAA in the presence of polymixin (5 μg/ml) after 24 hours in culture. G. Representative FACS plots of IL-1 and IL-6 expression in B cells and monocytes stimulated with recombinant SAA. H. Expression of TLR2, TLR4, FPRL-1, CD36, and RAGE by B cells and monocytes (n=6). I. Representative FACS plots of the expression of SAA receptors by B cells and monocytes. Unpaired two-tailed (B-E) and paired two-tailed (F-H) t-tests were used for statistical analyses. Horizontal bars represented median values where indicated throughout the figure.

FIG. 6. IL-1 and IL-6 were necessary for the induction of Treg proliferation by SAA. A. Representative data from APC-and-thymidine-based suppression assays with SJIA plasma in the presence of different concentrations of blocking IL-1 and IL-6 reagents. B. Effects of IL1-Ra, blocking antibodies against IL-6 and TNF-α on cell proliferation in thymidine-based suppression assays with APC (n=6). C-D. Percentages of proliferating Teff and Treg in APC-and-CFSE-based suppression assays with SJIA plasma in the presence of blocking IL-1 and IL-6 reagents (n=5). IL-1 Ra was used at 1 μM and blocking antibodies for IL-6 and TNF-α as well as control antibodies were used at 1 μg/ml in these experiments. E. Representative FACS plots of CFSE-based suppression assays with APC to track proliferation of Teff and Treg in the presence of SJIA plasma and blocking reagents. ANOVA (B, D) was used for statistical analyses. Horizontal bars represented median values; bar graphs represented means and standard errors where indicated throughout the figure.

FIG. 7. Mitogenic signaling cascades in Treg and Teff in suppression assays. A. Quantitation of ERK1/2, AKT, and STAT3 phosphorylation (pERK1/2, pAKT, and pSTAT3) by FACS. Teff (left) or Treg (right) were labeled with CFSE. Data were collected at different time points (day 1, 4, and 7) during suppression assays with SJIA plasma (n=6), HC plasma (n=6) or complete media (FBS, n=4). B. Effects of blocking IL-1 and IL-6 on phosphorylation status of ERK1/2, AKT, and STAT3 by Treg in suppression assays with SJIA plasma (n=6). IL-1 RA was used at 1 μM and blocking antibody for IL-6 was used at 1 μg/ml in these experiments. C. Effects of SOCS3 modulation on phosphorylation status of ERK1/2, AKT, and STAT3 by Treg and Teff in suppression assays with SJIA plasma. Treg were rested in complete media or treated with forskolin for 24 hours before being used in these assays. Data for B and C were collected at day 4 during suppression assays with SJIA plasma (n=6), HC plasma (n=6) or complete media (FBS, n=4). Unpaired two-tailed t-tests (A, C), paired two-tailed t-tests (B) were used for statistical analyses. Horizontal bars represent median values where indicated throughout the figure.

FIG. 8. Induction of SOCS3 in Treg abrogates their selective proliferation driven by endogenous SAA in SJIA plasma. A. Expression of SOCS3 in Treg and Teff (n=7). Representative FACS plots of expression of SOCS3 in Treg and Teff. B. Expression of SOCS3 in Treg that were treated with different concentrations of forskolin at different time points (n=2 experiments with similar results). Representative FACS plots of expression of SOCS3 in forskolin-treated Treg. C. Expression of SOCS3 in Treg and Teff in APC-based suppression assays with HC plasma (n=5) and SJIA plasma (n=5) after 4 days in culture. Representative FACS plots of expression of SOCS3 in Treg and Teff in APC-based suppression assays with HC plasma and SJIA plasma. D. Expression of SOCS3 in forskolin-treated Treg and untreated Teff in APC-based suppression assays with HC plasma (n=5) and SJIA plasma (n=5) after 24 hours in culture. Representative FACS plots of expression of SOCS3 in forskolin-treated Treg and untreated Teff in APC-based suppression assays with HC plasma and SJIA plasma. E-F. Percentages of proliferating Treg and Teff in APC-based suppression assays with HC plasma (n=5) and SJIA plasma (n=5) after 24 hours in culture. G-H. Percentages of proliferating forskolin-treated Treg and untreated Teff in APC-based suppression assays with HC plasma (n=5) and SJIA plasma (n=5) after 24 hours in culture. Horizontal bars represented median values where indicated throughout the figure.

FIG. 9. Characterization of the effects of SJIA plasma on cell proliferation in suppression assays. A. Titration of plasma volume used in thymidine-based suppression assays with APC (n=2, shown as triangles and squares). Plasma volume was represented as percentage of the total volume of each assay. B. Effects of washing out SJIA plasma at different days (D1-6) on cell proliferation in thymidinebased suppression assays with APC (n=5). Paired two-tailed t-tests were used for statistical analyses. C. Effects of HC plasma (n=7) and SJIA plasma (n=20) on cell proliferation of each T cell type alone (Treg or Teff) in thymidine-based stimulation assays with APC. D. Effects of dialysis of SJIA plasma with 5 kD membrane on cell proliferation in thymidine-based suppression assays with APC (n=6). E. Effects of heat inactivation of SJIA plasma on cell proliferation in thymidine-based suppression assays with APC (n=6). F. Effects of albumin depletion of SJIA plasma on cell proliferation in thymidine-based suppression assays with APC (n=5). G. Effects of different FPLC fractions of SJIA plasma on cell proliferation in thymidine-based suppression assays with APC. Pooled samples of 4 consecutive size fractions were used in suppression assays (n=2 experiments with similar results). H. SAA levels in SJIA plasma before and after depletion (n=2 experiments with similar results). Anti HLA-DR (L243) antibodies were used as negative control for the depletion experiment. Paired two-tailed t-tests (E, F) were used for statistical analyses. Horizontal bars represented median values; bar graphs represented means and standard errors where indicated throughout the figure.

FIG. 10. Expression of cytokines in plasma of HC and SJIA subjects. Expression of IFN-g, TNF-a, IL-10, IL-18, IL-4, IL-2, IL-7, and IL-15 in plasma from SJIA (Flare-F, Quiescence-Q, Remission-R) and HC subjects used in suppression assays. ANOVA was used for statistical analyses. Horizontal bars represented median values where indicated throughout the figure.

FIG. 11. Expression of cytokines in supernatants from suppression assays. Expression of IFN-g, TNF-a, IL-10, IL-18, IL-4, IL-2, IL-7, and IL-15 in supernatant from suppression assays in the presence of APC with SJIA (Flare-F, Quiescence-Q, Remission-R) and HC plasma after 24 hours. Horizontal bars represented median values where indicated throughout the figure.

FIG. 12. SJIA plasma induces an immature dendritic cell phenotype in APC in suppression assays. A. Representative FACS plots of the maturation of monocytes into DC in suppression assays with SJIA plasma (n=6 experiments with similar results). (Top) Gating of Lin−DR+ DC and Lin+DR+ cells (monocytes and B cells). (Bottom) Gated DC contained two subsets with different expression of CFSE. CFSE+ DC are derived from monocytes, and CFSE-subset represents blood DC. B. Expression of CD40, CD83, CD86, CCR7, PD-L1, PD-L2, HVEM, and DR on Lin− DR+ DC in APC-based suppression assays with SJIA plasma and HC plasma at day 4 in culture (n=6); and IL-4/GM-CSF-induced monocyte-derived immature and mature DC. C. Representative FACS plots of CD40, CD83, CD86, CCR7, PD-L1, PD-L2, HVEM, and DR expression by DC in suppression assays with SJIA plasma and HC plasma; and IL-4/GM-CSF-induced monocyte-derived immature and mature DC. Unpaired two-tailed t-tests were used for statistical analyses. Horizontal bars represented median values where indicated throughout the figure.

FIG. 13. Mitogenic signaling cascades in Treg and Teff in suppression assays. A. Representative FACS plots of phosphorylated ERK1/2, AKT, and STAT3 (pERK1/2, pAKT, and pSTAT3) in Treg and Teff in suppression assays with SJIA and HC plasma. B. Representative FACS plots of phosphorylated ERK1/2, AKT, and STAT3 in Treg in suppression assays in the presence of blocking reagents of IL-1 and IL-6 signaling. C. Representative FACS plots of phosphorylated ERK1/2, AKT, and STAT3 in Treg and Teff in suppression assays with SJIA and HC plasma in the presence or absence of forskolin-treated Treg.

FIG. 14. SOCS3 regulates mitogenic signaling cascades in Treg and Teff in suppression assays. A. Intra-assay comparison of ERK1/2, AKT, and STAT3 phosphorylation (pERK1/2, pAKT, and pSTAT3) between Treg and Teff in suppression assays with SJIA plasma. Data were collected at day 1, 4, and 7 (n=6). B. Intra-assay comparison of ERK1/2, AKT, and STAT3 phosphorylation between Treg and Teff in cultures with forskolin-treated Treg and untreated Treg. Data were collected at day 4 (n=5). C. Inter-assay comparison of ERK1/2, AKT, and STAT3 phosphorylation in Treg and Teff between cultures with forskolin-treated Treg and untreated Treg. Data were collected at day 4 (n=5). Paired two-tailed t-tests (A, B) and unpaired two-tailed t tests (C) were used for statistical analyses. Horizontal bars represent median values where indicated throughout the figure.

FIG. 15. Surface phenotype of Treg and Teff in suppression assays. A-B. Expression of components of IL-1 and IL-6 receptor complexes (IL-1R1, gp130) in Teff and Treg in suppression assays. Data were collected at different time points (day 1, 4, and 7) during suppression assays with SJIA plasma (n=6), HC plasma (n=6). Unpaired two-tailed t-tests were used for statistical analyses. Horizontal bars represented median values where indicated throughout the figure.

FIG. 16. Human Treg isolation. (Left) Representative FACS plot of the gating strategy for identification of circulating Treg from purified CD4+ T cells. Co-staining with CD127 and CD25 identified the Treg and Teff subsets. Gates for CD4+CD25+CD127Io/− Treg and CD4+CD25− Teff were established based on negative thresholds from staining with isotype control antibodies. (Right) Purity of sorted Treg and Teff were examined by co-staining for CD25 and Foxp3.

### DETAILED DESCRIPTION OF THE EMBODIMENTS

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Methods and compositions are provided for inducing the proliferation of regulatory T cells. These methods find a number of uses, including, for example, treating autoimmune and rheumatoid diseases. Also provided are reagents and kits that find use in these methods. These methods are based on the observations that endogenous plasma-derived serum amyloid A (SAA) induces proliferation of regulatory T cells (Treg) while maintaining their suppressive activities. Administration of exogenous SAA in mice selectively enhances local abundance of proliferating Treg in a monocyte-dependent manner. SAA elicits robust production of IL-1 and IL-6 from monocytes and its effects on Treg expansion are abrogated by neutralizing these cytokines. The two cytokines activate distinct signaling pathways in Treg. The selective response of Treg to SAA correlates with their diminished expression of SOCS3 and is antagonized by Treg-specific induction of SOCS3. These results indicate that SAA induces a cytokine and cellular milieu that supports Treg expansion at sites of infection or tissue injury, which in turn curbs inflammatory and autoimmune responses.

### Methods and Compositions

In aspects of the invention, methods are provided for inducing the proliferation of regulatory T cells. Regulatory T cells (“Treg”) are a specialized subpopulation of T cells which suppresses activation of the immune system and thereby maintains tolerance to self-antigens. There are various types of regulatory T cells. The majority of recent research has focused on TCRαβ+CD4+ regulatory T cells. These include natural regulatory T cells (nTreg), which are T cells produced in the thymus and delivered to the periphery as a long-lived lineage of self-antigen-specific lymphocytes; and induced regulatory T cells (iTreg), which are recruited from circulating lymphocytes and acquire regulatory properties under particular conditions of stimulation in the periphery. Both cell types are CD4+CD25+, both can inhibit proliferation of CD4+CD25− T cells in a dose dependent manner, and both are anergic and do not proliferate upon TCR stimulation. In addition to being positive for CD4 and CD25, regulatory T cells are positive for the transcription factor Foxp3, an intracellular marker.

A key characteristic of regulatory T cells is their anergy. In contrast to CD4+CD25− T cells, which proliferate upon receiving T cell receptor (TCR) stimulation, regulatory T cells are unresponsive to this proliferative signal on its own. However, regulatory T cells cultured with anti-CD3 antibodies (for TCR stimulation) and excess exogenous IL-2 (a T cell growth factor) overcome anergy and proliferate; blocking IL-2 inhibits this phenomenon. The anergic state of regulatory T cells can also be overcome by anti-CD28 costimulation or interaction with mature dendritic cells.

A second cardinal feature of regulatory T cells is their ability to suppress immune responses. Suppression occurs when regulatory T cells are activated with antigens recognized by their specific TCR, but can be maintained without further TCR stimulation. Thus, suppressive activity is antigen-nonspecific. However, regulatory T cells that share the same antigenic specificity with effector cells are more suppressive. Similarly, allogeneic regulatory T cells are suppressive, but autologous regulatory T cells are more potent suppressors. The main targets of suppression by regulatory T cells are innate and adaptive immune cells. These regulatory T cells also participate in immune responses against infectious agents, malignant cells, and allogeneic organ and stem cell grafts.

As discussed above, regulatory T cells are identifiable from other leukocytes in that they express the cell surface markers CD4 and CD25 and the intracellular marker FoxP3. That is to say, they are positive for CD4, CD25, and FoxP3. It will be understood by those of skill in the art that the stated expression levels reflect detectable amounts of the marker protein on the cell surface. A cell that is negative for staining (the level of binding of a marker specific reagent is not detectably different from an isotype matched control) may still express minor amounts of the marker. And while it is commonplace in the art to refer to cells as “positive” or “negative” for a particular marker, actual expression levels are a quantitative trait. The number of molecules on the cell surface can vary by several logs, yet still be characterized as “positive”.

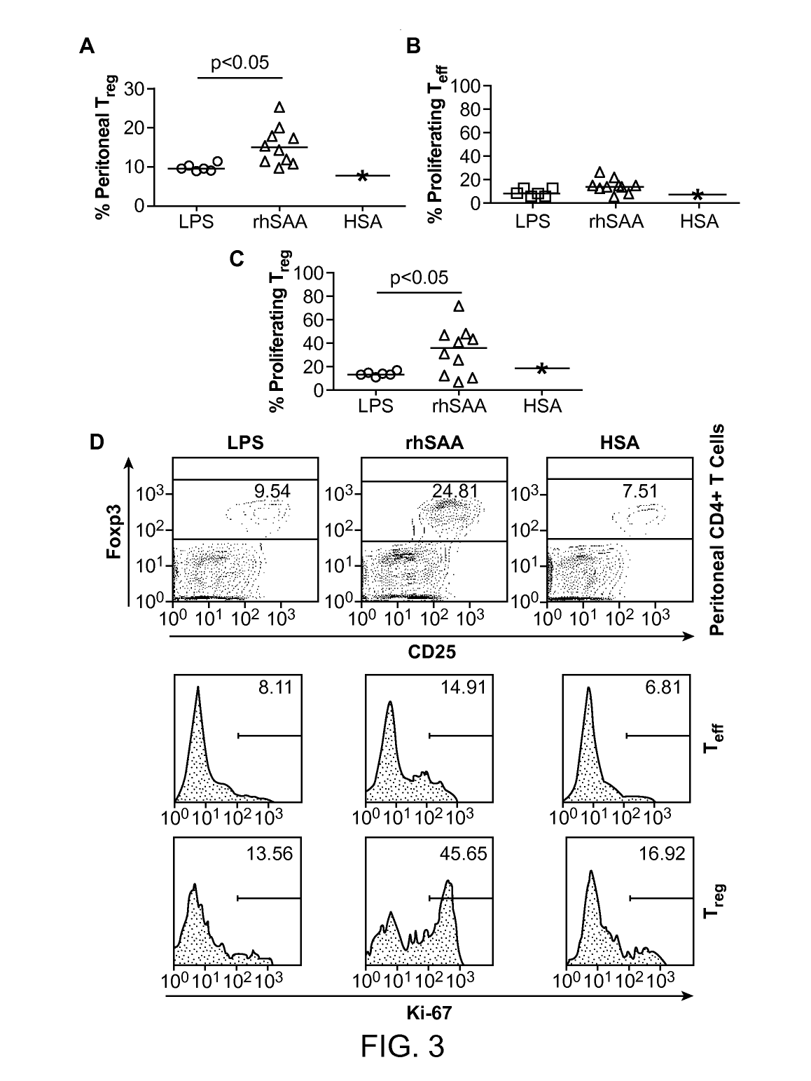

In some embodiments, methods of inducing the proliferation of regulatory T cells are employed to produce an enriched population of regulatory T cells. By an “enriched population of regulatory T cells”, it is meant that the representation of regulatory T cells in the cell population is greater than would otherwise be, e.g., in the absence of the methods provided. For example, a complete leukocyte sample harvested from the peritoneum of a mouse challenged with endotoxin is a heterogeneous population of cells that comprises only about 9% regulatory T cells. However, following methods of the invention, a heterogenous population of cells is produced in which about 25% of the cells or more are regulatory T cells (see, e.g., FIG. 3D). In other words, methods of the invention increase the percentage of regulatory T cells in the population by at least 1.5 fold or more, e.g. 2-fold or more, in some instances 3-fold or more, relative to the number of regulatory T cells that would exist in the cell population in the absence of performing the methods provided herein.

In some embodiments, methods of inducing the proliferation of regulatory T cells are employed in the treatment of autoimmune or rheumatic diseases, for example, by providing an enriched population of regulatory T cells produced ex vivo to a subject, or by inducing the proliferation of regulatory T cells in vivo in a subject. By “autoimmune disease” it is meant a disease that arises from an overactive immune response of the body against substances and tissues normally present in the body. In other words, the body actually attacks its own cells. By “rheumatic disease” it is meant a chronic inflammatory condition affecting the loco-motor system including joints, muscles, connective tissues, soft tissues around the joints and bones, e.g. rheumatoid arthritis, ankylosing spondylitis, gout and systemic lupus erythematosus.

Regulatory T cells have a role in autoimmune and rheumatic diseases. Human patients with Foxp3 gene mutations develop IPEX syndrome, a disease characterized by immune dysregulation. In addition to IPEX, many more common polygenic autoimmune disorders, including multiple sclerosis, and type 1 diabetes, are distinguished by irregularities in regulatory T cell distribution and function (Milojevic D et al., Pediatric Rheumatology 2008, 6, 20); and colitis has been found to have a negative impact on regulatory T cell development in the thymus in a mouse model of this disease, having profound implications for the treatment of human inflammatory bowel disease (Faubion W A et al., Gastroenterology 2004, 126, 1759-70). Rheumatic diseases, including juvenile idiopathic arthritis, rheumatoid arthritis, systemic lupus erythematosus, spondyloarthropathy, Kawasaki disease, Sjögren's syndrome, and sarcoidosis, are characterized by abnormalities in CD4+CD25+ regulatory T cell distribution and function (Milojevic D et al., Pediatric Rheumatology 2008, 6, 20, Table 2).

Table 1 below provides the basis for the treatment of disease with regulatory T cells. The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease. The terms “individual,” “subject,” “host,” and “patient,” are used interchangeably herein and refer to any mammalian subject for whom diagnosis, treatment, or therapy is desired, particularly humans.

TABLE 1

| | Freq. | Freq. | Func- | |
|---|---|---|---|---|
| Autoimmune | in Blood | at Site | tion | |
| Disease | a | a, b | c | Reference |
| ALPS d | ↓ | | — | Blood |
| | | | | 2001; 98: 2466-2473 |
| APSI/APECED e | ↓ | | — | J Allergy Clin |
| | | | | Immunol |
| | | | | 2005; 116: 1158- |
| | | | | 1159* |
| APSII f | [symbol] | | ↓ | J Exp Med |
| | | | | 2004; 199: 1285- |
| | | | | 1291* |
| Arthritis g: | ↓ | ↑ | ↑ h | J Immunol |
| Juvenile | | | | 2004; 172: 6435- |
| Idiopathic | | | | 6443* |
| Arthritis | ↓ | ↑ | [symbol] | Arthritis Res Ther |
| | | | | 2004; 6: R335-R346 |
| Arthritis g: | ↓ | ↑ | [symbol] | Arthritis Res Ther |
| Psoriatic | | | | 2004; 6: R335-R346 |
| Arthritis | | | | |
| Arthritis g: | ↑ | ↑ | [symbol] ↑ i, j | Arthritis Rheum |
| Rheumatoid | | | | 2004; 50: 2775-2785 |
| Arthritis | [symbol] | ↑ | [symbol] ↑ i | Clin Exp Immunol |
| | | | | 2005; 140: 360-367* |
| | — | | ↓ k | J Exp Med |
| | | | | 2004; 200: 277-285 |
| | ↓ | ↑ | [symbol] | Arthritis Res Ther |
| | | | | 2004; 6: R335-R346 |
| Arthritis g: | ↓ | ↑ | [symbol] | Rheumatology |
| Early Active RA | | | | (Oxford) |
| | | | | 2006; 45: 1210- |
| | | | | 1217* |
| Arthritis g: | ↓ | ↑ | [symbol] | Arthritis Res Ther |
| Spondyloar- | | | | 2004; 6: R335-R346 |
| thropathy | | | | |
| Atopic | [symbol] | | [symbol] | Br J Dermatol |
| Dermatitis 1 | | | | 2005; 153: 750-757 |
| | [symbol] | | [symbol] | J Allergy Clin |
| | | | | Immunol |
| | | | | 2006; 117: 176-183 |
| Autoimmune | ↓ | | ↓ | J Immunol |
| Hepatitis | | | | 2006; 176: 4484- |
| | | | | 4491* |
| | ↓ | | [symbol] | Hepatology |
| | | | | 2006; 43: 729-737* |
| Autoimmune | [symbol] | | [symbol] | J Clin Endocrinol |
| Thyroiditis | | | | Metab |
| | | | | 2006; 91: 3639- |
| | | | | 3646* |
| Celiac Disease | ↑ | | — | Clin Exp Immunol |
| | | | | 2006; 144: 67-75 |
| Crohn's Disease | | | [symbol] m | Clin Exp Immunol |
| | | | | 2005; 141: 549-557 |
| Diabetes | ↓ | | | J Clin Invest |
| Mellitus | | | | 2002; 109: 131-140 |
| Type I | [symbol] | | ↓ | Diabetes |
| | | | | 2007; 56: 604-612* |
| | [symbol] | | [symbol] | J Autoimmun |
| | | | | 2005; 24: 55-62 |
| | [symbol] | | [symbol] n | J Immunol |
| | | | | 2006; 177: 8338- |
| | | | | 8347* |
| | [symbol] | | ↓ o | Diabetes |
| | | | | 2005; 54: 92-99 |
| | [symbol] | | [symbol] p | J Exp Med |
| | | | | 2006; 203: 1701- |
| | | | | 1711* |
| HCV Mixed | [symbol] ↓ | | [symbol] | Blood |
| Cryoglobulin- | | | | 2004; 103: 3428- |
| aemia q | | | | 3430* |
| ITP r | ↓ | | ↓ | Zhonghua Xue Ye |
| | | | | Xue Za Zhi |
| | | | | 2007; 28: 184-188* |
| IPEX s | ↓ | | ↓ | J Clin Invest |
| | | | | 2006; 116: 1713- |
| | | | | 1722* |
| IBD t | [symbol] | ↑ | [symbol] | J Immunol |
| | | | | 2004; 173: 3119- |
| | | | | 3130* |
| Kawasaki's | ↓ | | — | J Pediatr |
| Disease u | | | | 2004; 145: 385-390* |
| Multiple | [symbol] | | — | J Clin Immunol |
| Sclerosis | | | | 2004; 24: 155-161 |
| | [symbol] | | [symbol] ↓ v | J Neurosci Res |
| | | | | 2006; 83: 1432- |
| | | | | 1446* |
| | ↓ w | | ↓ | J Neurosci Res |
| | | | | 2005; 81: 45-52* |
| | [symbol] | | ↓ | J Exp Med |
| | | | | 2004; 199: 971-979 |
| | — | | ↓ | J Immunol Methods |
| | | | | 2007; 322: 1-11* |
| Myasthenia | [symbol] | | ↓ x | Blood |
| Gravis | | | | 2005; 105: 735-741* |
| | [symbol] | | — | J Biol Regul |
| | | | | Homeost Agents |
| | | | | 205; 19: 54-62 |
| | ↓ | | — | Immunology |
| | | | | 2005; 116: 134-141 |
| Sjögren's | ↑ | | [symbol] | J Autoimmun |
| Syndrome | | | | 2005; 24: 235-242 |
| Sarcoidosis | [symbol] | | [symbol] | J Exp Med |
| | | | | 2006; 203: 359-370* |
| | ↑ | ↑ | ↓ | J Exp Med |
| | | | | 2006; 203: 359-370* |
| SLE y | ↓ | | — | J Autoimmun |
| | | | | 2003; 21: 273-276 |
| | [symbol] | | ↓ z | J Autoimmun |
| | | | | 2006; 27: 110-118 |
| | ↓ | | ↓ | Adv Exp Med Biol |
| | | | | 2007; 601: 113-119* |
| | ↓ | | — | Scand J Immunol |
| | | | | 2004; 59: 198-202 |
| Pediatric SLE | ↓ aa | | — | Immunology |
| | | | | 2006; 117: 280-286* |
| Ulcerative | [symbol] | | [symbol] | Inflamm Bowel Dis |
| Colitis | | | | 2007; 13: 191-199* |
| | ↓ bb | | — | Dig Dis Sci |
| | | | | 2006; 51: 677-686* |
| | ↓ cc | | — | World J Surg |
| | | | | 2006; 30: 590-597 |
| Vasculitis | ↑ dd | | — | Clin Exp Immunol |
| | | | | 2005; 140: 181-191* |

↑ increased compared to normal controls (NC);
↓ decreased compared to NC; [symbol]
no difference compared to NC;
— not done/evaluated;
*Foxp3 was evaluated in these publications;
a, Frequency—percentage of CD4+CD25+ cells unless otherwise indicated;
b, Site—site of inflammation (joints/synovial fluid in arthritis, mucosa in IBD, bronchoalveolar (BAL) fluid and lymph nodes in sarcoidosis);
c, Function—suppression of proliferation or inhibition of cytokine production;
d, ALPS—Autoimmune Lymphoproliferative Syndrome;
e, APSI/APECED—Autoimmune Polyglandular Syndrome type 1/autoimmune polyendocrinopathy candidiasis ectodermal dystrophy;
f, APSII—Autoimmune Polyglandular Syndrome type 2;
g, Function is relatively stable over time and independent of disease activation/inflammation;
h, i Treg isolated from synovial fluid of juvenile idiopathic arthritis (JIA) and rheumatoid arthritis (RA) patients suppress better than those isolated from peripheral blood;
j, k, Treg are CD4+CD25+;
l, Treg are CD4+CD25+;
m, Treg isolated from gut;
n, Function normal in expanded cells - freshly isolated cells not tested;
o, Treg are CD4+CD25+;
p, Treg are CD4+CD25+CD127lo/-;
q, Frequency is normal in asymptomatic patients and decreased in symptomatic individuals;
r, ITP—idiopathic thrombocytopenic purpura - CD4+CD25+ increased, CD4+Foxp3+ and CD4+CD25+Foxp3+ decreased;
s, IPEX—immune dysregulation, polyendocrinopathy, enteropathy, Xlinked - frequency varies depending on expression levels of functional Foxp3 protein;
t, IBD—inflammatory bowel disease;
u, Low Foxp3, GITR and CTLA-4 expression;
v, multiple sclerosis—MS, normal in SPMS (slow progressing MS)/decreased in relapsing/remitting MS (RRM);
w, Decreased Foxp3 expression;
x, Increased HLADR and Fas and decreased Foxp3 expression;
y, SLE—systematic lupus erythematosus;
z, Treg are CD4+CD25+;
aa, Treg are CD4+CD25+;
bb, Decreased in active disease;
cc, Treg are CD4+CD25+CD45RA+;
dd, ANCA—Antineutrophil cytoplasmic antibodies, Treg are CD4+CD25+.

Rheumatoid Arthritis.

Rheumatoid arthritis (RA) is a rheumatic and autoimmune disease characterized by inflammation in the joints. The 1987 American College of Rheumatology criteria are used in the clinical diagnosis of rheumatoid arthritis, and to define rheumatoid arthritis in epidemiologic studies. Persons must meet four of seven ACR criteria; these criteria are based on clinical observation (e.g., number of joints affected), laboratory tests (e.g., positive rheumatoid factor), and radiographic examination (e.g., X-rays evidence of joint erosion) (Arnett F C et al., Arthritis Rheum 1988, 31, 315-324). Historically, pharmacologic treatment of RA has traditionally followed the pyramid approach. That is, treatment starts with corticosteroids/non-steroidal anti-inflammatory drugs, then progresses to disease-modifying anti-rheumatic drugs and finally to biologic response modifiers if persons are non-responsive to the previous drugs. (Arthritis Rheum 2002, 46, 328-346). Juvenile Idiopathic Arthritis Juvenile idiopathic arthritis (JIA) is persistent or recurring inflammation of the joints similar to rheumatoid arthritis but beginning at or before age 16. The International League Against Rheumatism (ILAR) Criteria 1997 for JIA are published as Petty R E et al., J Rheumatol 1998, 25, 1991-4. The ILAR Criteria 2001 Update is published as Petty R E et al., J Rheumatol 2004, 31, 390-2.

Systemic LUPUS Erythematosus.

Systemic lupus erythematosus (SLE) is a rheumatic and autoimmune disease characterized by inflammation that can involve joints, skin, kidneys, mucous membranes, and blood vessel walls. The American College of Rheumatology (ACR) 1982 Revised Criteria for SLE are published as Tan E M et al., Arthritis Rheum 1982, 25, 1271-7. The ACR 1982 Revised Criteria for SLE Update is published as Hochberg MC. Arthritis Rheum 1997, 40, 1725.

Spondyloarthropathy.

Spondyloarthropathy is any joint disease of the vertebral column. Spondyloarthropathy with inflammation is called spondylarthritis In the broadest sense, the term spondyloarthropathy includes joint involvement of vertebral column from any type of joint disease, including rheumatoid arthritis and osteoarthritis. The European Spondylarthropathy Study Group (ESSG) Criteria for SpA are published as Dougados M et al., Arthritis Rheum 1991, 34, 1218-27. The Amor Criteria for SpA are published as Amor B, Dougados M, Mijiyawa M. Rev Rhum Mal Osteoartic 1990, 57, 85-9.

Psoriatic Arthritis.

Psoriatic arthritis (PsA) is a chronic inflammatory arthritis that occurs mostly in people with psoriasis of the skin or nails. The original diagnostic criteria are published as Moll J M, Wright V, Semin Arthritis Rheum 1973, 3, 55-78. Minor modifications have been made to the Moll and Wright criteria by a number of authors including Veale D, Rogers S, FitzGerald O. Br J Rheumatol 1994, 33, 133-8. The Classification of Psoriatic Arthritis (CASPAR) study group is an international group of investigators that published criteria for PsA as Taylor W et al., Arthritis Rheum 2006, 54, 2665-73.

Kawasaki Disease.

Kawasaki disease (KD) is a rheumatic and autoimmune disease characterized by inflammation in the walls of blood vessels in the heart and throughout the body, with children being most vulnerable. The American Heart Association 2001 Diagnostic Criteria for KD are published as Circulation 2001, 103, 335-6.

Sjögren's Syndrome.

Sjögren's syndrome (SS) is a rheumatic and autoimmune disease characterized by dry eyes, mouth, vagina, or a combination; or red, painful eyes caused by inflammation. The American College of Rheumatology (ACR) Criteria for SS are published as Fox R1 et al., Arthritis Rheum 1986, 29, 577-85.

Sarcoidosis.

Sarcoidosis is a rheumatic and autoimmune disease in which abnormal collections of inflammatory cells (granulomas) form in many organs of the body. A comprehensive review of sarcoidosis is published as Newman, L S, Rose, C S, and Maier, L A, N Engl J Med 1997, 336, 1224-1234.

Multiple Sclerosis.

Multiple sclerosis (MS) is an autoimmune disease characterized by demyelination and subsequent axonal degeneration. The International Panel on the Diagnosis of Multiple Sclerosis Criteria 2001 for MS (“The McDonald Criteria”) are published as McDonald W I et al., Ann Neurol 2001, 50, 121-127. The revised criteria (“The Revised McDonald Criteria”) are published as Polman C H et al., Ann Neurol 2005, 58, 840-846.

Type 1 Diabetes Mellitus.

Diabetes mellitus (DM) consists of a group of syndromes characterized by hyperglycemia; altered metabolism of lipids, carbohydrates, and proteins; and an increased risk of complications from vascular disease. Most patients can be classified clinically as having either type 1 or type 2. Type 1 diabetes mellitus is an autoimmune disease; it is also called “juvenile onset” diabetes. In contrast, type 2 diabetes mellitus, which is not an autoimmune disease, is often called “adult onset” diabetes. DM or carbohydrate intolerance also is associated with certain other conditions or syndromes. Criteria for the diagnosis of DM have been proposed by several medical organizations. The American Diabetes Association (ADA) criteria include a random plasma glucose concentration of greater than 200 mg/dl, a fasting plasma glucose concentration of greater than 126 mg/dl, or a plasma glucose concentration of greater than 200 mg/dl 2 hours after the ingestion of an oral glucose load. (Diabetes Care 2003, 26, s5-20)

Graft Versus Host Disease.

Graft-versus-host disease (GVHD) is a complication that can occur after a stem cell or bone marrow transplant in which the newly transplanted material immunologically attacks the transplant recipient's body. Acute GVHD is graded based on Glucksberg H et al., Transplantation 1974, 18, 295-304, and revised by Thomas E D et al., N Engl J. Med. 1975, 292, 832-43, and Thomas E D et al., N Engl J. Med. 1975, 292, 895-902.

Transplant Resection.

Transplant rejection is when a transplant recipient's immune system attacks a transplanted organ or tissue. An international grading system has been developed for kidney (Kidney Int 1993, 44, 411-422), heart (J Heart Transplant 1990, 9, 587-593), lung (J Heart Transplant 1990, 9, 593-601), liver (Hepatology 1997, 25, 658-663), and pancreas (Am J. Transplant. 2008, 8, 1237-49), and one for skin-containing composite tissue is in progress (Am J. Transplant. 2008, 8, 1396-400).

In aspects of the invention, regulatory T cells are induced, or stimulated, to proliferate by contacting the cells with a Serum Amyloid A (SAA) composition. A SAA composition is a composition comprising SAA protein or a fragment thereof, or a nucleic acid that encodes a SAA protein or fragment thereof. Serum amyloid A (SAA) proteins are apolipoproteins that are associated with high-density lipoprotein (HDL) in plasma. The SAA1 and SAA2 human genes encode the acute phase SAAs and are clustered on human chromosome 11 (Steel D M, Whitehead A S, Immunology Today 1994, 15, 81-88; Sellar G C et al., Genomics 1994, 23, 492-495). Although allelic variation of SAA does occur at the SAA locus producing transcript variants and polymorphic proteins, the SAA1 and SAA2 genes are almost identical with respect to the primary structures of their specified products, their gene organizations and sequences, and their mode of expression.

SAA proteins circulate in the blood at a level of 1-5 μg/ml in plasma, increasing 500-1000 fold within 24 hours of an inflammatory stimulus. The human SAA gene codes for a 122 amino acid nonglycosylated protein, which contains an 18 amino acid signal peptide; thus the mature protein contains 104 amino acid residues. Peprotech sells a recombinant human SAA1 that corresponds to a human SAA1 except for the presence of an N-terminal methionine.

Sequence information for SAA proteins and the genes encoding them is available from GenBank as follows. The polypeptide sequence for human SAA1 and the nucleic acid sequence that encodes it is published as Genbank Accession Nos. NM_{—}000331.4 (variant 1) (SEQ ID NO:1 and SEQ ID NO:2), NM_{—}199161.3 (variant 2) (SEQ ID NO:3 and SEQ ID NO:4) and NM_{—}001178006.1 (variant 3) (SEQ ID NO:5 and SEQ ID NO:6). The polypeptide sequence for the human SAA2 gene and the nucleic acid sequence that encodes it is published as Genbank Accession Nos. NM_{—}030754.4 (isoform a) (SEQ ID NO:7 and SEQ ID NO:8) and NM_{—}001127380.2 (isoform b) (SEQ ID NO:9 and SEQ ID NO:10). The term SAA protein or the like refers to a polypeptide of mammalian origin, e.g., mouse or human SAA, or, as context requires, a polynucleotide encoding such a polypeptide, and has at least one of the following features: (1) an amino acid sequence of a naturally occurring mammalian SAA polypeptide, or a fragment thereof; (2) an amino acid sequence substantially identical to, e.g., 70% or more, 75% or more, 80% or more, 85% or more, 90% or more, 95% or more, 98% or more, or 99% or more, i.e. 100% identical to, an amino sequence encoding a naturally occurring mammalian SAA polypeptide, or a fragment thereof; (3) an amino acid sequence that is encoded by a naturally occurring mammalian SAA nucleotide sequence, or a fragment thereof; (4) an amino acid sequence encoded by a nucleotide sequence degenerate to a naturally occurring mammalian SAA nucleotide sequence, or a fragment thereof; or (5) an amino acid sequence encoded by a nucleotide sequence that hybridizes under low or high stringency conditions to a naturally occurring mammalian SAA nucleotide sequence, or a fragment thereof. By fragment is meant an active fragment, that is to say, a fragment that substitutes for SAA in a suppression assay, a fragment that activates the proliferation of regulatory T cells, etc. General methods for the production, purification and use of polynucleotides and polypeptides are discussed in greater detail below.

To induce the proliferation of regulatory T cells, a leukocyte population comprising regulatory T cells is contacted with an effective amount of a SAA composition. An effective amount or effective dose of a SAA composition is the amount to induce 10% or more of the Treg to proliferate. That is to say, an effective dose of a SAA composition will induce 10% or more, 20% or more, 30% or more, or 40% or more of the regulatory T cells to enter mitosis, in some instances 50% or more, 60% or more, or 70% or more of the regulatory T cells to enter mitosis, sometimes 80% or more, 90% or more, 95% or more, e.g. 100%. In other words, the proportion of regulatory T cells in the population will increase by 1.5-fold or more, e.g. 2-fold or more, 3-fold or more, 5-fold or more, or 6-fold or more, to produce an expanded population of regulatory T cells. The amount of regulatory T cell proliferation may be measured by any convenient method. For example, the number of Tregs may be determined after contact with the SAA composition, e.g. 2 hours, 4 hours, 8 hours, 12 hours, 24 hours, 36 hours, 48 hours, 72 hours or more after contact with the SAA composition, and that number compared to the number of Tregs prior to contact with the SAA composition. As another example, the number of Tregs undergoing mitosis after contacting with SAA may be measured, e.g. by measuring the number of regulatory T cells expressing proliferation markers such as nuclear antigen Ki67, and comparing that number to the number of Tregs undergoing mitosis prior to contacting with SAA. As another example, the number of new Tregs after contact with SAA may be measured, e.g. by labeling new cells with BrdU, H³-thymidine, etc. as they are being created, and comparing that number to the number of new Tregs prior to contacting with SAA.

In a clinical sense, an effective amount or effective dose of a SAA composition is the dose that, when administered for a suitable period of time, usually at least about one week, and maybe about two weeks, or more, up to a period of about 4 weeks, 8 weeks, or longer will evidence an alteration in the symptoms associated with the autoimmune or rheumatoid disease being treated. For example, an effective dose is the dose that when administered for a suitable period of time, usually at least about one week, and may be about two weeks, or more, up to a period of about 4 weeks, 8 weeks, or longer will reduce inflammation and pain by at least about 10%, e.g. 10% or more, 20% or more, 30% or more, sometimes about 40% or more, 50% or more, 60% or more, e.g. 70% or more, 80% or more, 90% or more. It will be understood by those of skill in the art that an initial dose may be administered for such periods of time, followed by maintenance doses, which, in some cases, will be at a reduced dosage.

The calculation of the effective amount or effective dose of a SAA composition to be administered is within the skill of one of ordinary skill in the art, and will be routine to those persons skilled in the art. Needless to say, the final amount to be administered will be dependent upon the route of administration and upon the nature of the disorder or condition that is to be treated. The effective amount given to a particular patient will depend on a variety of factors, several of which will differ from patient to patient. A competent clinician will be able to determine an effective amount of a therapeutic agent to administer to a patient to halt or reverse the progression the disease condition as required. Utilizing LD₅₀ animal data, and other information available for the agent, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

In some aspects of the invention, the SAA composition is employed to induce regulatory T cell proliferation ex vivo, e.g. to produce an enriched population of regulatory T cells. In such ex vivo embodiments, the regulatory T cell population to be expanded may be harvested from an individual. Regulatory T cells may be harvested by any convenient method, e.g., apheresis, leukocytapheresis, density gradient separation, etc. In apheresis, a blood sample is passed through a machine that separates out certain components, e.g. platelets, erythrocytes, plasma, or leukocytes, and returns the remaining blood components to the blood stream. In leukocytapheresis, leukocytes are selectively removed. In density gradient centrifugations, a whole blood sample may be collected and fractionated by centrifugations, and the buffy coat (comprising the leukocytes) isolated for use. Such methods yield a complete leukocyte sample, i.e. a heterogenous sample comprising both regulatory T cells and other leukocytes. In some instances, the complete leukocyte sample is then subjected to the methods described herein. In some instances, the regulatory T cells may be further isolated from other leukocytes by methods described further below for enriching for regulatory T cells, and the isolated regulatory T cells are then subjected to the methods described herein. In any case, the leukocyte sample comprising the regulatory T cells, be it a complete leukocyte sample or an enriched population of regulatory T cells, may be used immediately, or it may be stored, frozen, for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells. General methods of harvesting, culturing and storing cells are discussed in greater detail below.

To induced regulatory T cell proliferation, the SAA composition is provided to a leukocyte sample comprising regulatory T cells. In some embodiments, the leukocyte population comprises antigen presenting cells. Antigen presenting cells are leukocytes that display foreign antigen complexes with major histocompatibility complex II (MHCII) on their surfaces. Examples of antigen presenting cells of interest include monocytes (CD14+), macrophages (e.g. Mac1+), and dendritic cells (BDCA-2+, BDCA-3+, or BDCA-4+). In embodiments in which a complete leukocyte sample is employed, e.g. in in vitro embodiments that employ a complete leukocyte sample, or in in vivo embodiments, antigen presenting cells are typically present in the leukocyte sample. In embodiments in which an enriched population of regulatory T cells is employed, antigen presenting cells may be added to the leukocyte sample, i.e. to form a coculture of regulatory T cells and antigen presenting cells. In such instances, the antigen presenting cells may be from any convenient source, and may have been isolated by any convenient method, e.g. by the affinity separation techniques described below for isolating regulatory T cells, but instead using affinity agents that selectively bind to markers for antigen presenting cells, e.g. CD14 for monocytes, Mac-1 for macrophages, BDCA-2+, BDCA-3+, or BDCA-4+ for dendritic cells, etc.

Additionally, i.e. when antigen presenting cells are present, or alternatively, i.e. if antigen present cells are not present in the leukocyte sample, interleukin 1 and/or interleukin 6 may also be provided to the regulatory T cells. Interleukin-1 (IL-1α, Genbank Accession No. NM_{—}000575.3; IL-1β, Genbank Accession No. NM_{—}000575.3) and IL-6 (Genbank Accession No. NM_{—}000600.3) are cytokines that are produced by activated leukocytes and are important mediators of cellular responses during the inflammatory response. If IL-1 and/or IL-6 are provided to the regulatory T cells, antigen presenting cells are not required to be present in the leukocyte sample. On the other hand, if IL-1 and/or IL-6 are not provided to the regulatory T cells, then antigen presenting cells should be present in the leukocyte sample.

The leukocyte sample comprising regulatory T cells is contacted with the SAA composition (and, in some embodiments, IL-1 and/or IL-6) for one or more days, e.g. for two or more days, for 3 or more days, for example, for 4 or more days, for 5 or more days, for 6 or more days, for 7 or more days, in some instances for 8 or more days, for 9 or more days, for 10 or more days, for 12 or more days, for 15 or more days. Contacting may occur in any culture media and under any culture conditions that promote the survival of leukocytes. For example, the leukocytes may be conveniently suspended in an appropriate nutrient medium, such as Iscove's modified DMEM or RPMI 1640, normally supplemented with fetal calf serum (about 5-10%), L-glutamine, a thiol, particularly 2-mercaptoethanol, and antibiotics, e.g. penicillin and streptomycin. The culture may contain growth factors to which the leukocytes are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

Following these methods, regulatory T cells will be induced to proliferate ex vivo. In some embodiments, an enriched population of regulatory T cells may be produced. In some instances, the population of cells may be further enriched for regulatory T cells by separating the regulatory T cells from the heterogeneous population. Separation of the regulatory T cells from the remaining cells in the culture may be by any convenient separation technique. For example, the regulatory T cells may be separated from the heterogeneous population by affinity separation techniques. Techniques for affinity separation may include magnetic separation using magnetic beads coated with an affinity reagent, affinity chromatography, “panning” with an affinity reagent attached to a solid matrix, e.g. plate, cytotoxic agents joined to an affinity reagent or used in conjunction with an affinity reagent, e.g. complement and cytotoxins, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide). Any technique may be employed which is not unduly detrimental to the viability of the subject Tregs.

To separate the Tregs by affinity separation techniques, cells that are not Tregs may be depleted from the population by contacting the population with affinity reagents that specifically recognize and selectively bind markers that are not expressed on Tregs, e.g. B220. Additionally or alternatively, positive selection and separation may be performed using by contacting the population with affinity reagents that specifically recognize and selectively bind markers associated with Tregs, e.g. CD4 and/or CD25. By “selectively bind” is meant that the molecule binds preferentially to the target of interest or binds with greater affinity to the target than to other molecules. For example, an antibody will bind to a molecule comprising an epitope for which it is specific and not to unrelated epitopes. In some embodiments, the affinity reagent may be an antibody, i.e. an antibody that is specific for TCRβ, CD4, or CD25. In some embodiments, the affinity reagent may be a specific receptor or ligand for TCRβ, CD4, or CD25, e.g. a peptide ligand and receptor; effector and receptor molecules, and the like. In some embodiments, multiple affinity reagents specific for TCRβ, CD4, or CD25 may be used.

Antibodies and T cell receptors that find use as affinity reagents may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art. Of particular interest is the use of labeled antibodies as affinity reagents. Conveniently, these antibodies are conjugated with a label for use in separation. Labels include magnetic beads, which allow for direct separation; biotin, which can be removed with avidin or streptavidin bound to a support; fluorochromes, which can be used with a fluorescence activated cell sorter; or the like, to allow for ease of separation of the particular cell type. Fluorochromes that find use include phycobiliproteins, e.g. phycoerythrin and allophycocyanins, fluorescein and Texas red. Frequently each antibody is labeled with a different fluorochrome, to permit independent sorting for each marker.

The subject initial population of leukocytes are contacted with the affinity reagent(s) and incubated for a period of time sufficient to bind the available cell surface antigens. The incubation will usually be at least about 5 minutes and usually less than about 60 minutes. It is desirable to have a sufficient concentration of antibodies in the reaction mixture, such that the efficiency of the separation is not limited by lack of antibody. The appropriate concentration is determined by titration, but will typically be a dilution of antibody into the volume of the cell suspension that is about 1:50 (i.e., 1 part antibody to 50 parts reaction volume), about 1:100, about 1:150, about 1:200, about 1:250, about 1:500, about 1:1000, about 1:2000, or about 1:5000. The medium in which the cells are suspended will be any medium that maintains the viability of the cells. A preferred medium is phosphate buffered saline containing from 0.1 to 0.5% BSA or 1-4% goat serum. Various media are commercially available and may be used according to the nature of the cells, including Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with fetal calf serum, BSA, HSA, goat serum etc.

The cells in the contacted population that become labeled by the affinity reagent, i.e. the Tregs, are selected for by any convenient affinity separation technique, e.g. as described above or as known in the art. Following separation, the separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum.

Compositions that are highly enriched for regulatory T cells are achieved in this manner. By “highly enriched”, it is meant that the regulatory T cells will be 70% or more, 75% or more, 80% or more, 85% or more, 90% or more of the cell composition, for example, about 95% or more, or 98% or more of the cell composition. In other words, the composition may be a substantially pure composition of regulatory T cells.

Regulatory T cells produced by the methods described herein may be used immediately. Alternatively, the regulatory T cells may be frozen at liquid nitrogen temperatures and stored for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells.

The regulatory T cells may be cultured in vitro under various culture conditions. Culture medium may be liquid or semi-solid, e.g. containing agar, methylcellulose, etc. The cell population may be conveniently suspended in an appropriate nutrient medium, such as Iscove's modified DMEM or RPMI 1640, normally supplemented with fetal calf serum (about 5-10%), L-glutamine, a thiol, particularly 2-mercaptoethanol, and antibiotics, e.g. penicillin and streptomycin. The culture may contain growth factors to which the regulatory T cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

The regulatory T cells may be used in any of a number of applications, including research applications, e.g. experiments to better understand the mechanism of action of regulatory T cells, experiments to identify candidate agents that promote the activity of regulatory T cells, etc., and medical applications, e.g. to treat autoimmune or rheumatoid diseases as discussed above and described further below.

If used in medical applications, the regulatory T cells may be provided alone or with a suitable substrate or matrix, e.g. to support their growth and/or organization in the tissue to which they are being transplanted. Usually, at least 1×10³ cells will be administered, for example 5×10³ cells, 1×10⁴ cells, 5×10⁴ cells, 1×10⁵ cells, 1×10⁶ cells or more. The cells may be introduced to the subject via any of the following routes: parenteral, subcutaneous, intravenous, intracranial, intraspinal, intraocular, or into spinal fluid. The cells may be introduced by injection, catheter, or the like. Examples of methods for local delivery, that is, delivery to the site of injury, include, e.g. through an Ommaya reservoir, e.g. for intrathecal delivery (see e.g. U.S. Pat. Nos. 5,222,982 and 5,385,582, incorporated herein by reference); by bolus injection, e.g. by a syringe, e.g. into a joint; by continuous infusion, e.g. by cannulation, e.g. with convection (see e.g. US Application No. 20070254842, incorporated here by reference); or by implanting a device upon which the cells have been reversably affixed (see e.g. US Application Nos. 20080081064 and 20090196903, incorporated herein by reference).

The number of administrations of treatment to a subject may vary. Introducing the regulatory T cells into the subject may be a one-time event; but in certain situations, such treatment may elicit improvement for a limited period of time and require an on-going series of repeated treatments. In other situations, multiple administrations of the regulatory T cells may be required before an effect is observed. The exact protocols depend upon the disease or condition, the stage of the disease and parameters of the individual subject being treated.

In other aspects of the invention, the SAA composition is employed to induce regulatory T cell proliferation in vivo, e.g. to treat a subject with an autoimmune or rheumatoid disease. Any subject with an autoimmune or rheumatoid disease may be treated in these methods. For example, the subject may be a neonate, a juvenile, or an adult. Of particular interest are mammalian subjects. Mammalian species that may be treated with the present methods include canines and felines; equines; bovines; ovines; etc. and primates, particularly humans. Animal models, particularly small mammals, e.g. murine, lagomorpha, etc. may be used for experimental investigations. Subjects with an autoimmune or rheumatoid disease may be identified using criteria known in the art and described above. Autoimmune or rheumatoid diseases of interest include, but are not limited to rheumatoid arthritis, juvenile idiopathic arthritis, systemic lupus erythematosus, spondyloarthropathy, psoriatic arthritis, Kawasaki disease, Sjogren's syndrome, sarcoidosis, multiple sclerosis, type 1 diabetes mellitus, graft versus host disease, transplant rejection, ulcerative colitis, and Crohn's disease.

In these in vivo embodiments, the SAA composition is administered directly to the individual with the autoimmune or rheumatoid disease. SAA compositions may be administered by any of a number of well-known methods in the art for the administration of peptides, small molecules and nucleic acids to a subject. The SAA composition can be incorporated into a variety of formulations. More particularly, the SAA compositions of the present invention can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the SAA composition can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release.

For some conditions, particularly central nervous system conditions, it may be necessary to formulate agents to cross the blood-brain barrier (BBB). One strategy for drug delivery through the blood-brain barrier (BBB) entails disruption of the BBB, either by osmotic means such as mannitol or leukotrienes, or biochemically by the use of vasoactive substances such as bradykinin. The potential for using BBB opening to target specific agents to brain tumors is also an option. A BBB disrupting agent can be co-administered with the therapeutic compositions of the invention when the compositions are administered by intravascular injection. Other strategies to go through the BBB may entail the use of endogenous transport systems, including Caveolin-1 mediated transcytosis, carrier-mediated transporters such as glucose and amino acid carriers, receptor-mediated transcytosis for insulin or transferrin, and active efflux transporters such as p-glycoprotein. Active transport moieties may also be conjugated to the therapeutic compounds for use in the invention to facilitate transport across the endothelial wall of the blood vessel. Alternatively, drug delivery of therapeutics agents behind the BBB may be by local delivery, for example by intrathecal delivery, e.g. through an Ommaya reservoir (see e.g. U.S. Pat. Nos. 5,222,982 and 5,385,582, incorporated herein by reference); by bolus injection, e.g. by a syringe, e.g. intravitreally or intracranially; by continuous infusion, e.g. by cannulation, e.g. with convection (see e.g. US Application No. 20070254842, incorporated here by reference); or by implanting a device upon which the agent has been reversably affixed (see e.g. US Application Nos. 20080081064 and 20090196903, incorporated herein by reference).

The calculation of the effective amount or effective dose of SAA composition to be administered is within the skill of one of ordinary skill in the art, and will be routine to those persons skilled in the art. Needless to say, the final amount to be administered will be dependent upon the route of administration and upon the nature of the disorder or condition that is to be treated.

For inclusion in a medicament, the SAA composition may be obtained from a suitable commercial source. As a general proposition, the total pharmaceutically effective amount of the SAA composition administered parenterally per dose will be in a range that can be measured by a dose response curve.

SAA-based therapies, i.e. preparations of SAA compositions to be used for therapeutic administration must be sterile. In the case of cell preparations, sterility is readily accomplished by maintaining sterile techniques. In the case of preparations of SAA compositions, sterility is readily accomplished by filtration through sterile filtration membranes (e.g., 0.2 μm membranes). Therapeutic compositions generally are placed into a container having a sterile access port, for example, an intravenous solution bag or vial having a stopper pierceable by a hypodermic injection needle. The SAA-based therapies may be stored in unit or multi-dose containers, for example, sealed ampules or vials, as an aqueous solution or as a lyophilized formulation for reconstitution. As an example of a lyophilized formulation, 10-mL vials are filled with 5 ml of sterile-filtered 1% (w/v) aqueous solution of compound, and the resulting mixture is lyophilized. The infusion solution is prepared by reconstituting the lyophilized compound using bacteriostatic Water-for-Injection.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices are preferred.

The data obtained from cell culture and/or animal studies can be used in formulating a range of dosages for humans. The dosage of the active ingredient typically lines within a range of circulating concentrations that include the ED50 with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The effective amount of a therapeutic composition to be given to a particular patient will depend on a variety of factors, several of which will differ from patient to patient. A competent clinician will be able to determine an effective amount of a therapeutic agent to administer to a patient to halt or reverse the progression the disease condition as required. Utilizing LD50 animal data, and other information available for the agent, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

More general methods of formulating and administering drug compositions to an individual are discussed in greater detail below.

Therapeutic administration of SAA compositions can include administration as a part of a therapeutic regimen that may or may not be in conjunction with additional standard anti-autoimmune disease therapeutics, including but not limited to anti-inflammatory therapy, immunosuppressant therapy, antibody therapy, and the like. Additionally or alternatively, therapeutic administration of SAA compositions can be post-therapeutic treatment of the subject with an anti-autoimmune disease therapy, where the anti-autoimmune disease therapy can be, for example, anti-inflammatory therapy, immunosuppressant therapy, antibody therapy, and the like. For example. SAA compositions may be provided in combination with an immunosuppressant, e.g., corticosteroids (such as prednisone), or nonsteroid drugs such as azathioprine, cyclophosphamide, mycophenolate, sirolimus, or tacrolimus. As another example, SAA compositions may be provided in combination with an antibody-based therapy, e.g. anti-CD52, anti-TNFα, etc.

Manipulating Proteins, DNA, and RNA

According to the central dogma of molecular biology, DNA is transcribed into RNA, and RNA is translated into protein; one gene makes one protein. DNA, or deoxyribonucleic acid, is a polynucleotide formed from covalently linked deoxyribonucleotide units. RNA, or ribonucleic acid, is a polynucleotide formed from covalently linked ribonucleotide units. Protein is a linear polymer of amino acids linked together by peptide bonds.

Isolating Cells and Growing them in Culture

Although the organelles and large molecules in a cell can be visualized with microscopes, understanding how these components function requires a detailed biochemical analysis. Most biochemical procedures require that large numbers of cells be physically disrupted to gain access to their components. If the sample is a piece of tissue, composed of different types of cells, heterogeneous cell populations will be mixed together. To obtain as much information as possible about the cells in a tissue, biologists have developed ways of dissociating cells from tissues and separating them according to type. These manipulations result in a relatively homogeneous population of cells that can then be analyzed—either directly or after their number has been greatly increased by allowing the cells to proliferate in culture.

Cells can be Isolated from Intact Tissues

Intact tissues provide the most realistic source of material, as they represent the actual cells found within the body. The first step in isolating individual cells is to disrupt the extracellular matrix and cell-cell junctions that hold the cells together. For this purpose, a tissue sample is typically treated with proteolytic enzymes (such as trypsin and collagenase) to digest proteins in the extracellular matrix and with agents (such as ethylenediaminetetraacetic acid, or EDTA) that bind, or chelate, the Ca2+ on which cell-cell adhesion depends. The tissue can then be teased apart into single cells by gentle agitation. For some biochemical preparations, the tissue or organ need not be separated into cell types. In other cases, enrichment for a specific cell type of interest is required. Several approaches are used to separate the different cell types from a mixed cell suspension. The most general cell-separation technique uses an antibody coupled to a fluorescent dye to label specific cells. An antibody is chosen that specifically binds to the surface of only one cell type in the tissue. The labeled cells can then be separated from the unlabeled ones in an electronic fluorescence-activated cell sorter. In this machine, individual cells traveling single file in a fine stream pass through a laser beam, and the fluorescence of each cell is rapidly measured. A vibrating nozzle generates tiny droplets, most containing either one cell or no cells. The droplets containing a single cell are automatically given a positive or a negative charge at the moment of formation, depending on whether the cell they contain is fluorescent; they are then deflected by a strong electric field into an appropriate container. Occasional clumps of cells, detected by their increased light scattering, are left uncharged and are discarded into a waste container. Such machines can accurately select 1 fluorescent cell from a pool of 1000 unlabeled cells and sort several thousand cells each second.

Selected cells can also be obtained by carefully dissecting them from thin tissue slices that have been prepared for microscopic examination. In one approach, a issue section is coated with a thin plastic film and a region containing the cells of interest is irradiated with a focused pulse from an infrared laser. This light pulse melts a small circle of the film, binding the cells underneath. These captured cells are then removed for further analysis. The technique, called laser capture microdissection, can be used to separate and analyze cells from different areas of a tumor, allowing their properties or molecular composition to be compared with neighboring normal cells. A related method uses a laser beam to directly cut out a group of cells and catapult them into an appropriate container for future analysis. A uniform population of cells obtained by any of these or other separation methods can be used directly for biochemical analysis. After breaking open the cells by mechanical disruption, detergents, and other methods, cytoplasm or individual organelles can be extracted and then specific molecules purified.

Cells can be Grown in Culture

The complexity of intact tissues and organs is an inherent disadvantage when trying to extract certain materials. Cells grown in culture provide a more homogeneous population of cells with which to work. Given appropriate surroundings, most plant and animal cells can live, multiply, and even express differentiated properties in a tissue-culture dish. The cells can be watched continuously under the microscope or analyzed biochemically, and the effects of adding or removing specific molecules, such as hormones or growth factors, can be systematically explored. In addition, by mixing two cell types, the interactions between one cell type and another can be studied.

Experiments performed on cultured cells are sometimes said to be carried out in vitro (literally, “in glass”) to contrast them with experiments using intact organisms, which are said to be carried out in vivo (literally, “in the living organism”). These terms can be confusing, however, because they are often used in a very different sense by biochemists. In the biochemistry lab, in vitro refers to actions carried out in a test tube in the absence of living cells, whereas in vivo refers to any reaction taking place inside a living cell, even if that cell is growing in culture.

Cultures are most commonly made from suspensions of cells dissociated from tissues using the methods described earlier. Unlike bacteria, most tissue cells are not adapted to living suspended in fluid and require a solid surface on which to grow and divide. For cell cultures this support is usually provided by the surface of a plastic tissue-culture dish. Cells vary in their requirements, however, and many do not proliferate or differentiate unless the culture dish is coated with materials that cells like to adhere to, such as polylysine or extracellular matrix components.

Cultures prepared directly from the tissues of an organism are called primary cultures. These can be made with or without an initial fractionation step to separate different cell types. In most cases, cells in primary cultures can be removed from the culture dish and recultured repeatedly in so-called secondary cultures; in this way, they can be repeatedly subcultured (passaged) for weeks or months. Such cells often display many of the differentiated properties appropriate to their origin: fibroblasts continue to secrete collagen; cells derived from embryonic skeletal muscle fuse to form muscle fibers that contract spontaneously in the culture dish; nerve cells extend axons that are electrically excitable and make synapses with other nerve cells; and epithelial cells form extensive sheets with many of the properties of an intact epithelium. Because these properties are maintained in culture, they are accessible to study in ways that are often not possible in intact tissues.

Cell culture is not limited to animal cells. When a piece of plant tissue is cultured in a sterile medium containing nutrients and appropriate growth regulators, many of the cells are stimulated to proliferate indefinitely in a disorganized manner, producing a mass of relatively undifferentiated cells called a callus. If the nutrients and growth regulators are carefully manipulated, one can induce the formation of a shoot and then root apical meristems within the callus, and in many species, regenerate a whole new plant. Similar to animal cells, callus cultures can be mechanically dissociated into single cells, which will grow and divide as a suspension culture.

Eukaryotic Cell Lines are a Widely Used Source of Homogeneous Cells

The cell cultures obtained by disrupting tissues tend to suffer from a problem—eventually the cells die. Most vertebrate cells stop dividing after a finite number of cell divisions in culture, a process called replicative cell senescence. Normal human fibroblasts, for example, typically divide only 25-40 times in culture before they stop. In these cells, the limited proliferation capacity reflects a progressive shortening and uncapping of the cell's telomeres, the repetitive DNA sequences and associated proteins that cap the ends of each chromosome. Human somatic cells in the body have turned off production of the enzyme, called telomerase, which normally maintains the telomeres, which is why their telomeres shorten with each cell division. Human fibroblasts can often be coaxed to proliferate indefinitely by providing them with the gene that encodes the catalytic subunit of telomerase; in this case, they can be propagated as an “immortalized” cell line.

Some human cells, however, cannot be immortalized by this trick. Although their telomeres remain long, they still stop dividing after a limited number of divisions because the culture conditions eventually activate cell-cycle check-point mechanisms that arrest the cell cycle—a process sometimes called “culture shock.” In order to immortalize these cells, one has to do more than introduce telomerase. One must also inactivate the checkpoint mechanisms. This can be done by introducing certain cancer-promoting oncogenes, such as those derived from tumor viruses. Unlike human cells, most rodent cells do not turn off production of telomerase and therefore their telomeres do not shorten with each cell division. Therefore, if culture shock can be avoided, some rodent cell types will divide indefinitely in culture. In addition, rodent cells often undergo genetic changes in culture that inactivate their checkpoint mechanisms, thereby spontaneously producing immortalized cell lines.

Cell lines can often be most easily generated from cancer cells, but these cultures differ from those prepared from normal cells in several ways, and are referred to as transformed cell lines. Transformed cell lines often grow without attaching to a surface, for example, and they can proliferate to a much higher density in a culture dish. Similar properties can be induced experimentally in normal cells by transforming them with a tumor-inducing virus or chemical. The resulting transformed cell lines can usually cause tumors if injected into a susceptible animal. Both transformed and nontransformed cell lines are extremely useful in cell research as sources of very large numbers of cells of a uniform type, especially since they can be stored in liquid nitrogen at −196° C. for an indefinite period and retain their viability when thawed. It is important to keep in mind, however, that the cells in both types of cell lines nearly always differ in important ways from their normal progenitors in the tissues from which they were derived.

Some widely used cell lines are as follows, listing cell line and cell type (and origin): 3T3, fibroblast (mouse); BHK, fibroblast (Syrian hamster); MDCK, epithelial cell (dog); HeLa, epithelial cell (human); PtK1, epithelial cell (rat kangaroo); L6, myoblast (rat); PC12, chromaffin cell (rat); SP2, plasma cell (mouse); COS, kidney (monkey); 293 kidney (human, transformed with adenovirus); CHO, ovary (Chinese hamster); DT40, lymphoma cell for efficient targeted recombination (chick); R1, embryonic stem cell (mouse); E14.1, embryonic stem cell (mouse); H1, H9, embryonic stem cell (human); S2, macrophage-like cell (Drosophila); BY2, undifferentiated meristematic cell (tobacco).

Hybridoma Cell Lines are Factories that Produce Monoclonal Antibodies

An antibody, also called an immunoglobulin (Ig), is a protein produced by cells of the immune system in response to an antigen. Antibodies are particularly useful tools for cell biology. Their great specificity allows precise visualization of selected proteins among the many thousands that each cell typically produces. Antibodies are often produced by inoculating animals with the protein of interest and subsequently isolating the antibodies specific to that protein from the serum of the animal. However, only limited quantities of antibodies can be obtained from a single inoculated animal, and the antibodies produced will be a heterogeneous mixture of antibodies that recognize a variety of different determinants on a macromolecule that differs from animal to animal. Moreover, antibodies specific for the antigen will constitute only a fraction of the antibodies found in the serum. An alternative technology, which allows the production of an infinite quantity of identical antibodies and greatly increases the specificity and convenience of antibody-based methods, is the production of monoclonal antibodies by hybridoma cell lines.

This technology has facilitated the production of antibodies for use as tools in cell biology, as well as for the diagnosis and treatment of certain diseases. The procedure requires hybrid cell technology, and it involves propagating a clone of cells from a single antibody-secreting B lymphocyte to obtain a homogeneous preparation of antibodies in large quantities. B lymphocytes normally have a limited life-span in culture, but individual antibody-producing B lymphocytes from an immunized mouse or rat, when fused with cells derived from a transformed B lymphocyte cell line, can give rise to hybrids that have both the ability to make a particular antibody and the ability to multiply indefinitely in culture. These hybridomas are propagated as individual clones, each of which provides a permanent and stable source of a single type of monoclonal antibody. Each type of monoclonal antibody recognizes a single determinant of an antigen—for example, a particular cluster of five or six amino acid side chains on the surface of a protein. Their uniform specificity makes monoclonal antibodies much more useful than conventional antisera for most purposes.

Hybridomas are prepared that secrete monoclonal antibodies against a particular antigen by immunizing a mouse with antigen X and fusing the cells that make antibodies (including the cell making anti-X antibody) obtained from the spleen with a mutant cell line derived from a tumor of B lymphocytes. The selective growth medium used after the cell fusion step contains an inhibitor (aminopterin) that blocks the normal biosynthetic pathways by which nucleotides are made. The cells must therefore use a bypass pathway to synthesize their nucleic acids. This pathway is defective in the mutant cell line derived from the B cell tumor, but it is intact in the normal cells obtained from the immunized mouse. Nevertheless, the normal B lymphocytes will die after a few days in culture. Because neither cell type used for the initial fusion can survive and proliferate on its own, only the hybridoma cells do so. The hybridoma cells are cloned by limiting dilution, the supernatants tested for anti-X antibodies, and positive clones selected that provide a continuing source of anti-X antibody.

An important advantage of the hybridoma technique is that monoclonal antibodies can be made against molecules that constitute only a minor component of a complex mixture. In an ordinary antiserum made against such a mixture, the proportion of antibody molecules that recognize the minor component would be too small to be useful. But if the B lymphocytes that produce the various components of this antiserum are made into hybridomas, it becomes possible to screen individual hybridoma clones from the large mixture to select one that produces the desired type of monoclonal antibody and to propagate the selected hybridoma indefinitely so as to produce that antibody in unlimited quantities. In principle, therefore, a monoclonal antibody can be made against any protein in a biological sample. Once an antibody has been made, it can be used to localize the protein in cells and tissues, to follow its movement, and to purify the protein of interest.

Purifying Proteins

The challenge of isolating a single type of protein from the thousands of other proteins present in a cell is a formidable one, but must be overcome in order to produce purified proteins. Recombinant DNA technology can enormously simplify this task by “tricking” cells into producing large quantities of a given protein, thereby making its purification a little easier. Whether the source of the protein is an engineered cell or a natural tissue, a purification procedure usually starts with subcellular fractionation to reduce the complexity of the material, and is then followed by purification steps of increasing specificity.

Cells can be Separated into their Component Fractions

In order to purify a protein, it must first be extracted from inside the cell. Cells may be broken up in various ways: they can be subjected to osmotic shock or ultrasonic vibration, forced through a small orifice, or ground up in a blender. These procedures break many of the membranes of the cell (including the plasma membrane endoplasmic reticulum) into fragments that immediately reseal to form small closed vesicles. If carefully carried out, however, the disruption procedures leave organelles such as nuclei, mitochondria, the Golgi apparatus, lysosomes, and peroxisomes largely intact. The suspension of cells is thereby reduced to a thick slurry called a homogenate or extract) that contains a variety of membrane-enclosed organelles, each with a distinctive size, charge and density. Provided that the homogenization medium has been carefully chosen (by trial and error for each organelle), the various components—including the vesicles derived from the endoplasmic reticulum, called microsomes—retain most of their original biochemical properties.

The different components of the homogenate must then be separated. Such cell fractionations became possible only after the commercial development of an instrument known as the preparative ultracentrifuge, which rotates extracts of broken cells at high speeds. This treatment separates cell components by size and density: in general, the largest units experience the largest centrifugal force and move the most rapidly. At relatively low speed, large components such as nuclei sediment to form a pellet at the bottom of the centrifuge tube; at slightly higher speed, a pellet of mitochondria is deposited; and at even higher speeds and with longer periods of centrifugation, first the small closed vesicles and then the ribosomes can be collected. All of these fractions are impure, but many of the contaminants can be removed by resuspending the pellet and repeating the centrifugation procedure several times.

Centrifugation is the first step in most fractionations, but it separates only components that differ greatly in size. A finer degree of separation can be achieved by layering the homogenate in a thin band on top of a dilute salt solution that fills a centrifuge tube. When centrifuged, the various components in the mixture move as a series of distinct bands through the salt solution, each at a different rate, in a process called velocity sedimentation. For the procedure to work effectively, the bands must be protected from convective mixing, which would normally occur whenever a denser solution (for example, one containing organelles) finds itself on top of a lighter one (the salt solution). This is achieved by augmenting the solution in the tube with a shallow gradient of sucrose prepared by a special mixing device. The resulting density radient—with the dense end at the bottom of the tube—keeps each region of the salt solution denser than any solution above it, and it thereby prevents convective mixing from distorting the separation.

When sedimented through such dilute sucrose gradients, different cell components separate into distinct bands that can be collected individually. The relative rate at which each component sediments depends primarily on its size and shape—normally being described in terms of its sedimentation coefficient, or S value. Present-day ultracentrifuges rotate at speeds of up to 80,000 rpm and produce forces as high as 500,000 times gravity. These enormous forces drive even small macromolecules, such as tRNA molecules and simple enzymes, to sediment at an appreciable rate and allow them to be separated from one another by size. The ultracentrifuge is also used to separate cell components on the basis of their buoyant density, independently of their size and shape. In this case the sample is sedimented through a steep density gradient that contains a very high concentration of sucrose or cesium chloride. Each cell component begins to move down the gradient, but it eventually reaches a position where the density of the solution is equal to its own density. At this point the component floats and can move no farther. A series of distinct bands is thereby produced in the centrifuge tube, with the bands closest to the bottom of the tube containing the components of highest buoyant density. This method, called equilibrium sedimentation, is so sensitive that it can separate macromolecules that have incorporated heavy isotopes, such as 13C or 15N, from the same macromolecules that contain the lighter, common isotopes (12C or 14N).

Cell Extracts Provide Accessible Systems to Study Cell Functions

Cell extracts isolated in the ultracentrifuge have contributed to our understanding of cell functions. They have played a good role in the study of cell processes. Cell extracts also provide, in principle, the starting material for the separation of proteins. Proteins Can Be Separated by Chromatography Proteins are often fractionated by column chromatography, in which a mixture of proteins in solution is passed through a column containing a porous solid matrix. The different proteins are retarded to different extents by their interaction with the matrix, such as cellulose, and they can be collected separately as they flow out of the bottom of the column. Depending on the choice of matrix, proteins can be separated according to their charge (ion-exchange chromatography), their hydrophobicity (hydrophobic chromatography), their size (gel-filtration chromatography), or their ability to bind to particular small molecules or to other macromolecules (affinity chromatography).

Many types of matrices are commercially available. Ion exchange columns are packed with small beads that carry either a positive or a negative charge, so that proteins are fractionated according to the arrangement of charges on their surface. Hydrophobic columns are packed with beads from which hydrophobic side chains protrude, selectively retarding proteins with exposed hydrophobic regions. Gel filtration columns, which separate proteins according to their size, are packed with tiny porous beads: molecules that are small enough to enter the pores linger inside successive beads as they pass down the column, while larger molecules remain in the solution flowing between the beads and therefore move more rapidly, emerging from the column first.

Inhomogeneities in the matrices (such as cellulose), which cause an uneven flow of solvent through the column, limit the resolution of conventional column chromatography. Special chromatography resins (usually silica-based) composed of tiny spheres (3-10 μm in diameter) can be packed with a special apparatus to form a uniform column bed. Such high-performance liquid chromatography (HPLC) columns attain a high degree of resolution. In HPLC, the solutes equilibrate very rapidly with the interior of the tiny spheres, and so solutes with different affinities for the matrix are efficiently separated from one another even at very fast flow rates. HPLC is therefore the method of choice for separating many proteins and small molecules.

Affinity Chromatography Exploits Specific Binding Sites on Proteins

If one starts with a complex mixture of proteins, the types of column chromatography just discussed do not produce very highly purified fractions: a single passage through the column generally increases the proportion of a given protein in the mixture no more than twentyfold. Because most individual proteins represent less than 1/1000 of the total cell protein, it is usually necessary to use several different types of columns in succession to attain sufficient purity. A more efficient procedure, known as affinity chromatography, takes advantage of the biologically important binding interactions that occur on protein surfaces. If a substrate molecule is covalently coupled to an inert matrix such as a polysaccharide bead, the enzyme that operates on that substrate will often be specifically retained by the matrix and can then be eluted (washed out) in nearly pure form. Likewise, short DNA oligonucleotides of a specifically designed sequence can be immobilized in this way and used to purify DNA-binding proteins that normally recognize this sequence of nucleotides in chromosomes. Alternatively, specific antibodies can be coupled to a matrix to purify protein molecules recognized by the antibodies. Because of the great specificity of all such affinity columns, 1000- to 10,000-fold purifications can sometimes be achieved in a single pass.

Three types of matrices commonly used for chromatography can be compared as follows. In ion-exchange chromatography, the insoluble matrix carries ionic charges that retard the movement of molecules of opposite charge. Matrices used for separating proteins include diethylaminoethylcellulose (DEAE-cellulose), which is positively charged, and carboxymethylcellulose (CM-cellulose) and phosphocellulose, which are negatively charged. Analogous-matrices based on agarose or other polymers are also frequently used. The strength of the association between the dissolved molecules and the ion-exchange matrix depends on both the ionic strength and the pH of the solution that is passing down the column, which may therefore be varied systematically to achieve an effective separation. In gel-filtration chromatography, the matrix is inert but porous. Molecules that are small enough to penetrate into the matrix are thereby delayed and travel more slowly through the column than larger molecules that cannot penetrate. Beads of cross-linked polysaccharide (dextran, agarose or acrylamide) are available commercially in a wide range of pore sizes, making them suitable for the fractionation of molecules of various molecular weights, from less than 500 daltons to more than 5×10̂6 daltons. Affinity chromatography uses an insoluble matrix that is covalently linked to a specific ligand, such as an antibody molecule or an enzyme substrate that will bind a specific protein. Enzyme molecules that bind to immobilized substrates on such columns can be eluted with a concentrated solution of the free form of the substrate molecule, while molecules that bind to immobilized antibodies can be eluted by dissociating the antibody-antigen complex with concentrated salt solutions or solutions of high or low pH. High degrees of purification can be achieved in a single pass through an affinity column.

Genetically-Engineered Tags Provide an Easy Way to Purify Proteins

Using recombinant DNA methods, a gene can be modified to produce its protein with a special recognition tag attached to it, so as to make subsequent purification of the protein by affinity chromatography simple and rapid. Often the recognition tag is itself an antigenic determinant, or epitope, which can be recognized by a highly specific antibody. The antibody can then be used both to localize the protein in cells and to purify it. Other types of tags are specifically designed for protein purification. For example, the amino acid histidine binds to certain metal ions, including nickel and copper. If genetic engineering techniques are used to attach a short string of histidines to one end of a protein, the slightly modified protein can be retained selectively on an affinity column containing immobilized nickel ions. Metal affinity chromatography can thereby be used to purify the modified protein from a complex molecular mixture. In other cases, an entire protein is used as the recognition tag. When cells are engineered to synthesize the small enzyme glutathione S-transferase (GST) attached to a protein of interest, the resulting fusion protein can be purified from the other contents of the cell with an affinity column containing glutathione, a substrate molecule that binds specifically and tightly to GST. If the purification is carried out under conditions that do not disrupt protein-protein interactions, the fusion protein can be isolated in association with the proteins it interacts with inside the cell.

As a further refinement of purification methods using recognition tags, an amino acid sequence that forms a cleavage site for a highly specific proteolytic enzyme can be engineered between the protein of choice and the recognition tag. Because the amino acid sequences at the cleavage site are very rarely found by chance in proteins, the tag can later be cleaved off without destroying the purified protein.

This type of specific cleavage is used in an especially powerful purification methodology known as tandem affinity purification tagging (tap-tagging). Here, one end of a protein is engineered to contain two recognition tags that are separated by a rotease cleavage site. The tag on the very end of the construct is chosen to bind irreversibly to an affinity column, allowing the column to be washed extensively to remove all contaminating proteins. Protease cleavage then releases the protein, which is then further purified using the second tag. Because this two-step strategy provides a n especially high degree of protein purification with relatively little effort, it is used extensively in cell biology.

Purified Cell-Free Systems Facilitate the Dissection of Molecular Functions

It is advantageous to study biological processes free from all of the complex side reactions that occur in a living cell by using purified cell-free systems. To make this possible, cell homogenates are fractionated with the aim of purifying each of the individual macromolecules that are needed to catalyze a biological process of interest. For example, the experiments to decipher the mechanisms of protein synthesis began with a cell homogenate that could translate RNA molecules to produce proteins. Fractionation of this homogenate, step by step, produced in turn the ribosomes, tRNAs, and various enzymes that together constitute the protein-synthetic machinery. Once individual pure components were available, each could be added or withheld separately to define its exact role in the overall process.

Analyzing Proteins

Proteins perform most processes in cells: they catalyze metabolic reactions, use nucleotide hydrolysis to do mechanical work, and serve as the major structural elements of the cell. The great variety of protein structures and functions has stimulated the development of a multitude of techniques to study them.

Proteins can be Separated by SDS Polyacrylamide-Gel Electrophoresis

Proteins usually possess a net positive or negative charge, depending on the mixture of charged amino acids they contain. An electric field applied to a solution containing a protein molecule causes the protein to migrate at a rate that depends on its net charge and on its size and shape. The most popular application of this property is SDS polyacrylamide-gel electrophoresis (SDS-PAGE). It uses a highly cross-linked gel of polyacrylamide as the inert matrix through which the proteins migrate. The gel is prepared by polymerization of monomers; the pore size of the gel can be adjusted so that it is small enough to retard the migration of the protein molecules of interest. The proteins themselves are not in a simple aqueous solution but in one that includes a powerful negatively charged detergent, sodium dodecyl sulfate, or SDS. Because this detergent binds to hydrophobic regions of the protein molecules, causing them to unfold into extended polypeptide chains, the individual protein molecules are released from their associations with other proteins or lipid molecules and rendered freely soluble in the detergent solution. In addition, a reducing agent such as β-mercaptoethanol is usually added to break any S-S linkages in the proteins, so that all of the constituent polypeptides in multisubunit proteins can be analyzed separately.

What happens when a mixture of SDS-solubilized proteins is run through a slab of polyacrylamide gel? Each protein molecule binds large numbers of the negatively charged detergent molecules, which mask the protein's intrinsic charge and cause it to migrate toward the positive electrode when a voltage is applied. Proteins of the same size tend to move through the gel with similar speeds because (1) their native structure is completely unfolded by the SDS, so that their shapes are the same, and (2) they bind the same amount of SDS and therefore have the same amount of negative charge. Larger proteins, with more charge, are subjected to larger electrical forces and also to a larger drag. In free solution, the two effects would cancel out, but, in the mesh of the polyacrylamide gel, which acts as a molecular sieve, large proteins are retarded much more than small ones. As a result, a complex mixture of proteins is fractionated into a series of discrete protein bands arranged in order of molecular weight. The major proteins are readily detected by staining the proteins in the gel with a dye such as Coomassie blue. Even minor proteins are seen in gels treated with a silver or gold stain, so that as little asng of protein can be detected in a band.

SDS-PAGE is widely used because it can separate all types of proteins, including those that are normally insoluble in water—such as the many proteins in membranes. And because the method separates polypeptides by size, it provides information about the molecular weight and the subunit composition of proteins. A photograph of a Coomasie-stained gel is handy for memorializing an analysis of each of the successive stages in the purification of a protein.

Specific Proteins can be Detected by Blotting with Antibodies

A specific protein can be identified after its fractionation on a polyacrylamide gel by exposing all the proteins present on the gel to a specific antibody that has been coupled to a radioactive isotope, to an easily detectable enzyme, or to a fluorescent dye. For convenience, this procedure is normally carried out after transferring (by “blotting”) all of the separated proteins present in the gel onto a sheet of nitrocellulose paper or nylon membrane. Placing the membrane over the gel and driving the proteins out of the gel with a strong electric field transfers the protein onto the membrane. The membrane is then soaked in a solution of labeled antibody to reveal the protein of interest. This method of detecting proteins is called Western blotting, or immunoblotting.

Mass Spectrometry Provides a Method for Identifying Unknown Proteins

A frequent problem in cell biology and biochemistry is the identification of a protein or collection of proteins that has been obtained by one of the purification procedures for proteins. Because the genome sequences of most common experimental organisms are now known, catalogues of all the proteins produced in those organisms are available. The task of identifying an unknown protein (or collection of unknown proteins) thus reduces to matching some of the amino acid sequences present in the unknown sample with known catalogued genes. This task is now performed almost exclusively by using mass spectrometry in conjunction with computer searches of databases.

Charged particles have very precise dynamics when subjected to electrical and magnetic fields in a vacuum. Mass spectrometry exploits this principle to separate ions according to their mass-to-charge ratio. It is an enormously sensitive technique. It requires very little material and is capable of determining the precise mass of intact proteins and of peptides derived from them by enzymatic or chemical cleavage. Masses can be obtained with great accuracy, often with an error of less than one part in a million. The most commonly used form of the technique is called matrix-assisted laser desorption ionization-time-of-flight spectrometry (MALDI-TOF). In this approach, the proteins in the sample are first broken into short peptides. These peptides are mixed with an organic acid and then dried onto a metal or ceramic slide. A laser then blasts the sample, ejecting the peptides from the slide in the form of an ionized gas, in which each molecule carries one or more positive charges. The ionized peptides are accelerated in an electric field and fly toward a detector. Their mass and charge determines the time it takes them to reach the detector: large peptides move more slowly, and more highly charged molecules move more quickly. By analyzing those ionized peptides that bear a single charge, the precise masses of peptides present in the original sample can be determined. MALDI-TOF can also be used to accurately measure the mass of intact proteins as large as 200,000 daltons. This information is then used to search genomic databases, in which the masses of all proteins and of all their predicted peptide fragments have been tabulated from the genomic sequences of the organism. An unambiguous match to a particular open reading frame can sometimes be made by knowing the mass of only a few peptides derived from a given protein.

MALDI-TOF provides accurate molecular weight measurements for proteins and peptides. Moreover, by employing two mass spectrometers in tandem (an arrangement known as MS/MS), it is possible to directly determine the amino acid sequences of individual peptides in a complex mixture. As described above, the protein sample is first broken down into smaller peptides, which are separated from each other by mass spectrometry. Each peptide is then further fragmented through collisions with high-energy gas atoms. This method of fragmentation preferentially cleaves the peptide bonds, generating a ladder of fragments, each differing by a single amino acid. The second mass spectrometer then separates these fragments and displays their masses. The amino acid sequence of a peptide can then be deduced from these differences in mass.

MS/MS is particularly useful for detecting and precisely mapping post translational modifications of proteins, such as phosphorylations or acetylations. Because these modifications impart a characteristic mass increase to an amino acid, they are easily detected by mass spectrometry. In combination with rapid purification techniques, mass spectrometry has emerged as a powerful method for detecting posttranslational modifications of proteins and the identity of proteins present in mixtures of proteins.

Two-Dimensional Separation Methods are Especially Powerful

Because different proteins can have similar sizes, shapes, masses, and overall charges, most separation techniques such as SDS polyacrylamide-gel electrophoresis or ion-exchange chromatography cannot typically display all the proteins in a cell or even in an organelle. In contrast, two-dimensional gel electrophoresis, which combines two different separation procedures, can resolve up to 2000 proteins—the total number of different proteins in a simple bacterium—in the form of a two-dimensional protein map.

In the first step, the proteins are separated by their intrinsic charges. The sample is dissolved in a small volume of a solution containing a nonionic (uncharged) detergent, together with β-mercaptoethanol and the denaturing reagent urea. This solution solubilizes, denatures, and dissociates all the polypeptide chains but leaves their intrinsic charge unchanged. The polypeptide chains are then separated in a pH gradient by a procedure called isoelectric focusing, which takes advantage of the variation in the net charge on a protein molecule with the pH of its surrounding solution. Every protein has a characteristic isoelectric point, the pH at which the protein has no net charge and therefore does not migrate in an electric field. In isoelectric focusing, proteins are separated electrophoretically in a narrow tube of polyacrylamide gel in which a gradient of pH is established by a mixture of special buffers. Each protein moves to a position in the gradient that corresponds to its isoelectric point and remains there. This is the first dimension of two-dimensional polyacrylamide-gel electrophoresis.

In the second step, the narrow gel containing the separated proteins is again subjected to electrophoresis but in a direction that is at a right angle to the direction used in the first step. This time SDS is added, and the proteins separate according to their size, as in one-dimensional SDS-PAGE: the original narrow gel is soaked in SDS and then placed on one edge of an SDS polyacrylamide-gel slab, through which each polypeptide chain migrates to form a discrete spot. This is the second dimension of two-dimensional polyacrylamide-gel electrophoresis. The only proteins left unresolved are those that have both identical sizes and identical isoelectric points, a relatively rare situation. Even trace amounts of each polypeptide chain can be detected on the gel by various staining procedures—or by autoradiography if the protein sample was initially labeled with a radioisotope. The technique has such great resolving power that it can distinguish between two proteins that differ in only a single charged amino acid.

A different, even more powerful, “two-dimensional” technique is now available when the aim is to determine all of the proteins present in an organelle or another complex mixture of proteins. Because the technique relies on mass spectroscopy, it requires that the proteins be from an organism with a completely sequenced genome. First, the mixture of proteins present is digested with trypsin to produce short peptides. Next, these peptides are separated by a series of automated liquid chromatography steps. As the second dimension, each separated peptide is fed directly into a tandem mass spectrometer (MS/MS) that allows its amino acid sequence, as well as any post-translational modifications, to be determined. This arrangement, in which a tandem mass spectrometer (MS/MS) is attached to the output of an automated liquid chromatography (LC) system, is referred to as LC-MS/MS. It is now becoming routine to subject an entire organelle preparation to LC-MS/MS analysis and to identify hundreds of proteins and their modifications. Of course, no organelle isolation procedure is perfect, and some of the proteins identified will be contaminating proteins. These can conceivably be excluded by analyzing neighboring fractions from the organelle purification and “subtracting” them out from the peak organelle fractions.

Hydrodynamic Measurements Reveal Size and Shape of a Protein Complex

Most proteins in a cell act as part of larger complexes, and knowledge of the size and shape of these complexes often leads to insights regarding their function. This information can be obtained in several important ways. Sometimes, a complex can be directly visualized using electron microscopy. A complementary approach relies on the hydrodynamic properties of a complex, that is, its behavior as it moves through a liquid medium. Usually, two separate measurements are made. One measure is the velocity of a complex as it moves under the influence of a centrifugal field produced by an ultracentrifuge. The sedimentation constant (or S-value) obtained depends on both the size and the shape of the complex and does not, by itself, convey especially useful information. However, once a second hydrodynamic measurement is performed—by charting the migration of a complex through a gel-filtration chromatography column—both the approximate shape of a complex and its molecular weight can be calculated.

Molecular weight can also be determined more directly by using an analytical ultracentrifuge, a complex device that allows protein absorbance measurements to be made on a sample while it is subjected to centrifugal forces. In this approach, the sample is centrifuged until it reaches equilibrium, where the centrifugal force on a protein complex exactly balances its tendency to diffuse away. Because this balancing point is dependent on a complex's molecular weight but not on its particular shape, the molecular weight can be directly calculated, as needed to determine the stoichiometry of each protein in a protein complex.

Sets of Interacting Proteins can be Identified by Biochemical Methods

Because most proteins in the cell function as part of complexes with other proteins, a preliminary way to begin to characterize the biological role of an unknown protein is to identify all of the other proteins to which it specifically binds.

One method for identifying proteins that bind to one another tightly is coimmunoprecipitation. In this case, an antibody recognizes a specific target protein; reagents that bind to the antibody and are coupled to a solid matrix then drag the complex out of solution to the bottom of a test tube. If the original target protein is associated tightly enough with another protein when it is captured by the antibody, the partner precipitates as well. This method is useful for identifying proteins that are part of a complex inside cells, including those that interact only transiently—for example, when extracellular signal molecules stimulate cells. Another method frequently used to identify a protein's binding partners is protein affinity chromatography. To employ this technique to capture interacting proteins, a target protein is attached to polymer beads that are packed into a column. When the proteins in a cell extract are washed through this column, those proteins that interact with the target protein are retained by the affinity matrix. These proteins can then be eluted and their identity determined by mass spectrometry.

In addition to capturing protein complexes on columns or in test tubes, researchers are developing high-density protein arrays to investigate protein interactions. These arrays, which contain thousands of different proteins or antibodies spotted onto glass slides or immobilized in tiny wells, allow one to examine the biochemical activities and binding profiles of a large number of proteins at once. For example, if one incubates a fluorescently labeled protein with arrays containing thousands of immobilized proteins, the spots that remain fluorescent after extensive washing each contain a protein to which the labeled protein specifically binds.

Protein-Protein Interactions can be Identified by a Two-Hybrid Technique

The yeast two-hybrid system is another way, besides a biochemical approach, to reveal protein-protein interactions. The technique takes advantage of the modular nature of gene activator proteins. These proteins both bind to specific DNA sequences and activate gene transcription, and these activities are often performed by two separate protein domains. Using recombinant DNA techniques, two such protein domains are used to create separate “bait” and “prey” fusion proteins. To create the “bait” fusion protein, the DNA sequence that codes for a target protein is fused with DNA that encodes the DNA-binding domain of a gene activator protein. When this construct is introduced into yeast, the cells produce the fusion protein, with the target protein attached to this DNA-binding domain. This fusion protein binds to the regulatory region of a reporter gene, where it serves as “bait” to fish for proteins that interact with the target protein. To search for potential binding partners (potential prey for the bait), the candidate proteins also have to be constructed as fusion proteins: DNA encoding the activation domain of a gene activator protein is fused to a large number of different genes. Members of this collection of genes—encoding potential “prey” —are introduced individually into yeast cells containing the bait. If the yeast cell receives a DNA clone that expresses a prey partner for the bait protein, the two halves of a transcriptional activator are united, switching on the reporter gene.

This ingenious technique sounds complex, but the two-hybrid system is straightforward to use in the laboratory. Although the protein-protein interactions occur in the yeast cell nucleus, proteins from every part of the cell and from candidate organisms can be studied in this way. The two-hybrid system has been scaled up to map the interactions that occur among many of the proteins an organism produces. In this case, a set of bait and prey fusions is produced for each cell protein, and every bait/prey combination can be monitored. In this way protein interaction maps have been generated for many proteins.

Protein Function can be Selectively Modified with Small Molecules

Chemical inhibitors have contributed to the development of cell biology. Small organic molecules are carbon-based compounds that have molecular weights in the range 100-1000 and contain up to or so carbon atoms. In the past, small molecules were usually natural products. The recent development of methods to synthesize hundreds of thousands of small molecules and to carry out large-scale automated screens holds the promise of identifying chemical antagonists and agonists for virtually any biological process. In such approaches, large collections of small chemical compounds are simultaneously tested, either on living cells or in cell-free assays. Once an antagonist or agonist is identified, it can be used as a probe to identify, through affinity chromatography or other means, the protein to which the antagonist or agonist binds and, if antagonism or agonism of protein function is therapeutic, as a drug in and of itself.

Protein Structure can be Determined Using X-Ray Diffraction

The main technique that has been used to discover the three-dimensional structure of molecules, including proteins, at atomic resolution is x-ray crystallography. X-rays, like light, are a form of electromagnetic radiation, but they have a much shorter wavelength, typically around 0.1 nm (the diameter of a hydrogen atom). If a narrow parallel beam of x-rays is directed at a sample of a pure protein, most of the x-rays pass straight through it. A small fraction, however, are scattered by the atoms in the sample. If the sample is a well-ordered crystal, the scattered waves reinforce one another at certain points and appear as diffraction spots when recorded by a suitable detector.

The position and intensity of each spot in the x-ray diffraction pattern contain information about the locations of the atoms in the crystal that gave rise to it. Deducing the three-dimensional structure of a large molecule from the diffraction pattern of its crystal is a complex task. But in recent years x-ray diffraction analysis has become increasingly automated, and now the slowest step is likely to be the generation of suitable protein crystals. This step requires large amounts of very pure protein and often involves years of trial and error to discover the proper crystallization conditions; the pace has somewhat accelerated with the use of recombinant DNA techniques to produce pure proteins and computerized techniques to test large numbers of crystallization conditions.

Analysis of the resulting diffraction pattern produces a complex three dimensional electron-density map. Interpreting this map—translating its contours into a three-dimensional structure—is a complicated procedure that requires knowledge of the amino acid sequence of the protein. Largely by trial and error, the sequence and the electron-density map are correlated by computer to give the best possible fit. The reliability of the final atomic model depends on the resolution of the original crystallographic data: 0.5 nm resolution might produce a low-resolution map of the polypeptide backbone, whereas a resolution of 0.15 nm allows all of the no hydrogen atoms in the molecule to be reliably positioned.

A complete atomic model is often too complex to appreciate directly, but simplified versions that show a protein's essential structural features can be readily derived from it. The three-dimensional structures of about 20,000 different proteins have now been determined by x-ray crystallography or by NMR spectroscopy—enough to begin to see families of common structures emerging. These structures or protein folds often seem to be more conserved in evolution than are the amino acid sequences that form the a helices and β strands themselves.

NMR can be Used to Determine Protein Structure in Solution

Nuclear magnetic resonance (NMR) spectroscopy has been widely used for many years to analyze the structure of small molecules. This technique is now also increasingly applied to the study of small proteins or protein domains. Unlike x-ray crystallography, NMR does not depend on having a crystalline sample. It simply requires a small volume of concentrated protein solution that is placed in a strong magnetic field; indeed, it is the main technique that yields detailed evidence about the three-dimensional structure of molecules in solution.

Certain atomic nuclei, particularly hydrogen nuclei, have a magnetic moment or spin: that is, they have an intrinsic magnetization, like a bar magnet. The spin aligns along the strong magnetic field, but it can be changed to a misaligned, excited state in response to applied radiofrequency (RF) pulses of electromagnetic radiation. When the excited hydrogen nuclei return to their aligned state, they emit RF radiation, which can be measured and displayed as a spectrum. The nature of the emitted radiation depends on the environment of each hydrogen nucleus, and if one nucleus is excited, it influences the absorption and emission of radiation by other nuclei that lie close to it. It is consequently possible, by an ingenious elaboration of the basic NMR technique known as two-dimensional NMR, to distinguish the signals from hydrogen nuclei in different amino acid residues, and to identify and measure the small shifts in these signals that occur when these hydrogen nuclei lie close enough together to interact. Because the size of such a shift reveals the distance between the interacting pair of hydrogen atoms, NMR can provide information about the distances between the parts of the protein molecule. By combining this information with a knowledge of the amino acid sequence, it is possible in principle to compute the three-dimensional structure of the protein.

For technical reasons the structure of small proteins of about 20,000 daltons or less can be most readily determined by NMR spectroscopy. Resolution decreases as the size of a macromolecule increases. But recent technical advances have now pushed the limit to about 100,000 daltons, thereby making the majority of proteins accessible for structural analysis by NMR.

Protein Sequence and Structure Provide Clues about Protein Function

Having discussed methods for purifying and analyzing proteins, we now turn to a common situation in cell and molecular biology: an investigator has identified a gene important for a biological process but has no direct knowledge of the biochemical properties of its protein product.

Thanks to the proliferation of protein and nucleic acid sequences that are catalogued in genome databases, the function of a gene—and its encoded protein—can conceivably be predicted by simply comparing its sequence with those of previously characterized genes. Because amino acid sequence determines protein structure, and structure dictates biochemical function, proteins that share a similar amino acid sequence usually have the same structure and usually perform similar biochemical functions, even when they are found in distantly related organisms. In modern cell biology, the study of a newly discovered protein usually begins with a search for previously characterized proteins that are similar in their amino acid sequences.

Searching a collection of known sequences for similar genes or proteins is typically done over the World Wide Web, and it conventionally involves selecting a database and entering the desired sequence. A sequence alignment program—the most popular are BLAST and FASTA—scans the database for similar sequences by sliding the submitted sequence along the archived sequences until a cluster of residues falls into full or partial alignment. The results of even a complex search—which can be performed on either a nucleotide or an amino acid sequence—are returned within a short time. Such comparisons can predict the functions of individual proteins, families of proteins, or even much of the protein complement of a newly sequenced organism.

Many proteins that adopt the same conformation and have related functions are too distantly related to be identified as clearly similar from a comparison of their amino acid sequences alone. Thus, an ability to reliably predict the three dimensional structure of a protein from its amino acid sequence would improve our ability to infer protein function from the sequence information in genomic databases. In recent years, major progress has been made in predicting the precise structure of a protein. These predictions are based, in part, on our knowledge of tens of thousands of protein structures that have already been determined by x-ray crystallography and NMR spectroscopy and, in part, on computations using our knowledge of the physical forces acting on the atoms. The goal is to predict the structures of proteins that are large or have multiple domains, or predict structures at the very high levels of resolution needed to assist in computer-based drug discovery.

Sequence databases can be searched (or two or more sequences can be aligned) to find similar amino acid or nucleic acid sequences. For example, a BLAST search for proteins similar to the human cell-cycle regulatory protein Cdc2 (Query) locates maize Cdc2 (Sbjct), which is 68% identical (and 82% similar) to human Cdc2 in its amino acid sequence. The alignment begins at residue 57 of the Query protein, suggesting that the human protein has an N-terminal region that is absent from the maize protein. The results of the BLAST search indicate differences in sequence as well as similarities, and when the two amino acid sequences are identical as well as when conservative amino acids are substituted. Here, only one small gap needs to be introduced, at position 194 in the Query sequence, to align the two sequences maximally. The alignment score (Score), which is expressed in two different types of units, takes into account penalties for substitutions and gaps; the higher the alignment score, the better the match. The significance of the alignment is reflected in the Expectation (E) value, which specifies how often a match this good would be expected to occur by chance. The lower the E value, the more significant the match; the very low value in this instance e-111 indicates certain significance. E values much higher than 0.1 are unlikely to reflect true relatedness. For example, an E value of 0.1 means there is a 1 in 10 likelihood that such a match would arise solely by chance.

Protein sequence alignments use standard substitution matrices, for example, the BLOSUM62 matrix, that take into account matches and mismatches of different types (such as a proline to valine, or isoleucine to leucine) based on their different physicochemical and evolutionary properties. Amino acids that are physicochemically similar to one another are determined by their side chains. The common amino acids are grouped according to whether their side chains are acidic, basic, uncharged polar, or nonpolar. Of the amino acids found in proteins, there are equal numbers of polar and non-polar side chains. However, some side chains considered polar are large enough to have some non-polar properties, e.g., Tyr, Thr, Arg, and Lys. Here is the list of amino acids, with their 3 letter abbreviation, 1 letter abbreviation, and grouping by side chain.

| Amino acid | 3 letter name | 1 letter name | Side chain |
|---|---|---|---|
| Aspartic acid | Asp | D | Negative (polar) |
| Glutamic acid | Glu | E | Negative (polar) |
| Arginine | Arg | R | Positive (polar) |
| Lysine | Lys | K | Positive (polar) |
| Histidine | His | H | Positive (polar) |
| Asparagine | Asn | N | Uncharged polar |
| Glutamine | Gln | Q | Uncharged polar |
| Serine | Ser | S | Uncharged polar |
| Threonine | Thr | T | Uncharged polar |
| Tyrosine | Tyr | Y | Uncharged polar |
| Alanine | Ala | A | Nonpolar |
| Glycine | Gly | G | Nonpolar |
| Valine | Val | V | Nonpolar |
| Leucine | Leu | L | Nonpolar |
| Isoleucine | Ile | I | Nonpolar |
| Proline | Pro | P | Nonpolar |
| Phenylalanine | Phe | F | Nonpolar |
| Methionine | Met | M | Nonpolar |
| Tryptophan | Trp | W | Nonpolar |
| Cysteine | Cys | C | Nonpolar |

Generally speaking, one requires a 30% identity in sequence to consider that two polypeptides match. While finding similar sequences and structures for a new protein will provide many clues about its function, it may be necessary to test these insights through direct experimentation. However, the clues generated from sequence comparisons traditionally point the investigator in the correct experimental direction. The use of sequence alignments has therefore become one of the choicest strategies in modern cell biology.

Analyzing and Manipulating DNA

Technical breakthroughs in genetic engineering—the ability to manipulate DNA with precision in a test tube or an organism—have had a dramatic impact on all aspects of cell biology by facilitating the study of cells and their macromolecules in previously unimagined ways. Recombinant DNA technology comprises a mixture of techniques, some newly developed and some borrowed from other fields. Central to the technology are the following key techniques: 1. Cleavage of DNA at specific sites by restriction nucleases, which greatly facilitates the isolation and manipulation of individual genes. 2. DNA ligation, which makes it possible to design and construct DNA molecules that are not found in nature. 3. DNA cloning through the use of either cloning vectors or the polymerase chain reaction, in which a portion of DNA is repeatedly copied to generate many billions of identical molecules. 4. Nucleic acid hybridization, which makes it possible to find a specific sequence of DNA or RNA with great accuracy and sensitivity on the basis of its ability to selectively bind a complementary nucleic acid sequence. 5. Determination of the sequence of nucleotides of any DNA (even entire genomes), making it possible to identify genes and to deduce the amino acid sequence of the proteins they encode. 6. Simultaneous monitoring of the level of mRNA produced by genes in a cell using nucleic acid microarrays, in which tens of thousands of hybridization reactions take place simultaneously.

Restriction Nucleases Cut Large DNA Molecules into Fragments

Unlike a protein, a gene does not exist as a discrete entity in cells, but rather as a small region of a much longer DNA molecule. Although the DNA molecules in a cell can be randomly broken into small pieces by mechanical force, a fragment containing a single gene in a mammalian genome would still be only one among a hundred thousand or more DNA fragments, indistinguishable in their average size. How could such a gene be purified? Because all DNA molecules consist of an approximately equal mixture of the same four nucleotides, they cannot be readily separated, as proteins can, on the basis of their different charges and binding properties.

The solution to all of these problems began to emerge with the discovery of restriction nucleases. These enzymes, which can be purified from bacteria, cut the DNA double helix at specific sites defined by the local nucleotide sequence, thereby cleaving a long double-stranded DNA molecule into fragments of strictly defined sizes. Different restriction nucleases have different sequence specificities, and it is straightforward to find an enzyme that can create a DNA fragment that includes a particular gene. The size of the DNA fragment can then be used as a basis for partial purification of the gene from a mixture.

Different species of bacteria make different restriction nucleases, which protect them from viruses by degrading incoming viral DNA. Each bacterial nuclease recognizes a specific sequence of four to eight nucleotides in DNA. These sequences, where they occur in the genome of the bacterium itself, are protected from cleavage by methylation at an A or a C nucleotide; the sequences in foreign DNA are generally not methylated and so are cleaved by the restriction nucleases. Large numbers restriction nucleases have been purified from various species of bacteria; several hundred, most of which recognize different nucleotide sequences, are now available commercially.

Some restriction nucleases produce staggered cuts, which leave short single stranded tails at the two ends of each fragment. Ends of this type are known as cohesive ends, as each tail can form complementary base pairs with the tail at any other end produced by the same enzyme. The cohesive ends generated by restriction enzymes allow any two DNA fragments to be easily joined together, as long as the fragments were generated with the same restriction nuclease (or with another nuclease that produces the same cohesive ends). DNA molecules produced by splicing together two or more DNA fragments are called recombinant DNA molecules.

Gel Electrophoresis Separates DNA Molecules of Different Sizes

The same types of gel electrophoresis methods that have proved so useful in the analysis of proteins can determine the length and purity of DNA molecules. The procedure is actually simpler than for proteins: because each nucleotide in a nucleic acid molecule already carries a single negative charge (on the phosphate group), there is no need to add the negatively charged detergent SDS that is required to make protein molecules move uniformly toward the positive electrode. For DNA fragments less than 500 nucleotides long, specially designed polyacrylamide gels allow the separation of molecules that differ in length by as little as a single nucleotide. The pores in polyacrylamide gels, however, are too small to permit very large DNA molecules to pass; to separate these by size, the much more porous gels formed by dilute solutions of agarose (a polysaccharide isolated from seaweed) are used. These DNA separation methods are widely used for both analytical and preparative purposes.

A variation of agarose-gel electrophoresis, called pulsed-field gel electrophoresis, makes it possible to separate even extremely long DNA molecules. Ordinary gel electrophoresis fails to separate such molecules because the steady electric field stretches them out so that they travel end-first through the gel in snakelike configurations at a rate that is independent of their length. In pulsed-field gel electrophoresis, by contrast, the direction of the electric field changes periodically, which forces the molecules to reorient before continuing to move snakelike through the gel. This reorientation takes much more time for larger molecules, so that longer molecules move more slowly than shorter ones. As a consequence, even entire bacterial or yeast chromosomes separate into discrete bands in pulsed-field gels and so can be sorted and identified on the basis of their size. Although a typical mammalian chromosome of 10̂8 base pairs is too large to be sorted even in this way, large segments of these chromosomes are readily separated and identified if the chromosomal DNA is first cut with a restriction nuclease selected to recognize sequences that occur only rarely (once every 10,000 or more nucleotide pairs).

The DNA bands on agarose or polyacrylamide gels are invisible unless the DNA is labeled or stained in some way. One sensitive method of staining DNA is to expose it to the dye ethidium bromide, which fluoresces under ultraviolet light when it is bound to DNA. An even more sensitive detection method incorporates a radioisotope into the DNA molecules before electrophoresis; 32P is often used as it can be incorporated into DNA phosphates and emits an energetic β particle that is easily detected by autoradiography.

DNA can be Labeled with Radioisotopes or Chemical Markers In Vitro

Two procedures are widely used to label isolated DNA molecules. In the first method, a DNA polymerase copies the DNA in the presence of nucleotides that are either radioactive (usually labeled with 32P) or chemically tagged. In this way, “DNA probes” containing many labeled nucleotides can be produced for nucleic acid hybridization reactions. The second procedure uses the bacteriophage enzyme polynucleotide kinase to transfer a single 32P-labeled phosphate from ATP to the 5′ end of each DNA chain. Because only one 32P atom is incorporated by the kinase into each DNA strand, the DNA molecules labeled in this way are often not radioactive enough to be used as DNA probes; because they are labeled at only one end, however, they have been invaluable for other applications, including DNA footprinting.

Radioactive labeling methods are being replaced by labeling with molecules that can be detected chemically or through fluorescence. To produce such nonradioactive DNA molecules, specially modified nucleotide precursors are used. A DNA molecule made in this way is allowed to bind to its complementary DNA sequence by hybridization, and is then detected with an antibody (or other ligand) that specifically recognizes its modified side chain.

Nucleic Acid Hybridization Detects Specific Nucleotide Sequences

When an aqueous solution of DNA is heated at 100° C. or exposed to a very high pH (pH>13), the complementary base pairs that normally hold the two strands of the double helix together are disrupted and the double helix rapidly dissociates into two single strands. This process, called DNA denaturation, was for many years thought to be irreversible. It was discovered, however, that complementary single strands of DNA readily re-form double helices by a process called hybridization (also called DNA renaturation) if they are kept for a prolonged period at 65° C. Similar hybridization reactions can occur between any two single-stranded nucleic acid chains (DNA/DNA, RNA/RNA, or RNA/DNA), provided that they have complementary nucleotide sequences. These specific hybridization reactions are widely used to detect and characterize specific nucleotide sequences in both RNA and DNA molecules.

Single-stranded DNA molecules used to detect complementary sequences are known as probes; these molecules, which carry radioactive or chemical markers to facilitate their detection, can range from fifteen to thousands of nucleotides long. Hybridization reactions using DNA probes are so sensitive and selective that they can detect complementary sequences present at a concentration as low as one molecule per cell. It is thus possible to determine how many copies of any DNA sequence are present in a particular DNA sample. The same technique can be used to search for similar but nonidentical genes. To find a gene of interest in an organism whose genome has not yet been sequenced, for example, a portion of a known gene can be used as a probe.

Stringent versus nonstringent hybridization conditions tell sequences apart. To use a DNA probe to find an almost identical match, high stringent hybridization conditions are used; the reaction temperature is kept just a few degrees below that at which a perfect DNA helix denatures in the solvent used (its melting temperature), so that all imperfect helices formed are unstable. Lowering the salt concentration lowers the melting point, as does the addition of formamide. As an example, hybridization is in 50% formamide at 42° C. When a DNA probe is being used to find DNAs with similar, as well as identical, sequences, low stringent conditions are used; hybridization is performed at a lower temperature, which allows even imperfectly paired double helices to form. Continuing with this example, hybridization is in 50% formamide at 35° C. The lower temperature hybridization conditions are used to search for genes that are nonidentical but similar.

Alternatively, DNA probes can be used in hybridization reactions with RNA rather than DNA to find out whether a cell is expressing a given gene. In this case a DNA probe that contains part of the gene's sequence is hybridized with RNA purified from the cell in question to see whether the RNA includes nucleotide sequences matching the probe DNA and, if so, in what quantities. In somewhat more elaborate procedures, the DNA probe is treated with specific nucleases after the hybridization is complete, to determine the exact regions of the DNA probe that have paired with the RNA molecules. One can thereby determine the start and stop sites for RNA transcription, as well as the precise boundaries of the intron and exon sequences in a gene.

Today, the positions of intron/exon boundaries are usually determined by sequencing the complementary DNA (cDNA) sequences that represent the mRNAs expressed in a cell and comparing them with the nucleotide sequence of the genome. We describe later how cDNAs are prepared from mRNAs.

The hybridization of DNA probes to RNAs allows one to determine whether or not a particular gene is being transcribed; moreover, when the expression of a gene changes, one can determine whether the change is due to transcriptional or posttranscriptional controls. These tests of gene expression were initially performed with one DNA probe at a time. DNA microarrays now allow the simultaneous monitoring of hundreds or thousands of genes at a time. Hybridization methods are still in wide use in cell biology today.

Blotting Facilitates Hybridization with Separated Nucleic Acid Molecules

Specific RNA or DNA molecules are detected by gel-transfer hybridization in a method called Southern blotting (named after its inventor) or Northern blotting (named with reference to Southern blotting). To start, one collects tissue from a source and disrupts the cells in a strong detergent to inactivate nucleases that might otherwise degrade the nucleic acids. Next, one separates the RNA and DNA from all of the other cell components: the proteins present are completely denatured and removed by repeated extractions with phenol—a potent organic solvent that is partly miscible with water; the nucleic acids, which remain in the aqueous phase, are then precipitated with alcohol to separate them from the small molecules of the cell. Then one separates the DNA from the RNA by their different solubilities in alcohols and degrades any contaminating nucleic acid of the unwanted type by treatment with a highly specific enzyme—either an RNase or a DNase. The mRNAs are typically separated from bulk RNA by retention on a chromatography column that specifically binds the poly-A tails of mRNAs.

In this example, the DNA probe is detected by its radioactivity. DNA probes detected by chemical or fluorescence methods are also widely used. First, a mixture of either single-stranded RNA molecules (Northern blotting) or the double-stranded DNA fragments created by restriction nuclease treatment (Southern blotting) is separated according to length by electrophoresis. Next, a sheet of nitrocellulose or nylon paper is laid over the gel, and the separated RNA or DNA fragments are transferred to the sheet by blotting. Then, the nitrocellulose sheet is carefully peeled off the gel. Next, the sheet containing the bound nucleic acids is placed in a sealed plastic bag together with a buffered salt solution containing a radioactively labeled DNA probe. The sheet is exposed to a labeled DNA probe for a prolonged period under conditions favoring hybridization. Last, the sheet is removed from the bag and washed thoroughly, so that only probe molecules that have hybridized to the RNA or DNA immobilized on the paper remain attached. After autoradiography, the DNA that has hybridized to the labeled probe shows up as bands on the autoradiograph. For Southern blotting, the strands of the double-stranded DNA molecules on the paper must be separated before the hybridization process; this is done by exposing the DNA to alkaline denaturing conditions after the gel has been run.

Genes can be Cloned Using DNA Libraries

Genes can be cloned using DNA libraries. Almost any DNA fragment can be cloned. In molecular biology, the term DNA cloning is used in two senses. In one sense, it literally refers to the act of making many identical copies of a DNA molecule—the amplification of a particular DNA sequence. However, the term also describes the isolation of a particular stretch of DNA (often a particular gene) from the rest of a cell's DNA, because this isolation is greatly facilitated by making many identical copies of the DNA of interest. In both cases, cloning refers to the act of making many genetically identical copies.

DNA cloning in its most general sense can be accomplished in several ways. The simplest involves inserting a particular fragment of DNA into the purified DNA genome of a self-replicating genetic element—generally a virus or a plasmid. A DNA fragment containing a human gene, for example, can be joined in a test tube to the chromosome of a bacterial virus, and the new recombinant DNA molecule can then be introduced into a bacterial cell, where the inserted DNA fragment will be replicated along with the DNA of the virus. Starting with only one such recombinant DNA molecule that infects a single cell, the normal replication mechanisms of the virus can produce more than 10 to the power of 12 identical virus DNA molecules in a single day, thereby amplifying the amount of the inserted human DNA fragment by the same factor. A virus or plasmid used in this way is known as a cloning vector, and the DNA propagated by insertion into it is said to have been cloned.

To isolate a specific gene, one begins by constructing a DNA library—a comprehensive collection of cloned DNA fragments from a cell, tissue, or organism. This library includes (one hopes) at least one fragment that contains the gene of interest. Libraries can be constructed with either a virus or a plasmid vector and are generally housed in a population of bacterial cells. The principles underlying the methods used for cloning genes are the same for either type of cloning vector, although the details may differ. Today, most cloning is performed with plasmid vectors.

The plasmid vectors most widely used for gene cloning are small circular molecules of double-stranded DNA derived from larger plasmids that occur naturally in bacterial cells. They generally account for only a minor fraction of the total host bacterial cell DNA, but they can easily be separated owing to their small size from chromosomal DNA molecules, which are large and precipitate as a pellet upon centrifugation. For use as cloning vectors, the purified plasmid DNA circles are first cut with a restriction nuclease to create linear DNA molecules. The genomic DNA to be used in constructing the library is cut with the same restriction nuclease, and the resulting restriction fragments (including those containing the gene to be cloned) are then added to the cut plasmids and annealed via their cohesive ends to form recombinant DNA circles. These recombinant molecules containing foreign DNA inserts are then covalently sealed with the enzyme DNA ligase.

In the next step in preparing the library, the recombinant DNA circles are introduced into bacterial cells that have been made transiently permeable to DNA. These bacterial cells are now said to be transfected with the plasmids. As the cells grow and divide, doubling in number every minutes, the recombinant plasmids also replicate to produce an enormous number of copies of DNA circles containing the foreign DNA. Many bacterial plasmids carry genes for antibiotic resistance, a property that can be exploited to select those cells that have been successfully transfected; if the bacteria are grown in the presence of the antibiotic, only cells containing plasmids will survive. Each original bacterial cell that was initially transfected contains, in general, a different foreign DNA insert; this insert is inherited by all of the progeny cells of that bacterium, which together form a small colony in a culture dish.

For many years, plasmids were used to clone fragments of DNA of 1000 to 30,000 nucleotide pairs. Larger DNA fragments are more difficult to handle and were harder to clone. Today, new plasmid vectors based on the naturally occurring F plasmid of E. coli are used to clone DNA fragments of 300,000 to 1 million nucleotide pairs. Unlike smaller bacterial plasmids, the F plasmid—and its derivative, the bacterial artificial chromosome (BAC)—is present in only one or two copies per E. coli cell. The fact that BACs are kept in such low numbers in bacterial cells may contribute to their ability to maintain large cloned DNA sequences stably: with only a few BACs present, it is less likely that the cloned DNA fragments will become scrambled by recombination with sequences carried on other copies of the plasmid. Because of their stability, ability to accept large DNA inserts, and ease of handling, BACs are now the preferred vector for building DNA libraries of complex organisms—including those representing the human genome.

Two Types of DNA Libraries Serve Different Purposes

Cleaving the entire genome of a cell with a specific restriction nuclease and cloning each fragment as just described produces a very large number of DNA fragments—on the order of a million for a mammalian genome. The fragments are distributed among millions of different colonies of transfected bacterial cells. Each of the colonies is composed of a clone of cells derived from a single ancestor cell, and therefore harbors many copies of a particular stretch of the fragmented genome. Such a plasmid is said to contain a genomic DNA clone, and the entire collection of plasmids is called a genomic DNA library. But because the genomic DNA is cut into fragments at random, only some fragments contain genes. Many of the genomic DNA clones obtained from the DNA of a higher eukaryotic cell contain only noncoding DNA, which makes up most of the DNA in such genomes.

An alternative strategy is to begin the cloning process by selecting only those DNA sequences that are transcribed into mRNA and thus are presumed to correspond to protein-encoding genes. This is done by extracting the mRNA from cells and then making a DNA copy of each mRNA molecule present—a so-called complementary DNA, or cDNA. The copying reaction is catalyzed by the reverse transcriptase enzyme of retroviruses, which synthesizes a complementary DNA chain on an RNA template. The single-stranded cDNA molecules synthesized by the reverse transcriptase are converted into double-stranded cDNA molecules by DNA polymerase, and these molecules are inserted into a plasmid or virus vector and cloned. Each clone obtained in this way is called a cDNA clone, and the entire collection of clones derived from one mRNA preparation constitutes a cDNA library.

cDNA Clones Contain Uninterrupted Coding Sequences

There are some important differences between genomic DNA clones and cDNA clones. Genomic clones represent a random sample of all the DNA sequences in an organism and, with very rare exceptions, are the same regardless of the cell type used to prepare them. By contrast, cDNA clones contain only those regions of the genome that have been transcribed into mRNA. Because the cells of different tissue types produce distinct sets of mRNA molecules, a distinct cDNA library is obtained for each type of cell used to prepare the library.

The most important advantage of cDNA clones is that they contain the uninterrupted coding sequence of a gene. Eukaryotic genes usually consist of short coding sequences of DNA (exons) separated by much longer noncoding sequences (introns); the production of mRNA entails the removal of the noncoding sequences from the initial RNA transcript and the splicing together of the coding sequences. Bacterial cells will not make these modifications to the RNA produced from a gene of a higher eukaryotic cell. Thus, when the aim of the cloning is either to deduce the amino acid sequence of the protein from the DNA sequence or to produce the protein in bulk by expressing the cloned gene in a bacterial cell, it is much preferable to start with cDNA. cDNA libraries have the additional advantage of representing alternatively spliced mRNAs produced from a given cell or tissue.

Genomic and cDNA libraries are widely shared among investigators and, today, many such libraries are also available from commercial sources.

Genes can be Selectively Amplified by PCR

Now that so many genome sequences are available, genes can be cloned directly without the need to first construct DNA libraries. A technique called polymerase chain reaction (PCR) makes this rapid cloning possible. Starting with an entire genome, PCR allows the DNA from a selected region to be amplified several billionfold, effectively “purifying” this DNA away from the remainder of the genome.

To begin, a pair of DNA oligonucleotides, chosen to flank the desired nucleotide sequence of the gene, are synthesized by chemical methods. These oligonucleotides are then used to prime DNA synthesis on single strands generated by heating the DNA from the entire genome. The newly synthesized DNA is produced in a reaction catalyzed in vitro by a purified DNA polymerase, and the primers remain at the 5′ ends of the final DNA fragments that are made.

Nothing special is produced in the first cycle of DNA synthesis; the power of the PCR method is revealed only after repeated rounds of DNA synthesis. Every cycle doubles the amount of DNA synthesized in the previous cycle. Because each cycle requires a brief heat treatment to separate the two strands of the template DNA double helix, the technique requires the use of a special DNA polymerase, isolated from a thermophilic bacterium, that is stable at much higher temperatures than normal so that it is not denatured by the repeated heat treatments. With each round of DNA synthesis, the newly generated fragments serve as templates in their turn, and within a few cycles the predominant product is a single species of DNA fragment whose length corresponds to the distance between the two original primers.

In practice, effective DNA amplification requires 20-30 reaction cycles, with the products of each cycle serving as the DNA templates for the next—hence the term polymerase “chain reaction.” A single cycle requires only about 5 minutes, and the entire procedure can be easily automated. PCR thereby makes possible the “cell-free molecular cloning” of a DNA fragment in a few hours, compared with the several days for standard cloning procedures. This technique is now used routinely to clone DNA from genes of interest directly—starting either from genomic 5 DNA or from mRNA isolated from cells.

The PCR method is extremely sensitive; it can detect a single DNA molecule in a sample. Trace amounts of RNA can be analyzed in the same way by first transcribing them into DNA with reverse transcriptase. The PCR cloning technique has largely replaced Southern blotting for the diagnosis of genetic diseases and for the detection of low levels of viral infection.

Expression of Genes can be Measured Using Quantitative RT-PCR

It is often desirable to quantitate gene expression by directly measuring mRNA levels in cells. Although Northern blots can be adapted to this purpose, a more accurate method is based on the principles of PCR. This method, called quantitative RT-PCR (reverse transcription-polymerase chain reaction), begins with the total population of mRNA molecules purified from a tissue or a cell culture. It is important that no DNA be present in the preparation; it must be purified away or enzymatically degraded. Two DNA primers that specifically match the gene of interest are added, along with reverse transcriptase, DNA polymerase, and the four deoxynucleoside triphosphates needed for DNA synthesis. The first round of synthesis is the reverse transcription of the mRNA into DNA using one of the primers. Next, a series of heating and cooling cycles allows the amplification of that DNA strand by conventional PCR. The quantitative part of this method relies on a direct relationship between the rate at which the PCR product is generated and the original concentration of the mRNA species of interest. By adding chemical dyes to the PCR reaction that fluoresce only when bound to double-stranded DNA, a simple fluorescence measurement can be used to track the progress of the reaction and thereby accurately deduce the starting concentration of the mRNA that is amplified. Although it seems complicated, this quantitative RT-PCR technique (sometimes called real time PCR) is straightforward to perform in the laboratory; it has displaced Northern blotting as the method of choice for quantifying mRNA levels from any given gene.

Cells can be Used as Factories to Produce Specific Proteins

The vast majority of the thousands of different proteins in a cell, including many with crucially important functions, are present in very small amounts. In the past, for most of them, it has been extremely difficult, if not impossible, to obtain more than a few micrograms of pure material. One of the most important contributions of DNA cloning and genetic engineering to cell biology is that they have made it possible to produce almost any of the cell's proteins in a nearly unlimited amount.

Large amounts of the desired protein are produced in living cells by using expression vectors. These are generally plasmids that have been designed to produce a large amount of a stable mRNA that can be efficiently translated into protein in the transfected bacterial, yeast, insect, or mammalian cell. A plasmid vector is engineered to contain a highly active promoter, which causes unusually large amounts of mRNA to be produced from an adjacent protein-coding gene inserted into the plasmid vector. Depending on the characteristics of the cloning vector, the plasmid is introduced into bacterial, yeast, insect, or mammalian cells, where the inserted gene is efficiently transcribed and translated into protein.

Because the desired protein made from an expression vector is produced inside a cell, it must be purified away from the host-cell proteins by chromatography after cell lysis; but because it is a plentiful species in the cell lysate (often 1-10% of the total cell protein), the purification is usually easy to accomplish in only a few steps. In order to purify a protein, it first must be extracted from inside the cell, unless it is secreted into the medium. The cells are typically homogenized to produce a homogenate or slurry. The homogenate is typically fractionated into different components by centrifugation. After centrifugation, proteins are often separated by chromatography. Secreted, soluble proteins are isolated from the supernatants of infected cells after pelleting the cells by centrifugation and do not require cell lysis. A variety of expression vectors are available, each engineered to function in the type of cell in which the protein is to be made. In this way, cells can be induced to make vast quantities of proteins useful for medical purposes or to be studied for structure and function.

Genes can be Engineered by Site-Directed Mutagenesis

A technique called site-directed mutagenesis changes selected amino acids in a protein. To begin, a recombinant plasmid containing a gene insert is separated into its two DNA strands. A synthetic oligonucleotide primer corresponding to part of the gene sequence but containing a single altered nucleotide at a predetermined point is added to the single-stranded DNA under conditions that permit imperfect DNA hybridization. The primer hybridizes to the DNA, forming a single mismatched nucleotide pair. The recombinant plasmid is made double-stranded by in vitro DNA synthesis (starting from the primer) followed by sealing by DNA ligase. The double10 stranded DNA is introduced into a cell, where it is replicated. Replication using one strand of the template produces a normal DNA molecule, but replication using the other strand (the one that contains the primer) produces a DNA molecule carrying the desired mutation. Only half of the progeny cells will end up with a plasmid that contains the desired mutant gene. However, a progeny cell that contains the mutated gene can be identified, separated from other cells, and cultured to produce a pure population of cells, all of which carry the mutated gene. With an oligonucleotide of the appropriate sequence, more than one amino acid substitution can be made at a time, or one or more amino acids can be inserted or deleted. It is also possible to create a site-directed mutation by using the appropriate oligonucleotides and PCR (instead of plasmid replication) to amplify the mutated gene.

Proteins and Nucleic Acids can be Synthesized by Chemical Reactions

Chemical reactions have been devised to synthesize directly specific sequences of amino acids or nucleic acids. These methodologies provide direct sources of biological molecules and do not rely on any cells or enzymes. Chemical synthesis is the method of choice for obtaining nucleic acids in the range of 100 nucleotides or fewer, which, under the basic concept of de novo gene synthesis, may be assembled into larger constructs using some form of polymerase chain assembly or ligase chain reaction approach. Chemical synthesis is also routinely used to produce specific peptides that, when chemically coupled to other proteins, are used to generate antibodies against the peptide.

DNA can be Rapidly Sequenced

The dideoxy method for sequencing DNA is based on in vitro DNA synthesis performed in the presence of chain-terminating dideoxyribonucleoside triphosphates. This method relies on the use of dideoxyribonucleoside triphosphates, derivatives of the normal deoxyribonucleoside triphosphates that lack the 3′ hydroxyl group. Purified DNA is synthesized in vitro in a mixture that contains single-stranded molecules of the DNA to be sequenced, the enzyme DNA polymerase, a short primer DNA to enable the polymerase to start DNA synthesis, and the four deoxyribonucleoside triphosphates (dATP, dCTP, dGTP, dTTP). If a dideoxyribonucleotide analog of one of these nucleotides is also present in the nucleotide mixture, it can become incorporated into a growing DNA chain. Because this chain now lacks a 3′ OH group, the addition of the next nucleotide is blocked, and the DNA chain terminates at that point. As an example, if a small amount of dideoxy ATP (ddATP) is added to the nucleotide mixture, it competes with an excess of the normal deoxyATP (dATP), so that ddATP is occasionally incorporated, at random, into a growing DNA strand. This reaction mixture will eventually produce a set of DNAs of different lengths complementary to the template DNA that is being sequenced and terminating at each of the different As. The exact lengths of the DNA synthesis products can then be used to determine the position of each A in the growing chain. To determine the complete sequence of a DNA fragment, the doublestranded DNA is first separated into its single strands and one of the strands is used as the template for sequencing. Four different chain-terminating dideoxyribonucleoside triphosphates (ddATP, ddCTP, ddGTP, ddTTP) are used in four separate DNA synthesis reactions on copies of the same single-stranded DNA template. Each reaction produces a set of DNA copies that terminate at different points in the sequence. The products of these four reactions are separated by electrophoresis in four parallel lanes of a polyacrylamide gel. The newly synthesized fragments are detected by a label (either radioactive or fluorescent) that has been incorporated either into the primer or into one of the deoxyribonucleoside triphosphates used to extend the DNA chain. In each lane, the bands represent fragments that have terminated at a given nucleotide but at different positions in the DNA. By reading off the bands in order, starting at the bottom of the gel and working across all lanes, the DNA sequence of the newly synthesized strand can be determined. This sequence is complementary to the template strand from the original double-stranded DNA molecule, and identical to a portion of the 5′-to-3′ strand.

Today, sequencing is often carried out by automated machines that use fluorescent dyes and laser scanners. The dideoxy reaction is also used here, but the ddNTPs used in the reaction are labeled with a fluorescent dye, and a different colored dye is used for each type of dideoxynucleotide. In this case, the four sequencing reactions can take place in the same test tube and can be placed in the same well during electrophoresis. The most recently developed sequencing machines carry out electrophoresis in gel-containing capillary tubes. During electrophoresis, the fragments migrate through the gel according to size, and the fluorescent dye on the DNA is activated by a laser beam and detected by an optical scanner. The results are printed as a set of peaks on a graph.

Nucleotide Sequences Predict the Amino Acid Sequences of Proteins.

Now that DNA sequencing is so rapid and reliable, it has become the preferred method for determining, indirectly, the amino acid sequences of most proteins. Given a nucleotide sequence that encodes a protein, the procedure is quite straightforward. Although in principle there are six different reading frames in which a DNA sequence can be translated into protein (three on each strand), the correct one is generally recognizable as the only one lacking frequent stop codons. A random sequence of nucleotides, read in frame, will encode a stop signal for protein synthesis about once every 20 amino acids. Nucleotide sequences that encode a stretch of amino acids much longer than this are candidates for presumptive exons, and they can be translated (by computer) into amino acid sequences and checked against databases for similarities to known proteins from other organisms. If necessary, a limited amount of amino acid sequence can then be determined from the purified protein to confirm the sequence predicted from the DNA.

The problem comes, however, in determining which nucleotide sequences—within a whole genome—represent genes that encode proteins. Identifying genes is easiest when the DNA sequence is from a bacterial or archaeal chromosome, which lacks introns, or from a cDNA clone. The location of genes in these nucleotide sequences can be predicted by examining the DNA for certain distinctive features. Briefly, these genes that encode proteins are identified by searching the nucleotide sequence for open reading frames (ORFs) that begin with an initiation codon, usually ATG, and end with a termination codon, TAA, TAG, or TGA. To minimize errors, computers used to search for ORFs are often directed to count as genes only those sequences that are longer than, say, 100 codons in length.

For more complex genomes, such as those of animals and plants, the presence of large introns embedded within the coding portion of genes complicates the process. In many multicellular organisms, including humans, the average exon is only 150 nucleotides long. Thus one must also search for other features that signal the presence of a gene, for example, sequences that signal an intron/exon boundary or distinctive upstream regulatory regions. Recent efforts to solve the exon prediction problem have turned to artificial intelligence algorithms, in which the computer learns, based on known examples, what sets of features are most indicative of an exon boundary.

A second major approach to identifying the coding regions in chromosomes is through the characterization of the nucleotide sequences of the detectable mRNAs (using the corresponding cDNAs). The mRNAs (and the cDNAs produced from them) lack introns, regulatory DNA sequences, and the nonessential “spacer” DNA that lies between genes. It is therefore useful to sequence large numbers of cDNAs to produce a very large database of the coding sequences of an organism. These sequences are then readily used to distinguish the exons from the introns in the long chromosomal DNA sequences that correspond to genes.

The Genomes of Many Organisms have been Fully Sequenced

Owing in large part to the automation of DNA sequencing, the genomes of many organisms have been fully sequenced; these include plant chloroplasts and animal mitochondria, large numbers of bacteria, and archaea, and many of the model organisms that are studied routinely in the laboratory, including many yeasts, a nematode worm, the fruit fly Drosophila, the model plant Arabidopsis, the mouse, dog, chimpanzee, and, last but not least, humans. Researchers have also deduced the complete DNA sequences for a wide variety of human pathogens. These include the bacteria that cause cholera, tuberculosis, syphilis, gonorrhea, Lyme disease, and stomach ulcers, as well as hundreds of viruses—including smallpox virus and Epstein-Barr virus (which causes infectious mononucleosis). Examination of the genomes of these pathogens provides clues about what makes them virulent and will also point the way to new and more effective treatments.

Haemophilus influenzae (a bacterium that can cause ear infections and meningitis in children) was the first organism to have its complete genome sequence—all 1.8 million nucleotide pairs—determined by the shotgun sequencing method, the most common strategy used today. In the shotgun method, long sequences of DNA are broken apart randomly into many shorter fragments. Each fragment is then sequenced and a computer is used to order these pieces into a whole chromosome or genome, using sequence overlap to guide the assembly. The shotgun method is the technique of choice for sequencing small genomes. Although larger, more repetitive genome sequences are more challenging to assemble, the shotgun method—in combination with the analysis of large DNA fragments cloned in bacterial artificial chromosomes—has played a key role in their sequencing as well.

With new sequences appearing at a steadily accelerating pace in the scientific literature, comparison of the complete genome sequences of different organisms allows us to trace the evolutionary relationships among genes and organisms, and to discover genes and predict their functions. Assigning functions to genes often involves comparing their sequences with related sequences from model organisms that have been well characterized in the laboratory, such as the bacterium E. coli, the yeasts S. cerevisiae and S. pombe, the nematode worm C. elegans, and the fruit fly Drosophila.

Pharmaceutical Manufacturing and Administration

Pharmaceutical Solids. The term “drug” means, as stated in the Federal Food, Drug, and Cosmetic Act, a substance intended for use in the diagnosis, cure, mitigation, treatment, or prevention of disease in man or other animal. The term “biologic” means, as defined by the Food and Drug Administration, a subset of drugs that are distinguished by the biological manufacturing process. These definitions serve to differentiate a drug substance from a drug product. A drug product is the finished form, e.g., a parenteral drug product containing the drug substance. Efficacy studies are done to provide evidence of a drug's ability in diagnosis, cure, mitigation, treatment, or prevention of a disease. Diagnosis, cure, mitigation, treatment, or prevention does not mean 100% efficacy. Rather, diagnosis, cure, mitigation, treatment, or prevention means some level of efficacy, anywhere from 1% to 100%.

The discovery and development of new chemical entities (NCEs) into stable, bioavailable, marketable drug products is a long process. Due to the tremendous cost of developing a NCE, and industry's need to enhance productivity, it is desirable to create NCEs that have suitable physical-chemical properties, rather than compensate for deficiencies solely by the formulation process. Hence, property-based design can enhance the likelihood a NCE will have the desired physical-chemical properties that will facilitate its ability to be developed into a stable, bioavailable dosage form. Even so, well-designed preformulation studies are necessary to fully characterize molecules during the discovery and development process so that NCEs have the appropriate properties, and there is an understanding of the deficiencies that must be overcome by the formulation process.

Once a NCE is selected for development, choosing the molecular form that will be the active pharmaceutical ingredient (API) is the next milestone. Salt selection is the first API decision, in which absorption needs to be balanced with consistency of the API solid state. Excipients are the backbone of a formulation; they may be needed to stabilize the API.

A wide variety of different solid states are possible. Polymorphs exist when the drug substance crystallizes in different crystal packing arrangements all of which have the same elemental composition. Hydrates exist when the drug substance incorporates water in the crystal lattice. Desolvated solvates are produced when a solvate is desolvated and the crystal retains the structure of the solvate. Amorphous forms exist when a solid with no long range order and thus no crystallinity is produced.

Solutions, Emulsions, Suspensions, and Extracts. With regard to solutions, emulsions, suspensions, and extracts, the dosage forms are prepared by employing pharmaceutically and therapeutically acceptable vehicles. The active ingredient(s) may be dissolved in aqueous media, organic solvent or combination of the two, by suspending the drug (if it is insoluble) in an appropriate medium, or by incorporating the medicinal agent into one of the phases of an oil and water emulsion. These dosage forms can be formulated for different routes of administration: orally, introduction into body cavities, or external application.

A solution is a homogeneous mixture that is prepared by dissolving a solid, liquid, or gas in another liquid and represents a group of preparations in which the molecules of the solute or dissolved substance are dispersed among those of the solvent. An emulsion is a two-phase system prepared by combining two immiscible liquids, in which small globules of one liquid are dispersed uniformly throughout the other liquid. The word “suspension” is defined as a two-phase system consisting of an undissolved or immiscible material dispersed in a vehicle (solid, liquid, or gas). Extraction, as the term is used pharmaceutically, involves the separation of medicinally active portions of plant or animal tissues from the inactive or inert components by using selective solvents in standard extraction procedures.

Formulation may influence the bioavailability and pharmacokinetics of drugs in solution, including drug concentration, volume of liquid administered, pH, ionic strength, buffer capacity, surface tension, specific gravity, viscosity and excipients. Emulsions and suspensions are more complex systems. Consequently, the bioavailability and pharmacokinetics of these systems may be affected by additional formulation factors such as surfactants, type of viscosity agent, particle size and particle-size distribution, polymorphism and solubility of drug in the oil phase.

Parenteral Preparations. With respect to parenteral preparations, parenteral dosage forms differ from all other drug dosage forms because they are injected directly into body tissue through the primary protective system of the human body, the skin, and mucous membranes. They must be exceptionally pure and free from physical, chemical, and biological contaminants. These requirements place a heavy responsibility on the pharmaceutical industry to practice current good manufacturing practices (cGMPs) in the manufacture of parenteral dosage forms and upon pharmacists and other health care professionals to practice good aseptic practices (GAPs) in dispensing them for administration to patients.

Certain pharmaceutical agents, particularly peptides, proteins, and antibodies, can only be given parenterally because they are inactivated in the gastrointestinal tract when given by mouth. Parenterally administered drugs are relatively unstable and generally highly potent drugs that require strict control of their administration to the patient. Because of the advent of biotechnology, parenteral products have grown in number and usage around the world.

Formulation principles require that parenteral drugs be formulated as solutions, suspensions, emulsions, liposomes, microspheres, nanosystems, and powders to be reconstituted as solutions. Since most liquid injections are quite dilute, the component present in the highest proportion is the vehicle. The vehicle for most parenteral products is water. The United States Pharmacopeia (USP) requires Water for Injection (WFI). Water-miscible vehicles have been used. Non-aqueous vehicles are another alternative, the most important group being fixed oils. The USP permits substances to be added to a preparation to improve or safeguard its quality, for example, antimicrobial agents, buffers, antioxidants, tonicity agents, and cryoprotectants and lyoprotectants.

Drug pharmacokinetics, solubility, stability, and compatibility with additives dictate the choice of the final formulation of a parenteral drug. So do routes of administration. Injections may be administered by routes such as intravenous, subcutaneous, intradermal, intramuscular, intraarticular, and intrathecal. The type of dosage form (solution, suspension, etc.) will determine the particular route of administration that may be employed. Conversely, the desired route of administration will place requirements on the formulation.

In the preparation of a parenteral product, the general manufacturing process entails procurement, processing, packaging, and QA/QC. Procurement encompasses selecting and testing of the raw-material ingredients and containers. Processing includes cleaning the equipment, compounding the solution (or other dosage form), filtering the solution, sterilizing the containers, filling measured quantities of product into sterile containers, stoppering, freeze-drying, terminal sterilization, and sealing of the filled container. Packaging constitutes the labeling and cartoning of filled and sealed containers. The quality assurance and control unit is responsible for assuring and controlling the quality of the product through the process.

Ophthalmic Preparations. Ophthalmic preparations are specialized dosage forms designed to be instilled onto the external surface of the eye (topical), administered inside (intraocular) or adjacent (periocular) to the eye, or used in conjunction with an ophthalmic device. The preparations may have any of several purposes, therapeutic or prophylactic. Topical dosage forms have customarily been restricted to solutions, suspensions, and ointments, but have been expanded to include gels and inserts. The target is usually a tissue of the eye. Ophthalmic use imposes particle size, viscosity, and sterility specifications.

Medicated Topicals. The application of medicinal substances to the skin or various body orifices is a concept taking many forms. Medications are applied to the skin or inserted into body orifices (e.g., rectum, vagina, urethra) in liquid, semisolid, or solid form. Drugs are applied to the skin to elicit an effect on the skin surface, an effect within the stratum corneum, an effect requiring penetration into the epidermis and dermis, or a systemic effect.

Some topical dosage forms are ointments, transdermal drug delivery systems, suppositories, and others. Ointments are semisolid preparations intended for external application to the skin or mucous membranes. The USP recognizes four general classes of ointment bases: hydrocarbon bases, absorption bases, water-removable bases, and water-soluble bases. Transdermal drug delivery systems, like patches, increase skin residence times from hours to days to permit systemic uptake of the drug or drugs incorporated therein. Suppositories are solid dosage forms for insertion into the rectum, vagina, or urethra. Poultices, pastes, powders, dressings, creams, and plasters are sometimes intended for topical application.

Oral Solid Dosage Forms. Drug substances most frequently are administered orally by means of solid dosage forms such as tablets and capsules, although powders can also be administered as the simplest dosage form. Large-scale production methods used for the preparation of tablets and capsules usually require the presence of other materials in addition to the active ingredients. Additives also may be included in the formulations to facilitate handling, enhance the physical appearance, improve stability, and aid in the delivery of the drug to the bloodstream after administration.

Tablets may be defined as solid pharmaceutical dosage forms containing drug substances with or without suitable diluents and have been traditionally prepared by either compression, or molding methods. Recently, punching of laminated sheets, electronic deposition methods, and three-dimensional printing methods have been used to make tablets.

Compressed tablets are formed by compression and in their simplest form, contain no special coating. They are made from powdered, crystalline, or granular materials, alone or in combination with binders, disintegrants, controlled-release polymers, lubricants, diluents, and in many cases colorants. The vast majority of tablets commercialized today are compressed tablets, either in an uncoated or coated state.

In addition to the active or therapeutic ingredient, tablets contain a number of inert materials. The latter are known as additives or excipients. They may be classified according to the part they play in the finished tablet. The first group contains those that help to impart satisfactory processing and compression characteristics to the formulation. These include diluents (e.g., dicalcium phosphate, calcium sulfate, lactose, cellulose, kaolin, mannitol, sodium chloride, dry starch, and powdered sugar), binders (e.g., starch, gelatin, sugars, gums, cellulosics, and polyvinylpyrrolidone), glidants (e.g., colloidal silicon dioxide), and lubricants (e.g., talc, magnesium stearate, calcium stearate, stearic acid, glyceryl behanate, hydrogenated vegetable oils, and polyethylene glycol). The second group of added substances helps to give additional desirable physical characteristics to the finished tablet. Included in this group are disintegrants (e.g., starches, clays, celluloses, algins, gums, and cross-linked polymers), surfactants, colors, and, in the case of chewable tablets, flavors, and sweetening agents, and in the case of controlled-release tablets, polymers or hydrophobic materials, such as waxes or other solubility-retarding materials. In some cases, antioxidants or other materials can be added to improve stability and shelf-life.

Capsules are solid dosage forms in which the drug substance is enclosed in either a hard or soft, soluble shell of a suitable form of gelatin. Compared with tablets, powders for filling into hard gelatin capsules require a minimum of formulation efforts. The powders usually contain diluents such as lactose, mannitol, calcium carbonate, or magnesium carbonate. Lubricants such as the stearates also are used frequently. The gelatin for soft shell capsules is plasticized typically by the addition of glycerin, sorbitol, or a similar polyol.

Controlled Drug Delivery. Controlled drug delivery can be defined as delivery of the drug at a predetermined rate and/or to a location according to the needs of the body and disease states for a definite time period. In rate-controlled release systems, the mechanism by which the release rate is controlled is diffusion, dissolution, osmosis, mechanically driven pump, swelling, erosion, and stimulation. In targeted delivery systems, targeting is achieved by colloidal drug carriers, ligand-mediated targeting, resealed erythrocytes, bioadhesives, and prodrugs. Device implantation, encapsulated cells, and reservoir microchips are new delivery systems being developed.

Currently, most modified-release delivery systems fall into the following three categories: Delayed-release, extended-release, and site-specific and receptor targeting. Delayed-release systems are either those that use repetitive, intermittent dosing of a drug from one or more immediate-release units incorporated into a single dosage form, or an enteric delayed release system. Extended-release systems include any dosage form that maintains therapeutic blood or tissue levels of the drug for a prolonged period. Site-specific and receptor targeting refers to targeting a drug directly to a certain biological location, a site in the former case, a receptor in the latter case. Recently, a novel modification of drug delivery systems has emerged from the pharmaceutical industry, in which a fast-dissolve drug delivery system consists of a solid dosage form that dissolves or disintegrates in the oral cavity without the need of water or chewing.

Aerosols. Inhalation therapy has been used for many years, and there has been a resurgence of interest in delivery of drugs by this route of administration. The number of new drug entities delivered by the inhalation route has increased over the past 5 to 10 years. This type of therapy also has been applied to delivery of drugs through the nasal mucosa, as well as through the oral cavity for buccal absorption. Originally, this type of therapy was used primarily to administer drugs directly to the respiratory system (treatment of asthma); inhalation therapy is now being used for drugs to be delivered to the bloodstream and finally to the desired site of action. Drugs administered via the respiratory system (inhalation therapy) can be delivered either orally or nasally. Further, these products can be developed as a nebulizer/atomizer, dry powder inhaler, nasal inhaler, or metered dose aerosol inhaler.

Biotechnology Drugs. Recombinant human proteins have been made possible through the biotechnology techniques that allow the production of normally minute amounts of proteins, particularly by use of the DNA that encodes the protein. In antisense RNA and DNA, a short oligonucleotide (10-20 base pairs) complementary to a specific mRNA binds to its target mRNA, which inhibits protein translation by interfering with ribosomal function; additionally, the resulting DNA-RNA duplex recruits the activity of RNase H, a ubiquitous enzyme that degrades the RNA itself. With ribozymes, RNAs possess an enzymatic RNA-degrading activity and are directed toward a specific RNA by the sequence similarity used by antisense molecules. Aptamers are RNA molecules specifically selected by virtue of their three dimensional nature for high affinity to certain molecular targets. Small interfering RNAs are small RNA molecules that interfere with expression of genes by a mechanism where a type III RNase enzyme (called “Dicer”) is activated to cleave long RNA molecules into 21-28 base pair fragments which then hybridize to other copies of long RNA molecules to catalyze their degradation. In gene therapy, a gene is introduced into the body to help fight a disease. In general, the DNA encoding this gene is encoded on a plasmid molecule or is part of a viral vector that can infect cells with the appropriate desirable gene without causing viral disease. Delivery methods for these gene sources usually either exploit the DNA delivery tactic of the virus itself or employ cationic liposomal complexes with the DNA to mask the plasmid's negative charge.

New Drug Process. New drug development can proceed along varied pathways for different compounds, but a development paradigm has been articulated that has long served well as a general model. In outline form, the paradigm portrays new drug discovery and development as proceeding in a sequence of (possibly overlapping) phases. Discovery programs result in the synthesis of compounds that are tested in preclinical tests called assays and animal models. Clinical (human) testing typically proceeds through three successive phases. In phase I, a small number of usually healthy volunteers are tested to establish safe dosages and to gather information on the absorption, distribution, metabolic effects, excretion, and toxicity of the compound. To conduct clinical testing in the United States, a manufacturer must first file an investigational new drug application (IND) with the Food and Drug Administration (FDA). Phase II trials are conducted with subjects who have the targeted disease or condition and are designed to obtain evidence on safety and preliminary data on efficacy. The number of subjects tested in this phase is larger than in phase I and may number in the hundreds. The final pre-approval clinical testing phase, phase III, typically consists of a number of large-scale (often multi-center) trials that are designed to firmly establish efficacy and to uncover side-effects that occur infrequently. The number of subjects in phase III trials for a compound can total in the thousands. Once drug developers believe that they have enough evidence of safety and efficacy, they will compile the results of their testing in an application to regulatory authorities for marketing approval. In the United States, manufacturers submit a new drug application (NDA) or a biological license application (BLA) to the FDA for review and approval.

Dosages. The dose of a drug required to produce a specified effect in 50% of the population is the median effective dose, abbreviated ED50. In preclinical studies of drugs, the median lethal dose, as determined in experimental animals, is abbreviated as the LD50. The ratio of the LD50 to the ED50 is an indication of the therapeutic index, which is a statement of how selective the drug is in producing the desired versus its adverse effects. Drugs that exhibit high therapeutic indices are preferred.

SAA Increases Cell Proliferation in In Vitro Suppression Assays.

In SOME embodiments, SAA induces regulatory T cells (Treg) suppression of the proliferation of effector T cells (Teff) in standard suppression assays. In one example of a suppression assay, Tregs are cultured with Teffs, plate-bound anti-CD3, in the presence of irradiated CD3-depleted peripheral blood mononuclear cells (PBMC). The addition of SAA, but not a control, to suppression assays stimulates cell proliferation. Dose-response experiments over a range of concentrations of SAA show that the effect increases and reaches a plateau. The effect of SAA on cell proliferation is irreversible after 48-hour exposure of cells to the SAA. In addition, SAA does not induce or suppress cell proliferation in stimulation assays, in which only one type of T cell (Treg or Teff) is cultured with platebound anti-CD3 and irradiated APC.

In some embodiments, CD4+ T cells are purified by negative selection from buffy coats. The CD4+ T cell fraction is then incubated with anti-CD25 microbeads to isolate CD4+CD25+ cells. The flow-through fraction after magnetic purification contains CD4+CD25− Teff. Other embodiments focus on the use of flow cytometry to sort regulatory T cells based on a combination of markers, such as CD4+, CD25+, and CD127Io/−, on the reasoning that CD127 is inversely correlated with Foxp3 expression in humans. Purity of sorted cells is confirmed by Foxp3 staining.

In some embodiments, standard 3H-thymidine-based suppression assays are performed to analyze Treg function. For example, autologous Treg and Teff are cultured together with allogeneic irradiated CD3-depleted peripheral blood mononuclear cells (antigen presenting cells or APC). Anti-CD3 antibodies are pre-coated on well plates before suppression assays. Cells are pulsed with 3H-thymidine and harvested. 3H-thymidine incorporation is determined using a liquid scintillation counter. Parallel suppression assays with all autologous cell types (Treg, Teff, and APC) are also performed, in which Treg show similar suppressive potential compared to assays with allogeneic APC. Stimulation assays are set up similarly with allogeneic irradiated APC and only one type of T cell (either Treg or Teff).

In some embodiments, for suppression assays without APC (below), anti-CD3 antibodies are plate-bound in well plates. Soluble anti-CD28 is added, followed by Teff and Treg. 3H-thymidine is added to each well, and the plate is harvested, as described for standard suppression assays. In some embodiments, carboxyfluorescein diacetatesuccinimidyl ester (CFSE) dilution assay is used for measuring T cell proliferation in suppression assays (below). Briefly, Treg are labeled with CFSE. Assays with labeled cells are performed as described for 3H-thymidine-based suppression assays. Cells are harvested and dye dilution is analyzed by flow cytometry. Stimulation assays are set up similarly with allogeneic irradiated APC and only one type of CFSE-labeled T cells (either Treg or Teff). For APC fixation (below), these cells are first re-suspended in paraformaldehyde. Fixed cells are pelleted by centrifugation, washed and then cultured.

SAA Selectively Induces Proliferation of CD4+CD25+ Treg.

In some embodiments, carboxyfluorescein diacetate succinimidyl ester (CFSE) assays reveal that in suppression assays with SAA, the Treg, but not Teff population is dividing. SAA-exposed Treg continue to suppress the proliferation of Teff, as indicated by reduced CFSE dilution in Teff in suppression assays with SAA compared to stimulation assays with SAA and Teff alone. This indicates Treg gain proliferative potential without loss of suppressive capacity in the presence of SAA.

SAA Induces Treg Proliferation In Vivo.

In some embodiments, SAA induces Treg proliferation in vivo. For example, SAA is administered by intraperitoneal injection, and a significant increase in Treg abundance in the peritoneal cavity of SAA-injected subjects is observed compared to those injected with a control. Furthermore, a significantly higher percentage of peritoneal Treg from the subject express the nuclear antigen Ki-67, indicating that they are undergoing cell division. In contrast, SAA injection does not enhance Teff proliferation in vivo.

Monocytes are Necessary for the Induction of Treg Proliferation Mediated by SAA.

In some embodiments, suppression assays are performed with anti-CD3 and anti-CD28 antibodies as T cell stimuli in the presence or absence of antigen presenting cells (APC). In the absence of APC, SAA fails to induce Treg proliferation, indicating that cells in the APC mixture mediate the mitogenic effect of SAA on Treg. In some embodiments, monocytes or B cells, the two more abundant professional APC subsets in peripheral blood, are depleted. Depleting monocytes leads to a significant decrease in cell proliferation in suppression assays with SAA. In contrast, depletion of B cells fails to abrogate the increased proliferation in suppression assays with SAA. In some embodiments, purified monocytes or B cells are added to the culture with SAA. Adding purified monocytes with SAA is sufficient to induce Treg cell proliferation at similar levels to those observed using unfractionated APC. In contrast, adding purified B cells with SAA is not able to enhance Treg cell proliferation. In some embodiments, monocytic cells are depleted in vivo with clodronate liposomes. Intraperitoneal injection of clodronate liposomes leads to systemic depletion of monocytic cells. In the absence of monocytic cells, there are significant decreases in Treg abundance and expression of Ki-67 in the peritoneal cavities of SAA-treated animals. This indicates monocytes are indispensable for the mitogenic effects of SAA on Treg.

SAA Stimulates IL-1 and IL-6 Production by Monocytes.

In some embodiments, fixation of the APC block their ability to support Treg proliferation in the presence of SAA in suppression assays, indicating soluble factors derived from cells in the APC mixture are involved in the reversal of Treg anergy. Levels of two proinflammatory cytokines, IL-1 and IL-6, are significantly elevated in suppression assays with SAA compared to assays with a control after 24 hours in culture. SAA treated APC or monocytes, in particular, produces significantly higher levels of IL-1 and IL-6, compared to cells that are exposed to a control. Monocytes are found to express significantly higher levels of SAA receptors than B cells and produce significantly higher levels of IL-1 and IL-6 upon stimulation with SAA. In some embodiments, to track possible differentiation of CD14+ monocytes into myeloid dendritic cells (DC), CD14+ monocytes are isolated from APC, labeled with CFSE and added back to the APC mixture. In the harvested population of APC, two subsets of DC (Lineage− DR+cells) are found: one that does not stain for CFSE (blood DC from initial APC) and another that is positive for CFSE (monocyte-derived DC). This indicates that SAA induces monocyte differentiation into DC. Significant increases of DR and CD40 on DC are found in assays with SAA compared to a control. The expression of other markers is not significantly different on DC in suppression assays with SAA compared to a control. This surface phenotype of SAA induced DC most resembles that of immature DC.

IL-1 and IL-6 are Necessary for the Reversal of Treg Anergy.

In some embodiments, suppression assays are performed with SAA in the presence of IL-1 receptor antagonist or blocking antibody against IL-6 at various concentrations. Each of these reagents mediates significant inhibition of SAA-induced Treg proliferation. Blocking other inflammatory cytokines, such as TNF-α, does not significantly alter SAA effects on cell proliferation. Without ruling out that other cytokines, growth factors, or cell-cell contact also contribute, this indicates indispensable roles for IL-1 and IL-6 in the in vitro reversal of Treg anergy by SAA.

IL-1 and IL-6 have Distinct Effects on Mitogenic Pathways in Treg.

In some embodiments, Treg are found to selectively exhibit increased activation of AKT and ERK1/2, signaling molecules that have been implicated in mitogenic processes, in assays with SAA compared to assays with a control. In addition, phosphorylated STAT3, a signaling molecule downstream of IL-6, is also found to be expressed at higher levels in Treg, but not in Teff, in suppression cultures with SAA compared to a control. Treg are also found to express significantly higher levels of phosphorylated AKT, ERK1/2, and STAT3 than Teff in the same suppression cultures with SAA. In some embodiments, the activation of mitogenic pathways in the presence of IL-1 receptor antagonist and neutralizing antibody against IL-6 is analyzed in assays with SAA in an attempt to link IL-1 and IL-6 to the activated mitogenic pathways in proliferating Treg. Blocking IL-1 or IL-6 is found to abrogate the increased expression of phosphorylated ERK1/2 in Treg. Blocking IL-1, but not IL-6, is found to inhibit phosphorylation of AKT in Treg. Blocking IL-6 is also found to abrogate STAT3 activation in Treg. This indicates that IL-1 and IL-6 are necessary for the activation of mitogenic pathways in Treg and Teff and also reveals differential activation of these pathways by these cytokines.

Treg Exhibit Lower Expression of SOCS3 Compared to Teff.

In some embodiments, the expression of surface receptors for IL-1 and IL-6 on Treg and Teff is measured to investigate the mechanism that is responsible for the selective activation of mitogenic signaling pathways in response to IL-1 and IL-6 by Treg. IL-1R1 (the high affinity IL-1 receptor) and the signaling component of IL-6 receptor complex (gp130) are found to be expressed at low levels in Treg and Teff in suppression assays. However, expression of these molecules on Treg and Teff is up-regulated during the course of the assay. Furthermore, higher expression of IL-1 and IL-6 receptors on both Treg and Teff is induced in the presence of SAA compared to a control, despite differential responsiveness of these cell types to these cytokines. In some embodiments, SOCS3, a suppressor of cytokine signaling proteins and a chief regulator of both IL-1 and IL-6 signaling, is found to be expressed at significantly lower levels in Treg compared to Teff, providing a possible explanation for these seemingly paradoxical results that Treg and Teff differ in regulatory mechanisms that control IL-1 and IL-6 intracellular signaling.

Induction of SOCS3 in Treg Inhibits their Selective Proliferation Driven by SAA.

In some embodiments, SOCS3 expression is modulated in Treg using forskolin, a reagent known to induce SOCS3 expression in various cell types, to test the hypothesis that the difference in SOC3 expression in Treg and Teff regulates their selective response to IL-1 and IL-6. Freshly isolated Treg are cultured with different concentrations of forskolin and their expression of SOCS3 is examined at different time points. Intracellular staining shows an increase in SOCS3 expression by forskolin-treated Treg. SOCS3 expression by Treg, initially induced by forskolin at conditions determined to be optimal for dosing, remains at a level comparable to that in Teff during the suppression assays. Forskolin-treated Treg still suppress Teff proliferation, but they no longer proliferate in response to SAA. Similar results are obtained with SOCS3 transfection in Treg. This indicates that the level of expression of SOCS3 by Treg regulates the dynamic range of their proliferative response to SAA in suppression assays.

Induction of SOCS3 in Treg Abrogates their Selective Activation of Mitogenic Signaling Driven by SAA.

In assays with forskolin-treated Treg, levels of pERK1/2 and pSTAT3 in Treg are significantly reduced, and activation of AKT is abrogated. Increased activation of mitogenic signaling in Treg compared to Teff cocultured in the same assays with SAA is either abrogated (for ERK1/2 and AKT) or reversed (Teff become more activated with respect to STAT3) when forskolin-treated Treg are used. Forskolin-treated Treg also show a significant decrease in activation of ERK1/2, AKT, and STAT3 compared to untreated Treg that are exposed to the same SAA in suppression assays. Conversely, in the presence of forskolin-treated Treg, activation of ERK1/2 and STAT3 is modestly enhanced in Teff in assays with SAA compared to those with a control. Teff co-cultured with forskolin-treated Treg also show a modest increase in ERK1/2 and STAT3 activation compared to those cocultured with untreated Treg. This indicates Treg-specific modulation of SOCS3 expression tunes the competitive fitness between Treg and Teff in response to SAA induced activation of mitogenic pathways.

### Reagents, Devices and Kits

Also provided are reagents, devices and kits thereof for practicing one or more of the above-described methods. The subject reagents, devices and kits thereof may vary greatly.

Reagents, devices and kits of interest include those mentioned above with respect to the methods of inducing regulatory T cell proliferation and methods of treating autoimmune or rheumatoid diseases with regulatory T cells. For example, kits of interest include those mentioned above for stimulating regulatory T cell proliferation in vivo and methods of treating autoimmune or rheumatoid diseases by stimulating regulatory T cell proliferation in vivo. Such kits may include SAA compositions, buffers for their delivery, other known anti-autoimmune therapies, etc. As another example, kits of interest include those for preparing regulatory T cells ex vivo and methods of treating autoimmune or rheumatoid diseases with regulatory T cells prepared ex vivo. Such kits may include one or more of the following: media suitable for culturing leukocytes, SAA polypeptides, antigen presenting cells, reagents for selecting regulatory T cells, buffers for delivering regulatory T cells to individuals, etc.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

To dissect effects of the inflammatory milieu on regulatory T cells (Treg), we sought to simulate this environment in vitro using plasma from children with systemic juvenile idiopathic arthritis (SJIA). SJIA is an often chronic, auto-inflammatory disease, characterized by arthritis and systemic inflammation. During active disease, elevated levels of pro-inflammatory cytokines and acute phase reactants are present locally in synovial fluid and systemically in plasma (Kutukculer, N., Caglayan, S. & Aydogdu, F., Clin. Rheumatol. 1988, 17, 288-292; van den Ham, H. J., de Jager, W, Bijlsma, J. W., Prakken, B. J. & de Boer, R. J., Rheumatology (Oxford) 2009, 48, 899-905). We hypothesized that circulating mediators in this disease might modulate Treg activity. We utilized a standard suppression assay to evaluate the influence of SJIA plasma on Treg function and to elucidate the interplay between Treg, effector T cells (Teff), antigen presenting cells (APC), and the soluble factors elaborated by these cells after exposure to SJIA plasma. We identified serum amyloid A (SAA), an acute phase reactant previously known to rise to strikingly high levels in active SJIA (Miyamae, T. et al., Arthritis Res. Ther. 2005, 7, R746-55), as an endogenous factor that induces cellular and cytokine conditions supporting the expansion of Treg while maintaining their suppressive capacity.

### Methods

Human Subjects.

The study was approved by the Institutional Review Board at Stanford University. Subjects included children with SJIA (n=30) followed at Stanford Pediatric Rheumatology Clinic and age-and-ethnicity-matched immunologically healthy children (n=30) from the Stanford Pediatric Endocrinology Clinic. None of healthy control (HC) subjects had co-existing conditions such as diabetes, obesity, autoimmune disease, or inflammatory disorders. All subjects provided informed consent before participating in the study. Based on clinical data, we classified samples into two categories: active disease (flare) of either systemic/arthritis or both, or inactive disease. For inactive disease, subjects relying on medication to control disease activity were considered quiescent, while subjects off all drugs were considered in remission. In addition to blood samples from SJIA patients, buffy coats, derived from up to 450 ml whole blood from healthy adults, were obtained from the Stanford Blood Bank.

Plasma Preparation

Plasma was prepared from whole, anti-coagulated blood within 2 hours after blood draw. Whole blood samples were centrifuged at 25° C. at 514 g for 5 minutes to remove cells, and then underwent two additional rounds of centrifugation at 4° C. at 1730 g for 5 and 15 minutes respectively to remove platelets. Final plasma samples were stored at −80° C. until analysis.

Cell Isolation

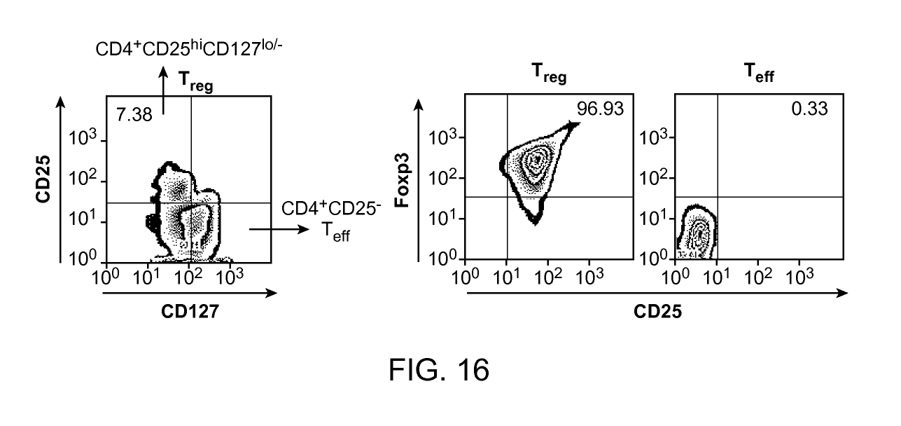

CD4+ T cells were purified with CD4+ Rosette Kit (Stemcell Technologies) from buffy coats. The CD4+ T cell fraction was then incubated with anti-CD25 microbeads (Miltenyi Biotech) to isolate CD4+CD25+ cells. The flow-through fraction after magnetic purification contained CD4+CD25− Teff. All procedures were performed according to manufacturers' standard protocols. CD4+CD25+ T cells were incubated with anti-CD127-APC, anti-CD25-PE, and anti-CD4-FITC antibodies (BD Biosciences) before undergoing flow cytometric sorting for CD4+CD25+CD127Io/− Treg and CD4+CD25+CD127+ activated Teff. Purity of sorted cells was confirmed to be higher than 95% by Foxp3 staining (eBioscience) (FIG. 16). Cells were rested for 2 hours in 37° C. incubator before being used in suppression assays.

T-Cell Stimulation and Treg Suppression Assays Using 3H-Thymidine Incorporation to Detect Cell Proliferation

Standard 3H-thymidine-based suppression assays were performed to analyze Treg function. Autologous Treg and Teff were cultured at 3,750 cells per 50 μl per well in complete media (RPMI+10% FBS+1% L-glutamine) with allogeneic irradiated CD3-depleted peripheral blood mononuclear cells (antigen presenting cells or APC), at 37,500 cells per 50 μl per well. Anti-CD3 antibodies (clone UCHT1, BD Biosciences) were pre-coated on U-bottom 96 well plates at 5 μg/ml for 4 hours at 37° C. before suppression assays. Additional media was added so the final volume in each well was 200 μl. On day 6, cells were pulsed with 1 μCi 3H-thymidine (25 μl) per well and harvested on day 7 with a Tomtec cell harvester. 3H-thymidine incorporation was determined using a 1450 microbeta Wallac Trilux liquid scintillation counter. Parallel suppression assays with all autologous cell types (Treg, Teff, and APC) were also performed, in which Treg showed similar suppressive potential compared to assays with allogeneic APC. Stimulation assays were set up similarly with allogeneic irradiated APC and only one type of T cell (either Treg or Teff). All assays were performed in triplicates. For suppression assays without APC, anti-CD3 antibodies at 5 μg/ml final concentration (clone UCHT1, BD Biosciences) were plate-bound in U-bottom 96-well plates for 4 hours at 37° C. Soluble anti-CD28 (5 μg/ml, BD Biosciences) was added, followed by Teff (40,000 cells per 50 μl per well) and Treg (40,000 cells per 50 μl per well). After 6 days, 1 μCi of 3H-thymidine was added to each well, and the plate was harvested, as described for standard suppression assays.

T-Cell Stimulation and Treg Suppression Assays Using Dye Dilution to Detect Cell Proliferation

Carboxyfluorescein diacetate succinimidyl ester (CFSE) dilution assay was used for measuring T cell proliferation in suppression assays. Briefly, Treg were labeled with CFSE using Cell Tracer CFSE Cell Proliferation kit (Molecular Probes) at a final concentration of 10 μM, according to manufacturer's instructions. Assays with labeled cells were performed as described for 3H-thymidine-based suppression assays. After 7 days of culture, cells were harvested and dye dilution was analyzed by flow cytometry. Stimulation assays were set up similarly with allogeneic irradiated APC and only one type of CFSE-labeled T cells (either Treg or Teff). For APC fixation, these cells were first re-suspended in PBS+10% FBS+1% paraformaldehyde for minutes at 37° C. Fixed cells were pelleted by centrifugation, washed and then cultured in complete media.

Suppression Assays with Addition of SJIA, HC Plasma

To evaluate effects of plasma on suppression assays and immune cell cultures, frozen plasma samples were thawed at 25° C. and debris were removed with sterile 40 μm filters (BD Biosciences). All plasma samples were tested in duplicates or triplicates. To control for variations in suppressive and proliferative potentials of Treg and Teff, respectively, both HC and SJIA plasma samples were used in parallel suppression assays with the same set of purified cells for each round of experiments. In addition, fold change in 3H-thymidine cpm in assays with plasma compared to those in complete media alone, was computed to analyze the effects of plasma in suppression assays or stimulation assays. In CFSE assays, percentage of proliferating cells (shown by dye dilution) was used to analyze the effects of plasma on cell proliferation. IL-1Ra, neutralizing antibody against IL-6 and TNF-α (R&D System), and recombinant IL-1, IL-6 (Peprotech) were used to evaluate the roles of these cytokines in suppression assays. Recombinant SAA (Peprotech) was used at different concentrations in suppression assays in the presence of polymixin (5 μg/ml). For plasma wash-out experiments, suppression assays were performed in the presence of SJIA plasma in V-bottom 96-well plates. At indicated days, cells were spun down and supernatant was removed and replaced with fresh complete media. To pharmacologically modulate SOCS3, isolated Treg were cultured with forskolin (Calbiochem) at different concentrations and analyzed for SOCS3 expression at different time points.

Detection of Secreted Cytokines in Suppression Assays and Plasma Samples.

Both suppression assays and APC cultures were set up for supernatant collection. For APC cultures, 50 μl of APC stock solution (750,000 cells per ml) were incubated in a final volume of 200 μl per well in complete media. Plasma samples were used at 35 μl per well. At different time points, supernatants were collected after centrifugation to pellet cells; supernatants representing samples from the same experimental conditions were pooled from 6 wells of 96-well plates and stored at −80° C. until analysis. To detect cytokines in the supernatants, cytometric bead arrays (BD Biosciences) and ELISA (R&D System) were used according to manufacturers' protocols. Similar assays were performed for plasma samples from HC and SJIA subjects.

Characterization of Plasma Proteins.

Plasma samples were heat inactivated at 100° C. forminutes. Dialysis was performed with 5 kD dialysis membranes (GE Healthcare). Albumin depletion was performed with HiTrap Blue HP columns (GE Healthcare). Plasma samples were diluted 1:4 with binding buffer (20 mM sodium phosphate buffer, pH 7.0) before being passed through the columns. Flow-through fraction was size-fractionated by Superdex 200 gel filtration column (GE Healthcare) using 20 mM sodium phosphate buffer, pH 7.0, 150 mM NaCl. All chromatographic assays were performed according to the manufacturers' instructions. Depletion of SAA from plasma samples was performed with anti-SAA antibodies (Santa Cruz Biotechnology) via immunoprecipitation for 4 consecutive rounds. Negative control for depletion experiments was performed with L243 (anti-HLA-DR) antibodies.

In Vivo Effects of SAA on Treg Proliferation

C57B6 mice (male, 8 to 10 weeks old) were purchased from Jackson Laboratory. Mice were injected intraperitoneally with recombinant human SAA (Peprotech, 30 μg in 100 μl PBS), purified human serum albumin (Sigma Aldrich, 30 μg in 100 μl PBS), or endotoxin (Sigma Aldrich, 0.25 ng in 100 μl PBS). Animals were sacrificed 16 hours later; peritoneal cells were harvested and stained for surface and intracellular markers to detect Treg frequency and proliferation. In vivo depletion of monocytes was performed with clodronate liposomes (Encapsula). In these experiments, 400 μl of clodronate or empty liposomes were injected intraperitoneally 24 hours before SAA injection.

Flow Cytometry

Detection of surface markers and intracellular molecules was performed. Antibodies used in these experiments included anti-human RAGE, SOCS3 (Abcam), CD14-FITC, DR-FITC, Foxp3-FITC, Lin (mixture of CD3, CD14, CD16, CD19, CD56)-FITC, CD14-PE, CD25-PE, CD40-PE, CD83-PE, CD127-PE, HVEM-PE, PDL2-PE, IL-1-PE, IL-6-PE, CD3-PerCP, CD4-PerCP, CD19-PerCP, CD25-PerCP, DR-PerCP, Lin-PerCP, CD3-APC, CD4-APC, CD14-APC, CD36-APC, CD86-APC, TLR2-APC, TLR4-APC, PDL1-APC, CCR7-APC, IL-6-APC, Lin-APC (Biolegend), Ki-67 FITC, pSTAT3 Alexa-647, pAKT Alexa-647, pERK1/2 Alexa-647 (BD Biosciences), and FPRL-1-APC(R&D System). Antibodies to mouse proteins used in these experiments included anti-CD25-PE, CD4-PerCP, CD11b-PerCP, CD115-APC, and Foxp3-APC (Biolegend). For in vitro experiments, to assay for expression of different surface and intracellular molecules in a specific cell subset (Treg, Teff or APC) in mixed cell cultures, the relevant subset was labeled with CFSE prior to suppression assays. Cells were pelleted at various time points and underwent standard staining protocols of the manufacturers. For in vivo experiments, cells were harvested from the peritoneal cavity and underwent flow cytometric analysis.

Statistical Analysis

All statistical procedures were performed with Prism software (Graph Pad). Data were tested for normality (Koromonov-Smirnov's test) and variance equality (Bartlett's test) before being subjected to appropriate statistical tests. Differences with p<0.05 were considered statistically significant. Correction for multiple comparisons was performed via Bonferroni method.

### Results

SJIA Plasma Increases Cell Proliferation in In Vitro Suppression Assays.

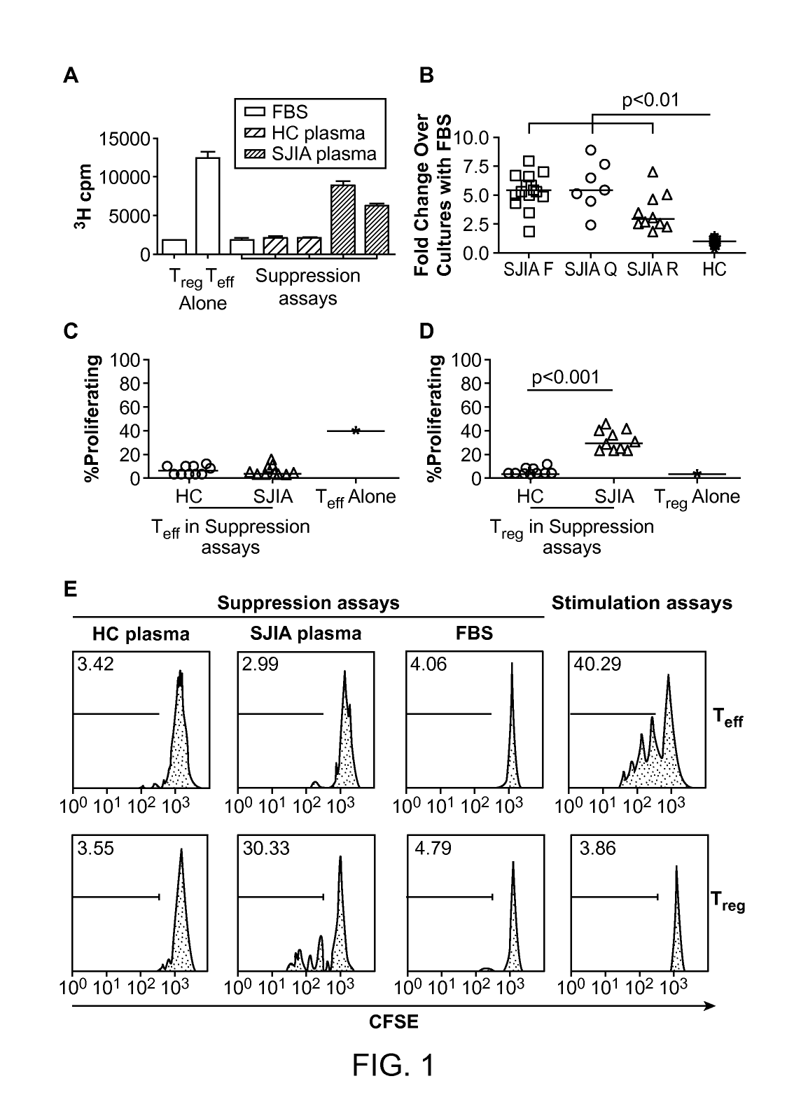

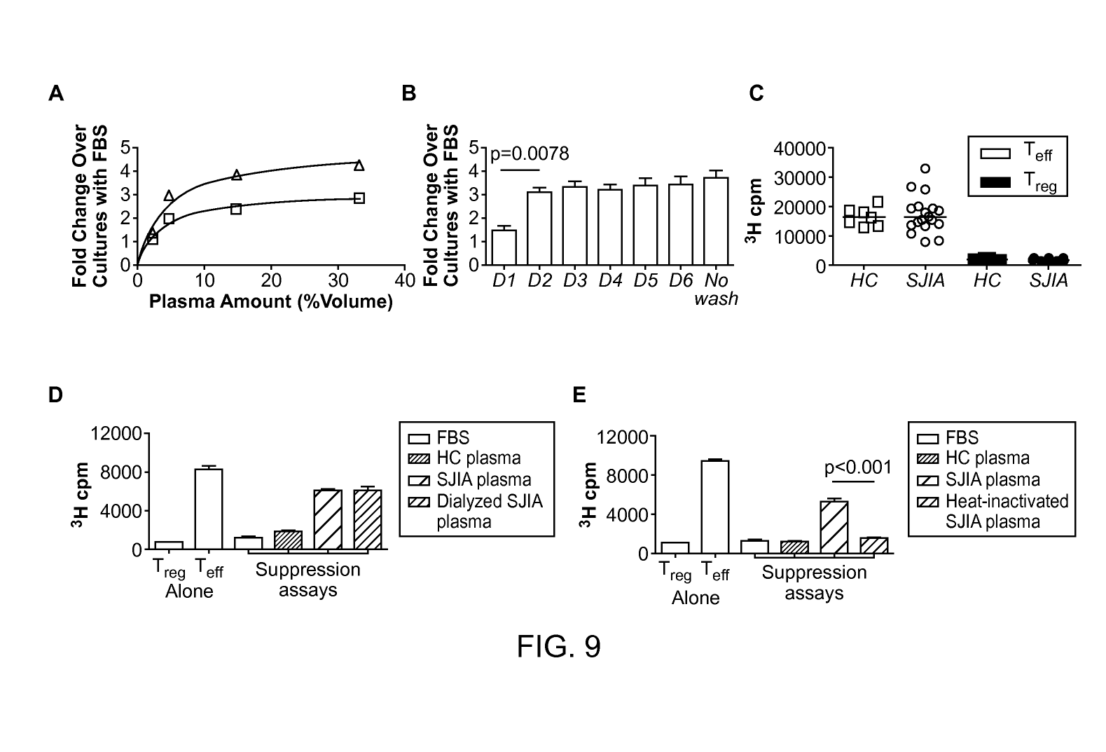

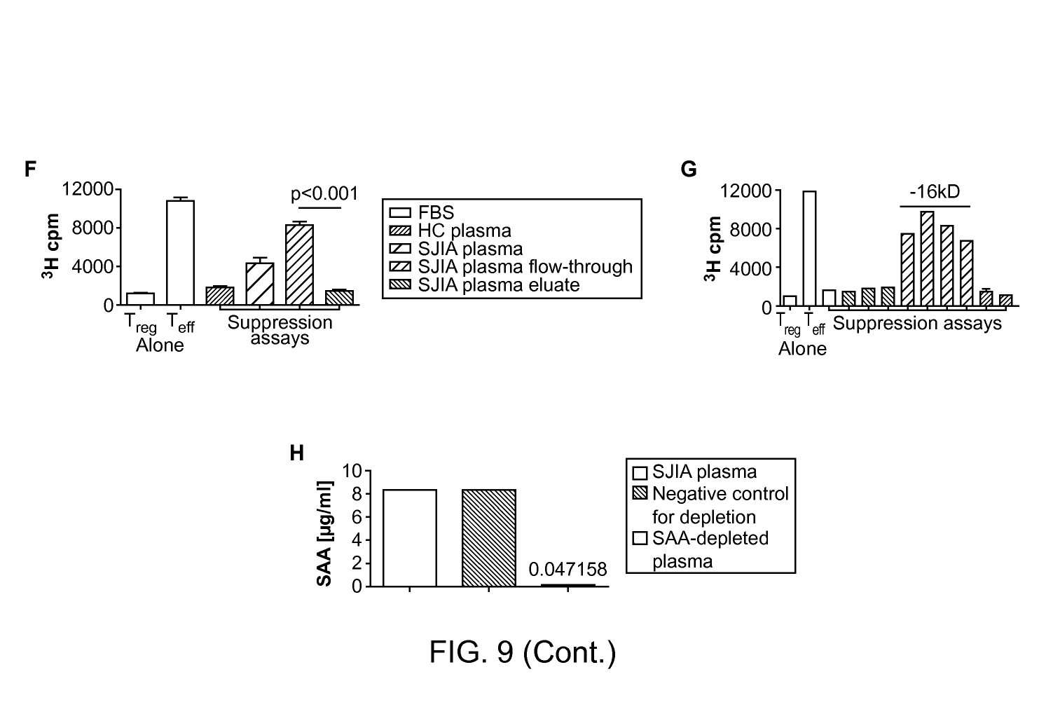

Regulatory T cells (Treg) suppressed the proliferation of effector T cells (Teff) in standard suppression assays, in which Treg were cultured with Teff, plate-bound anti-CD3, and irradiated CD3-depleted peripheral blood mononuclear cells (PBMC), used as antigen presenting cells (APC) (FIG. 1A). Initial studies indicated that addition of SJIA plasma (15% by volume), but not healthy control (HC) plasma, to suppression assays, stimulated cell proliferation (FIG. 1A). Dose-response experiments over a range of 5 μl (2.4% by volume) to 100 μl (33.3%) plasma showed that the effect increased and reached a plateau at 35 μl, the dose we used for further studies (FIG. 9A). The effect of SJIA plasma on cell proliferation was irreversible after 48-hour exposure of cells to the plasma (FIG. 9B). Strikingly, this effect could be mediated by plasma samples from SJIA subjects with various degrees of disease activity, i.e., flare, quiescence (inactive disease on medication), or remission (inactive disease off medication) (FIG. 1B). In addition, SJIA plasma did not induce or suppress cell proliferation in stimulation assays, in which only one type of T cell (Treg or Teff) was cultured with plate-bound anti-CD3 and irradiated APC (FIG. 9C).

SJIA Plasma Selectively Induces Proliferation of CD4+CD25+ Treg.

Because Treg are known to be anergic, it initially seemed likely that the proliferating population in suppression assays with SJIA plasma was Teff. To determine which cell population was proliferating, we used flow cytometry to track carboxyfluorescein diacetate succinimidyl ester (CFSE)-labeled, dividing cells. Surprisingly, CFSE assays evealed that the Treg, but not Teff, population in suppression assays with SJIA plasma was dividing (FIG. 1C-E). SJIA plasma-exposed Treg continued to suppress the proliferation of Teff, as indicated by reduced CFSE dilution in Teff in suppression assays with SJIA plasma compared to stimulation assays with SJIA plasma and Teff alone (FIGS. 1C, E). Altogether, these results indicated that Treg gained proliferative potential without loss of suppressive capacity in the presence of SJIA plasma.

Serum Amyloid a is Elevated in SJIA Plasma and Necessary for its Mitogenic Effects on Treg.

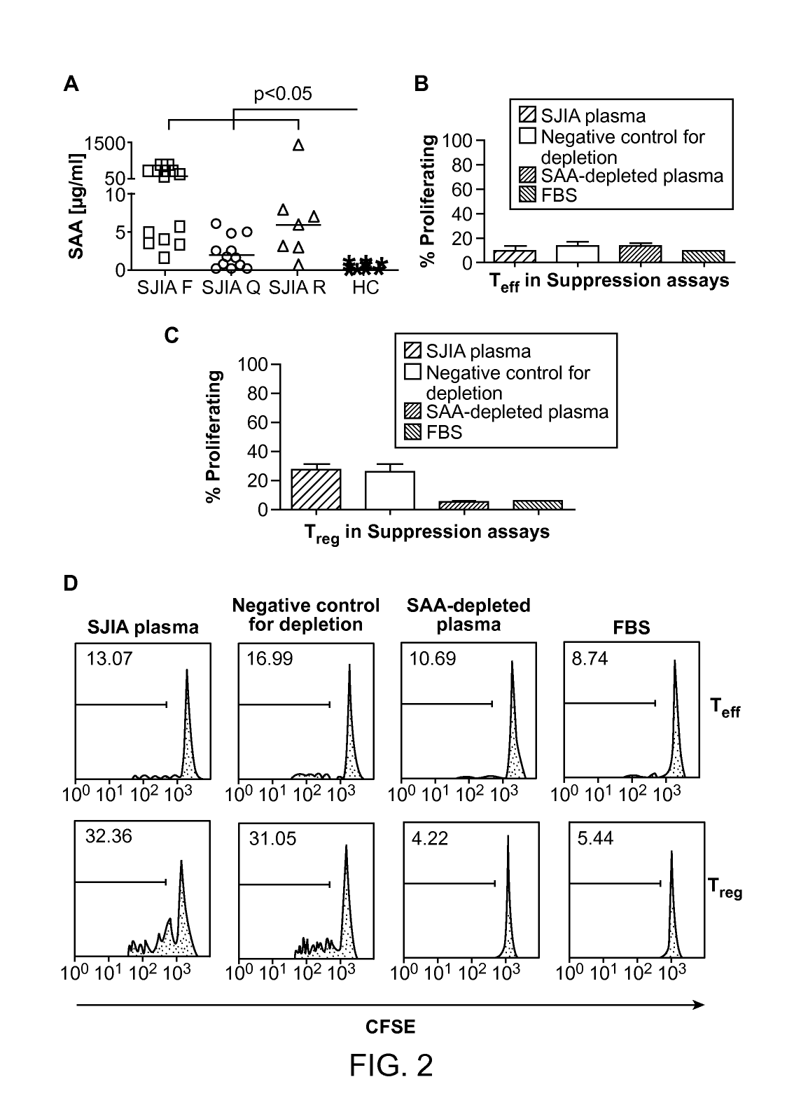

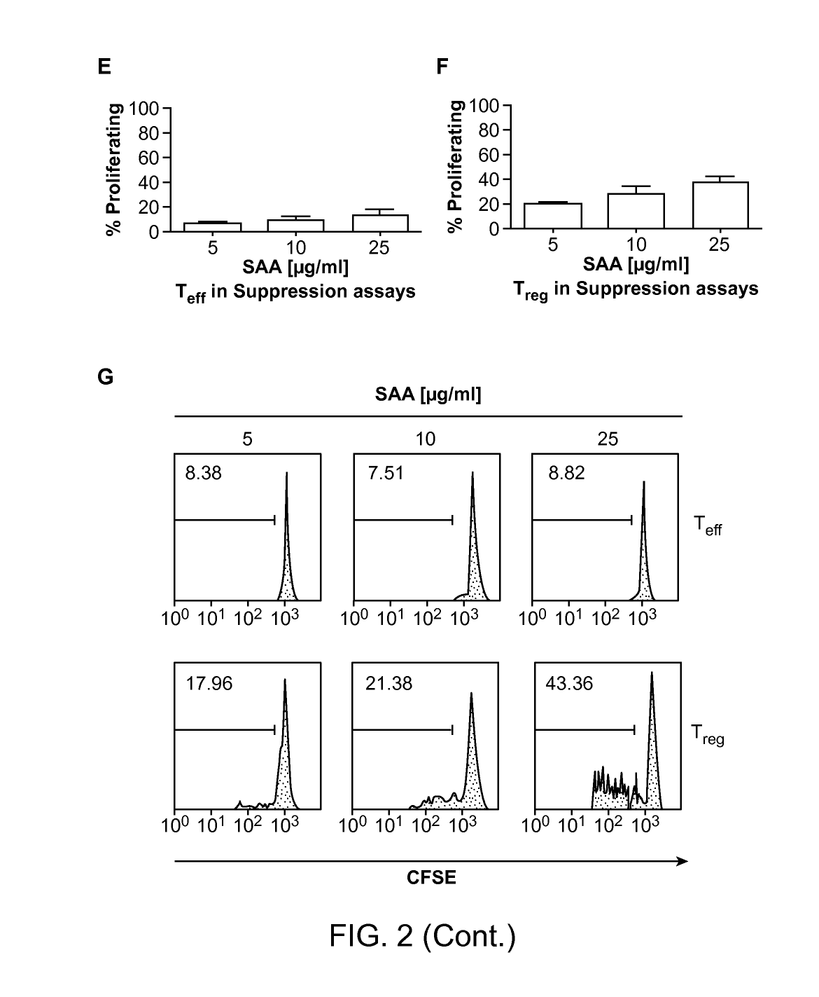

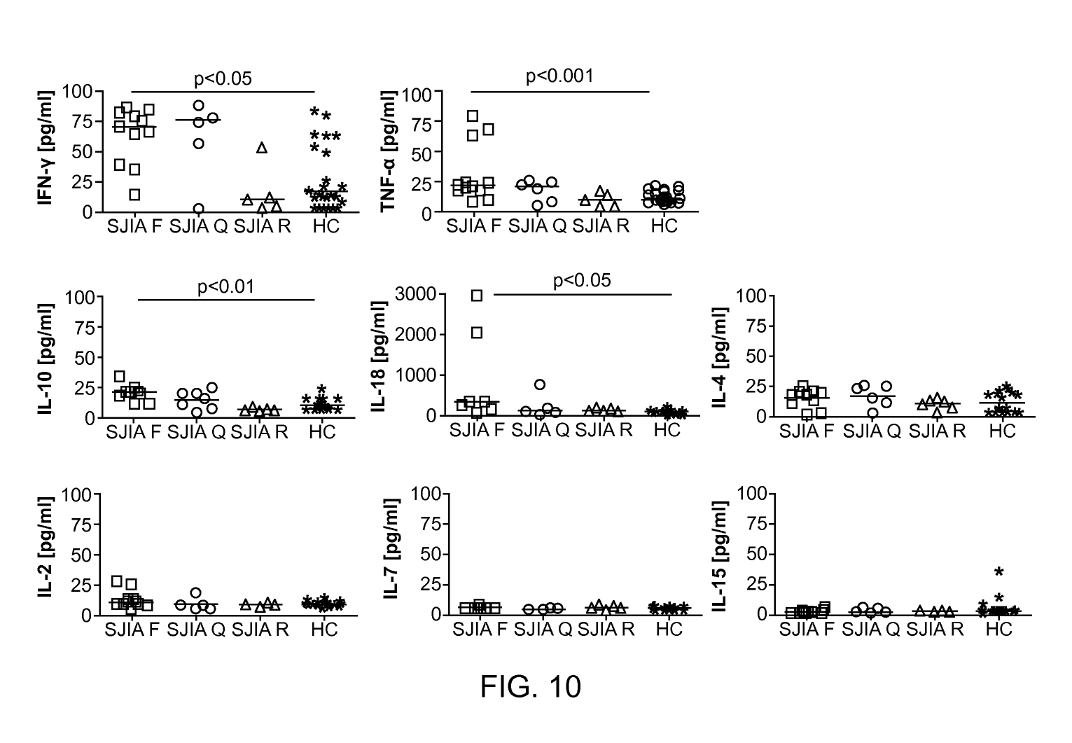

We next sought to characterize the factor(s) in SJIA plasma that induced Treg proliferation. Dialysis of plasma showed the active factor(s) was larger than 5 kD (FIG. 9D), and heat inactivation showed the plasma activity was heat-labile (FIG. 9E); these results indicated that the factor(s) was a protein. We measured the levels in SJIA plasma of various cytokines, selected based on previous data implicating them in Treg function and development or in the pathogenesis of arthritis. As expected from published work, we found significant increases in some cytokines, including TNF-α, IL-18, IFN-γ, and IL-10, in plasma samples collected at SJIA flare compared to HC plasma (FIG. 10). However, these levels fell to normal with disease remission, making them unlikely to explain the inductive effects of SJIA plasma on Treg proliferation, which were not restricted to samples from active disease. To purify the functional activity in SJIA plasma, we passed plasma samples over Cibacron Blue resin, which binds albumin, some interferons, α2-macroglobulin, coagulation factors, nucleotide-requiring enzymes, and nucleic acid-binding proteins. The column-depleted fraction became enriched for functional activity in our assay, while the eluate was virtually inactive (FIG. 9F). After size-fractionation of the active fraction (Cibacron Blue flow-through) of SJIA plasma by FPLC, the maximal functional activity was found in the fraction containing proteins smaller than 15 kD (FIG. 9G). Ion exchange chromatography indicated that the factor has low pl and acidification of HC plasma revealed activity, indicating the factor was bound to another plasma protein(s) pre-treatment. Taken together, these results brought our attention to serum amyloid A (SAA, 11.7 kD), an acute phase reactant that is a marker of SJIA-associated inflammation (Miyamae, T. et al., Arthritis Res. Ther. 2005, 7, R746-55; Elliott, M. J. et al., Br. J. Rheumatol. 1997, 36, 589-593; Booth, D. R., Booth, S. E., Gillmore, J. D., Hawkins, P. N. & Pepys, M. B., Amyloid. 1998, 5, 262-265) and is known to circulate in healthy plasma as a minor component of high density lipoprotein particles (Cunnane, G., Curr. Opin. Rheumatol. 2001, 13, 67-73; Urieli-Shoval, S., Linke, R. P. & Matzner, Y., Curr. Opin. Hematol. 2000, 7, 64-69). SJIA plasma samples from all disease stages had significantly higher levels of SAA than samples from healthy controls (FIG. 2A). To determine whether elevated expression of SAA in SJIA plasma was necessary for the induction of Treg proliferation in suppression assays, we depleted SAA from SJIA plasma with an anti-SAA antibody (FIG. 9H). Depletion of SJIA plasma with SAA-specific, but not control antibody (anti-HLA-DR), abrogated stimulation of Treg proliferation (FIG. 2B-D). Altogether, these results indicated SAA as the factor in SJIA plasma required for the selective induction of Treg proliferation in in vitro suppression assays.

Recombinant Human SAA Induces Treg Proliferation In Vitro and In Vivo.

To determine whether SAA is sufficient to reverse Treg anergy, recombinant human SAA was added to suppression assays, in the presence of polymixin to inhibit any contaminating LPS. Recombinant SAA was able to selectively enhance proliferation of Treg without reducing their suppressive activity (FIG. 2E-G). To explore the in vivo impact of SAA on Treg homeostasis, we administered recombinant human SAA to C57B6 mice by intraperitoneal injection. There was a significant increase in Treg abundance in the peritoneal cavity of SAA-injected mice compared to those injected with a control protein, purified human serum albumin, or endotoxin at the level found in recombinant human SAA (0.25 ng/ml) (FIGS. 3A, D). Furthermore, a significantly higher percentage of peritoneal Treg from SAA-injected mice expressed the nuclear antigen Ki-67, indicating that they were undergoing cell division (FIGS. 3B, D). In contrast, SAA injection did not enhance Teff proliferation in vivo (FIGS. 3C, D).

Cells are Necessary for the Induction of Treg Proliferation Mediated by SAA.

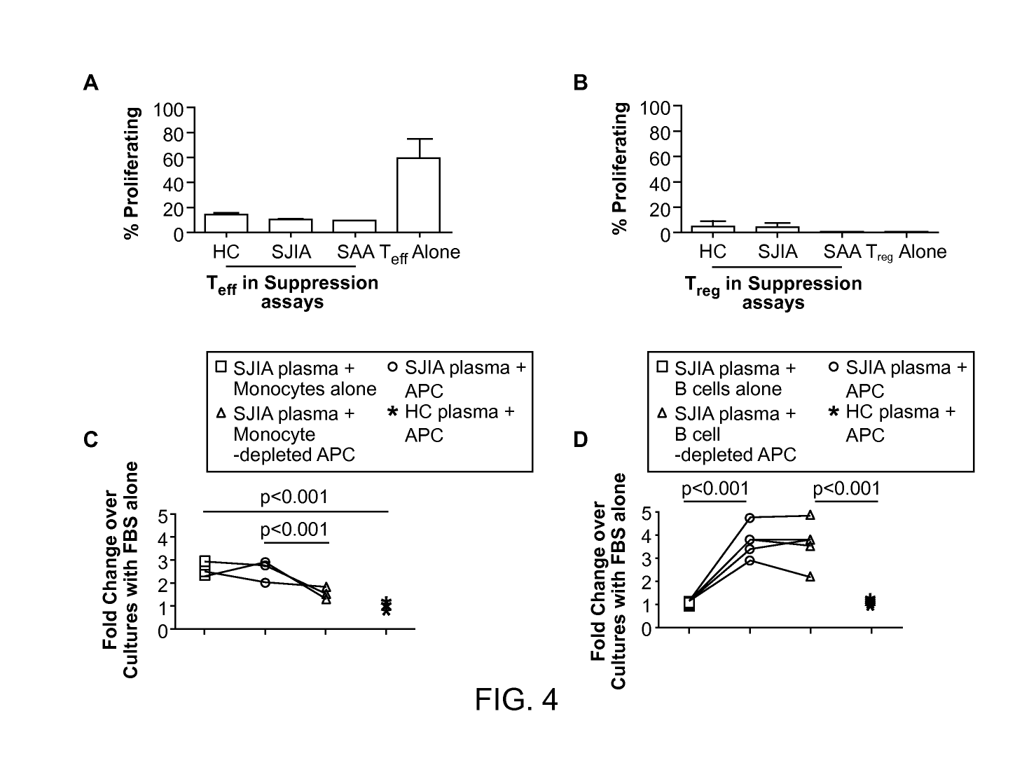

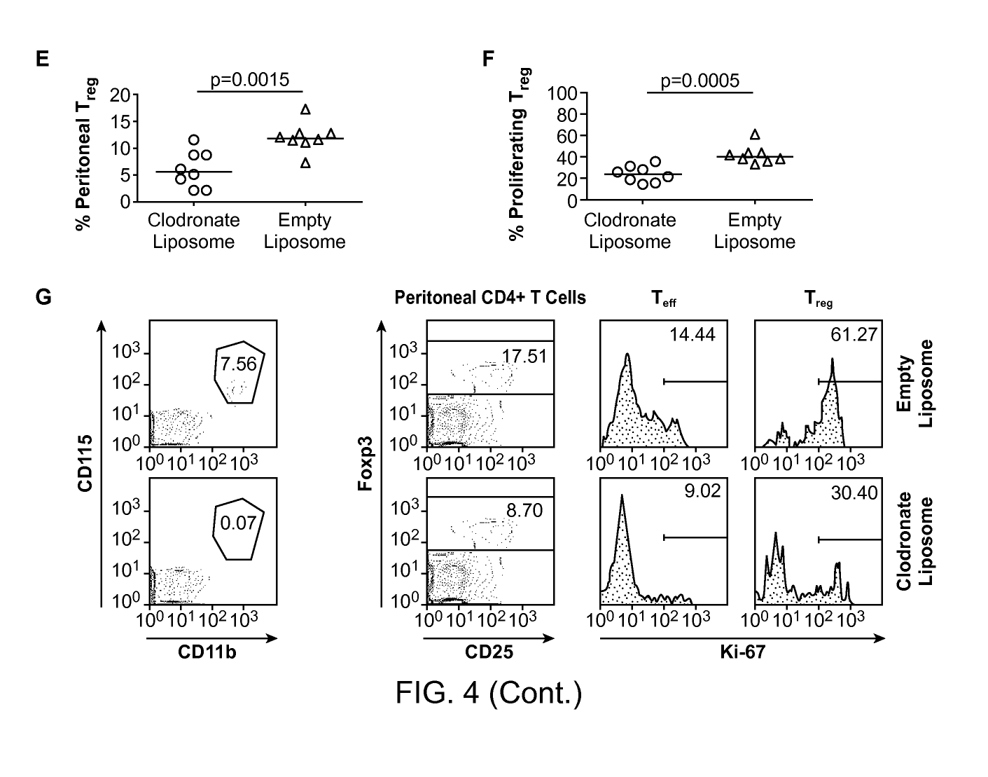

To explore the possibility that SAA might act on other cell types, such as APC, to indirectly induce Treg proliferation, we first performed suppression assays with anti-CD3 and anti-CD28 antibodies as T cell stimuli in the absence of APC. Under this condition, both SJIA plasma-derived and exogenous recombinant human SAA failed to induce Treg proliferation (FIGS. 4A, B), indicating that cells in the APC mixture mediated the mitogenic effect of SAA on Treg. To test which APC types were required for the induction of Treg proliferation, we depleted B cells or monocytes, the two more abundant professional APC subsets in peripheral blood. Depleting monocytes led to a significant decrease in cell proliferation in suppression assays with SJIA plasma (FIG. 4C). Conversely, using purified monocytes as APC in suppression assays with SJIA plasma was sufficient to induce cell proliferation at similar levels to those observed using unfractionated APC (FIG. 4C). In contrast, depletion of B cells failed to abrogate the increased proliferation in suppression assays with SJIA plasma (FIG. 4D). B cells alone were also not able to enhance cell proliferation in suppression assays with SJIA plasma (FIG. 4D). To further investigate the role of monocytes on SAA-driven Treg proliferation, we depleted monocytic cells in vivo with clodronate liposomes. Intraperitoneal injection of clodronate liposomes led to systemic depletion of monocytic cells (FIG. 4G). In the absence of monocytic cells, there were significant decreases in Treg abundance and expression of Ki-67 in the peritoneal cavities of SAA-treated animals (FIGS. 4E, F). Altogether, these results indicated that monocytes were indispensable for the mitogenic effects of SAA on Treg.

SAA Stimulates Cytokine Production by Monocytes.

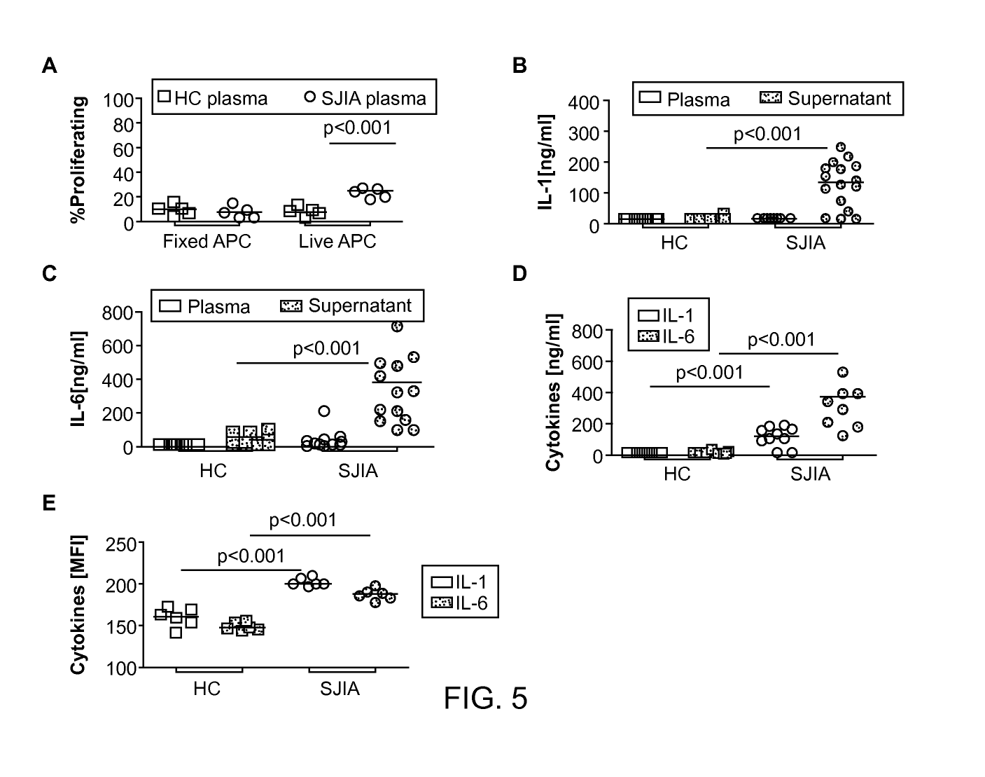

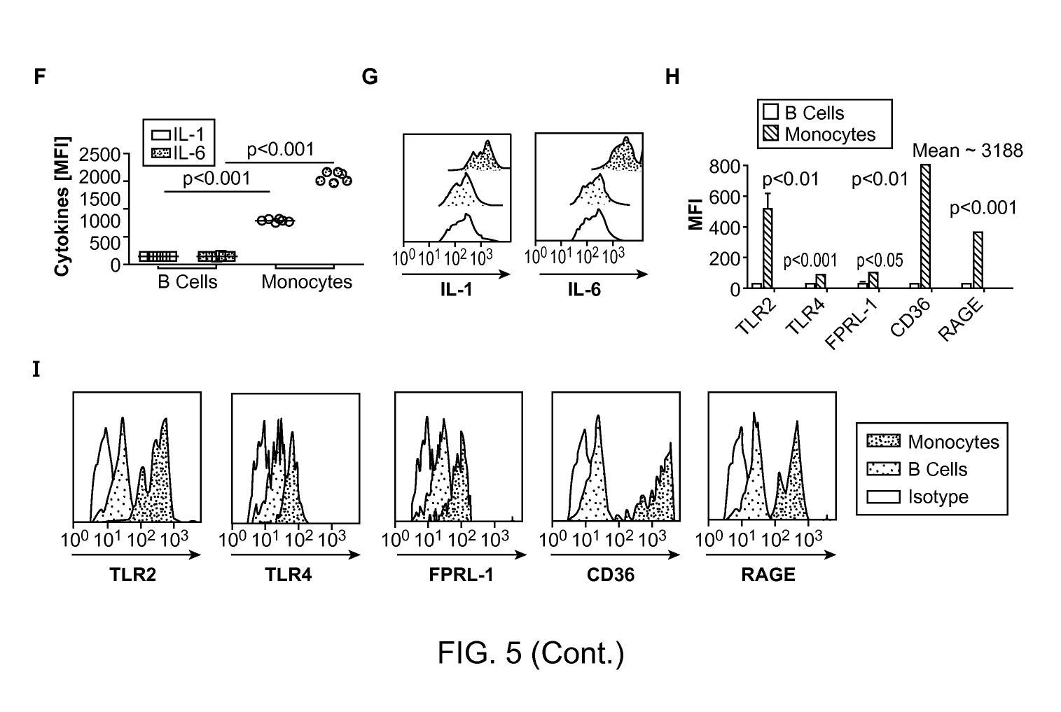

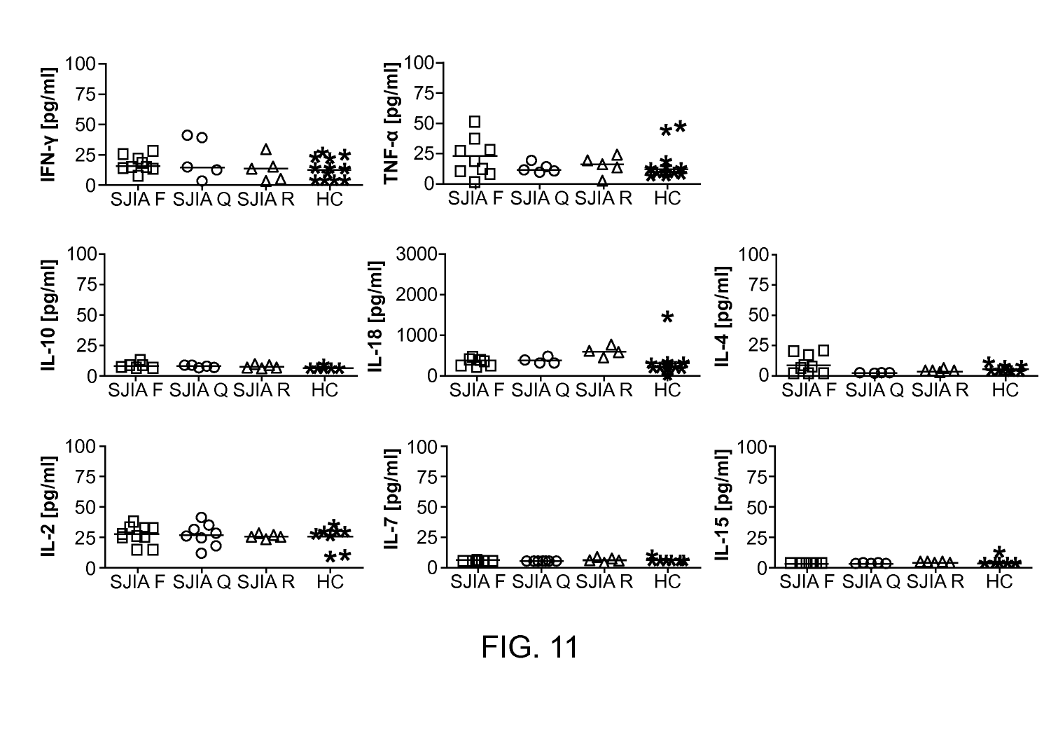

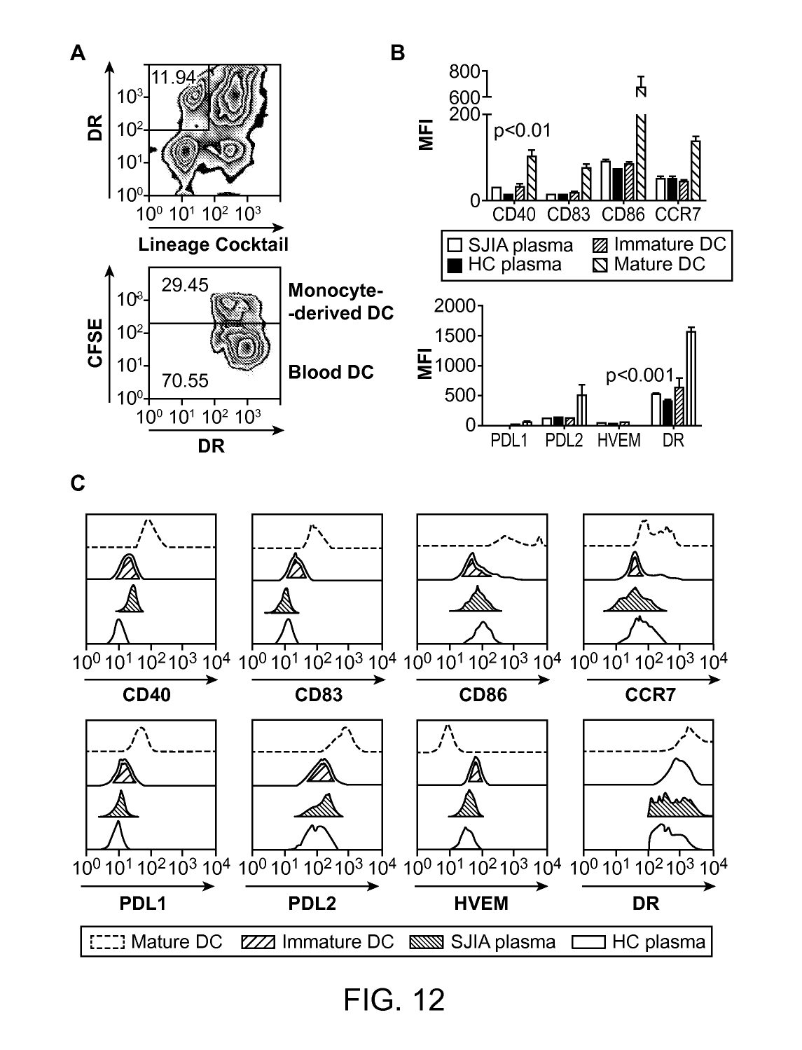

Fixation of the APC blocked their ability to support Treg proliferation in the presence of SJIA plasma in suppression assays (FIG. 5A), indicating that APC/monocyte derived soluble factors were involved in the reversal of Treg anergy. Multiplex analysis of supernatants from suppression assays showed similar levels of several cytokines, such as IFN-γ, TNF-α, IL-10, IL-18, IL-4, IL-2, IL-7, and IL-15, in cultures with SJIA plasma and HC plasma (FIG. 11). Interestingly, levels of two pro-inflammatory cytokines, IL-1 and IL-6, were significantly elevated in suppression assays with SJIA plasma compared to assays with HC plasma after 24 hours in culture (FIGS. 5B, C). No significant differences in IL-1 and IL-6 were found between SJIA plasma and HC plasma samples used in suppression assays (FIGS. 5B, C), indicating that these cytokines were actively produced by cells in suppression assays. Indeed, SJIA plasma-treated APC or monocytes, in particular, produced significantly higher levels of IL-1 and IL-6, compared to cells that were exposed to HC plasma (FIGS. 5D, E). Furthermore, it is known that SAA interacts with at least 6 distinct receptors: FPRL-1, CD36, RAGE, TLR2, TLR4, and Tanis (Baranova, I. N. et al., J. Biol. Chem. 2005, 280, 8031-8040; Cheng, N., He, R., Tian, J., Ye, P. P. & Ye, R. D., J. Immunol. 2008, 181, 22-26; Sandri, S. et al., J. Leukoc. Biol. 2008, 83, 1174-1180; Okamoto, H., Katagiri, Y., Kiire, A., Momohara, S. & Kamatani, N., J. Rheumatol. 2008, 35, 752-756; Shim, J. W. et al., Exp. Mol. Med. 2009, 41, 584-591; Su, S. B. et al., J. Exp. Med. 1999, 189:395-402; Xu, L. et al., J. Immunol. 1995, 155, 1184-1190). We found that monocytes expressed significantly higher levels of SAA receptors than B cells and produced significantly higher levels of IL-1 and IL-6 upon stimulation with recombinant SAA (FIG. 5 F-I). We also observed the emergence of a dendritic cell population with a unique phenotype in suppression assays with SJIA plasma. To track possible differentiation f CD14+ monocytes into myeloid dendritic cells (DC), CD14+ monocytes were isolated from APC, labeled with CFSE and added back to the APC mixture. In the harvested population of APC, we found two subsets of DC (Lineage− DR+ cells): one that did not stain for CFSE (blood DC from initial APC) and another that was positive for CFSE (monocyte-derived DC) (FIG. 12A). These results indicated that SJIA plasma induced monocyte differentiation into DC. We found significant increases of DR and CD40 on DC in assays with SJIA plasma compared to HC plasma after 96 hours in culture (FIGS. 12B, C). The expression of other markers, such as positive costimulatory molecules CD86, maturation markers CD83 and CCR7, inhibitory costimulatory molecules HVEM, PD-L1, and PD-L2, was not significantly different on DC in suppression assays with SJIA plasma compared to HC plasma (FIGS. 12B, C). This surface phenotype of SJIA plasma-induced DC most resembled that of immature DC (FIG. 12B-C).

Cytokines were Necessary for the Reversal of Treg Anergy.

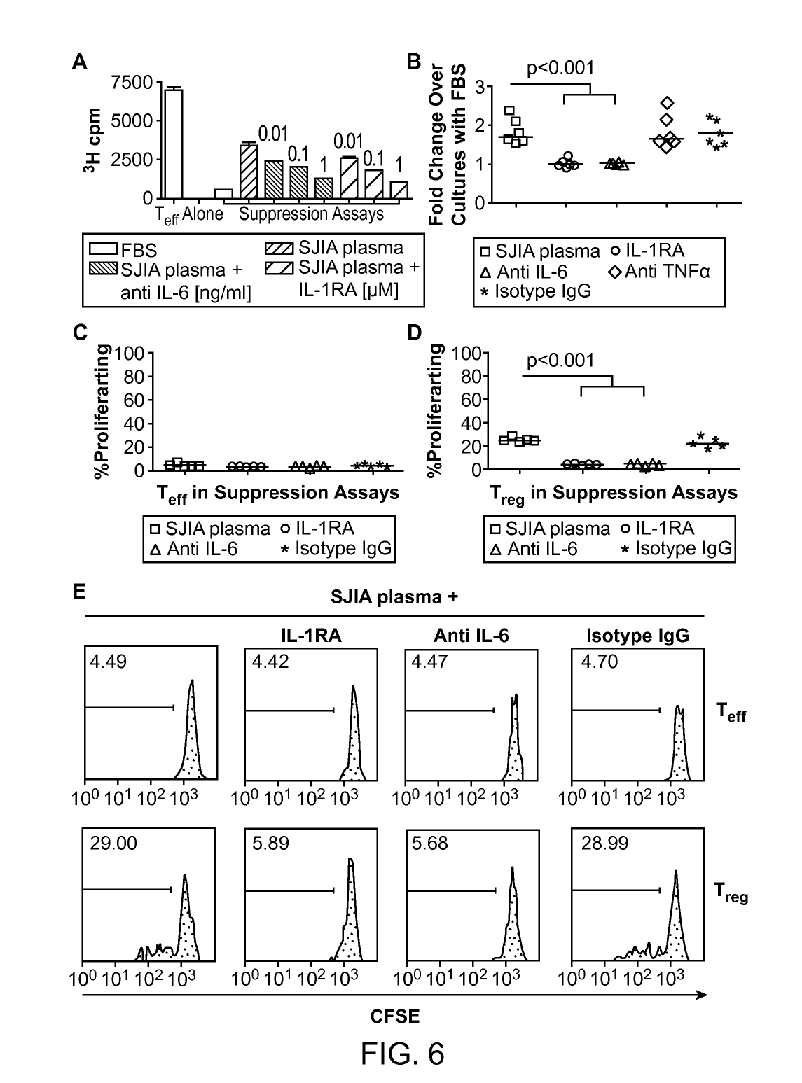

To test whether IL-1 and IL-6 produced by SAA-stimulated monocytes were required for the reversal of Treg anergy in our assays, we performed suppression assays with SJIA plasma in the presence of IL-1 receptor antagonist (IL-1Ra) or blocking antibody against IL-6 at various concentrations. Each of these reagents mediated significant inhibition of SJIA plasma-induced Treg proliferation (FIG. 6A-E). Interestingly, blocking other inflammatory cytokines, such as TNF-α, did not significantly alter SJIA plasma effects on cell proliferation (FIG. 6B). These data indicated indispensable roles for IL-1 and IL-6 in the reversal of Treg anergy by SJIA plasma in vitro, but did not rule out that other cytokines, growth factors, or cell-cell contact also contributed.

Cytokines have Distinct Effects on Mitogenic Pathways in Treg.

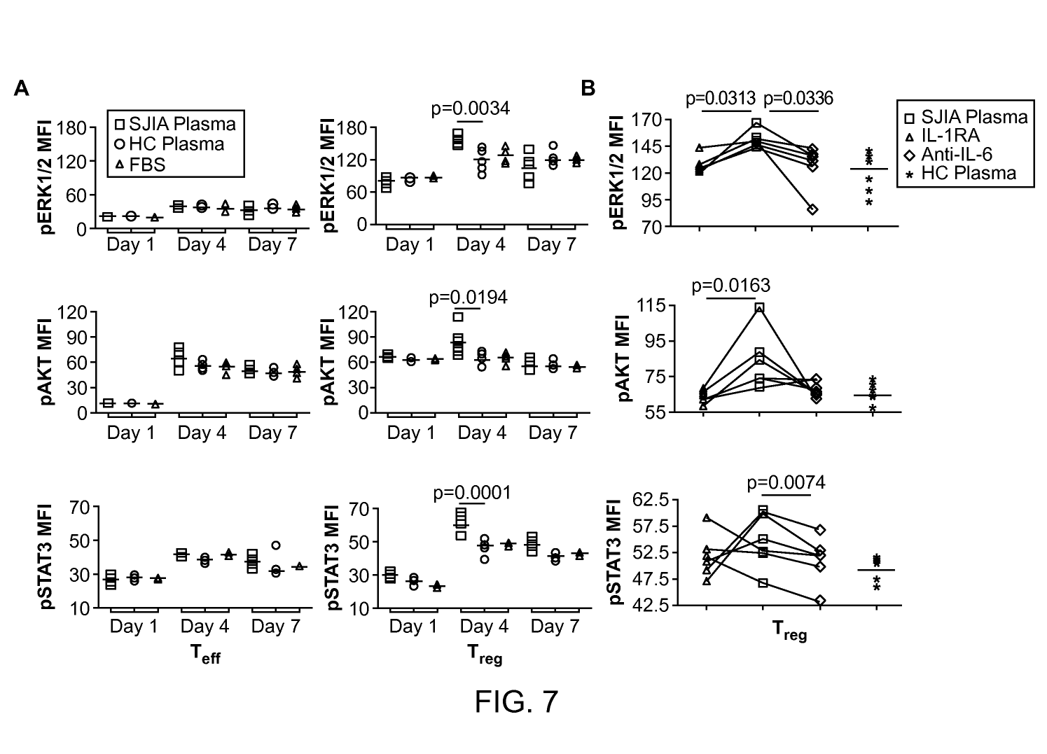

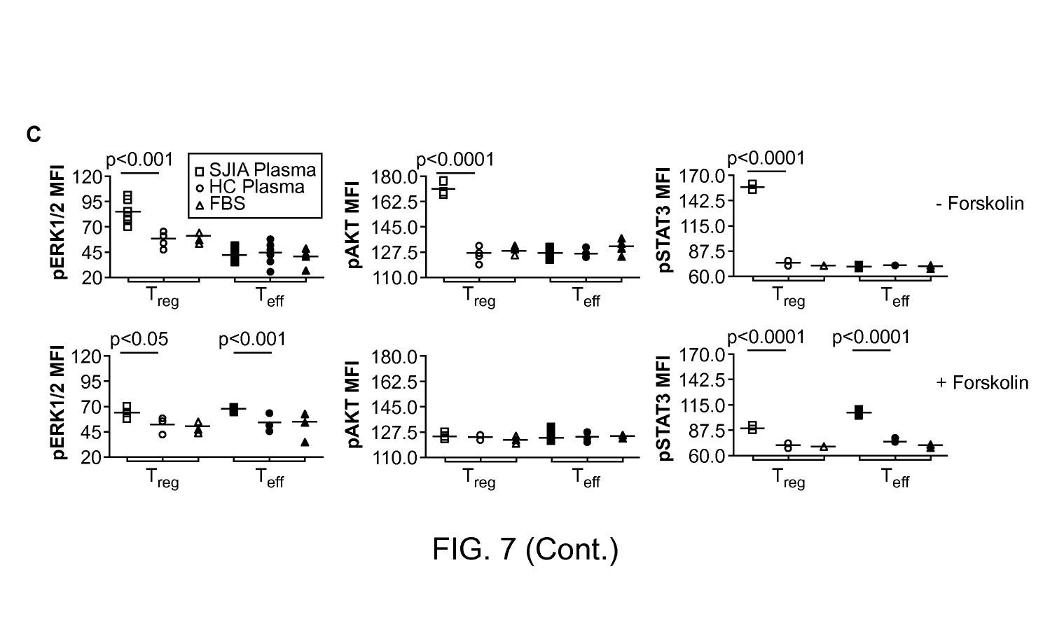

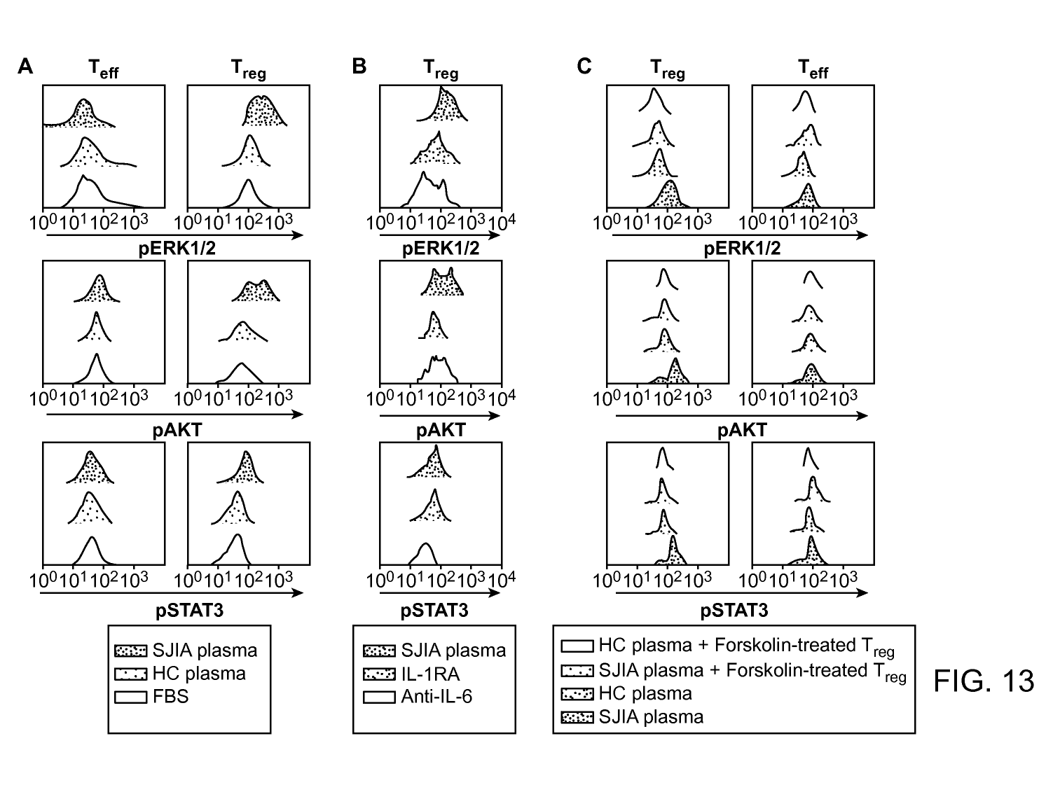

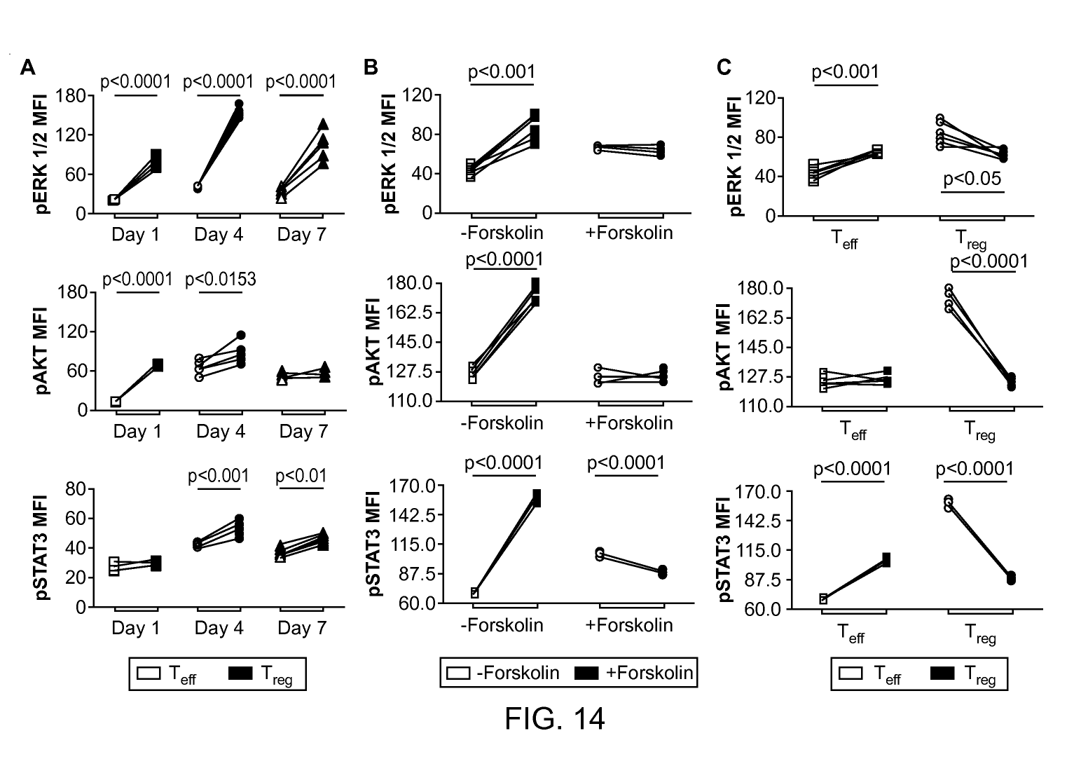

We next examined the activation state of signaling molecules that have been implicated in mitogenic processes, such as AKT and ERK1/2. We found that Treg selectively exhibited increased activation of AKT and ERK1/2 in assays with SJIA plasma compared to assays with HC plasma at day 4 (FIG. 7A, FIG. 13A). In addition, phosphorylated STAT3, a signaling molecule downstream of IL-6, was also expressed at higher levels in Treg, but not in Teff, at day 4 in suppression cultures with SJIA plasma compared to HC plasma (FIG. 7A, FIG. 13A). Treg also expressed significantly higher levels of phosphorylated AKT, ERK1/2, and STAT3 than Teff in the same suppression cultures with SJIA plasma (FIG. 14A). As IL-1 and IL-6 are mediators of Treg proliferation in these suppression cultures, we sought to link these cytokines to the activated mitogenic pathways in proliferating Treg. We analyzed activation of these pathways in the presence of IL-1 receptor antagonist (IL-1 RA) and neutralizing antibody against IL-6 in assays with SJIA plasma. We found that blocking IL-1 or IL-6 abrogated the increased expression of phosphorylated ERK1/2 in Treg at day 4 (FIG. 7B, FIG. 13B). Interestingly, blocking IL-1, but not IL-6, inhibited phosphorylation of AKT in Treg at day 4 (FIG. 7B, FIG. 13B). Blocking IL-6 also abrogated STAT3 activation in Treg at day 4 (FIG. 7B, FIG. 13B). Thus, these results indicated that IL-1 and IL-6 were necessary for the activation of mitogenic pathways in Treg and Teff and also revealed differential activation of these pathways by these cytokines.

Treg Exhibit Lower Expression of Mitogenic Signaling Pathways Compared to Teff.

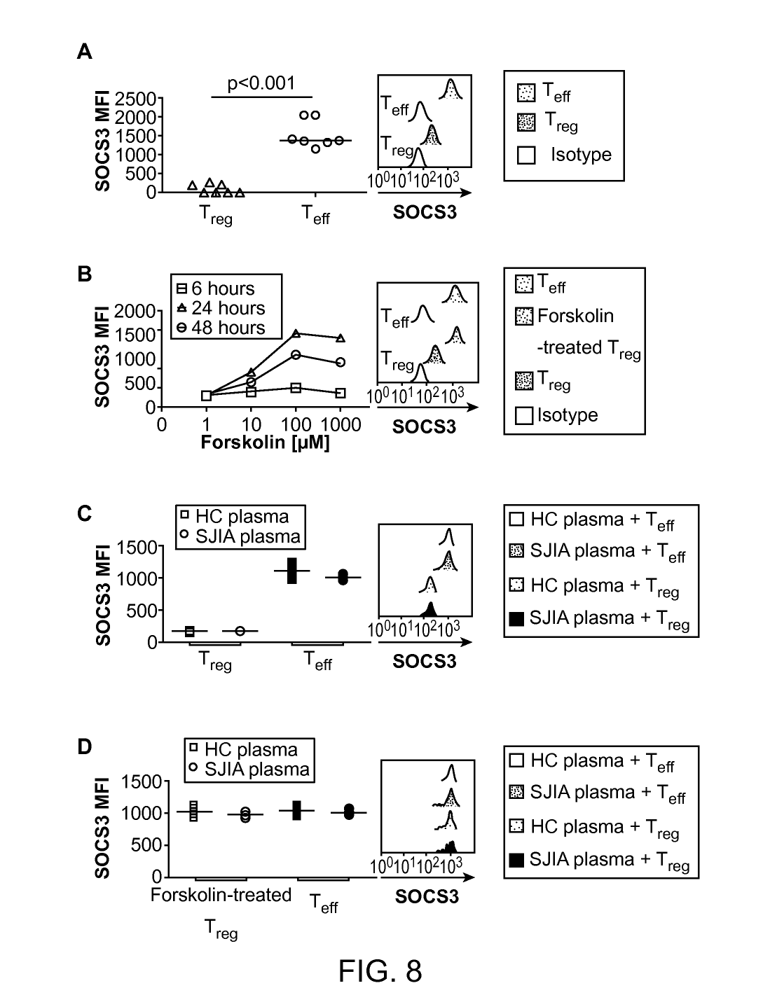

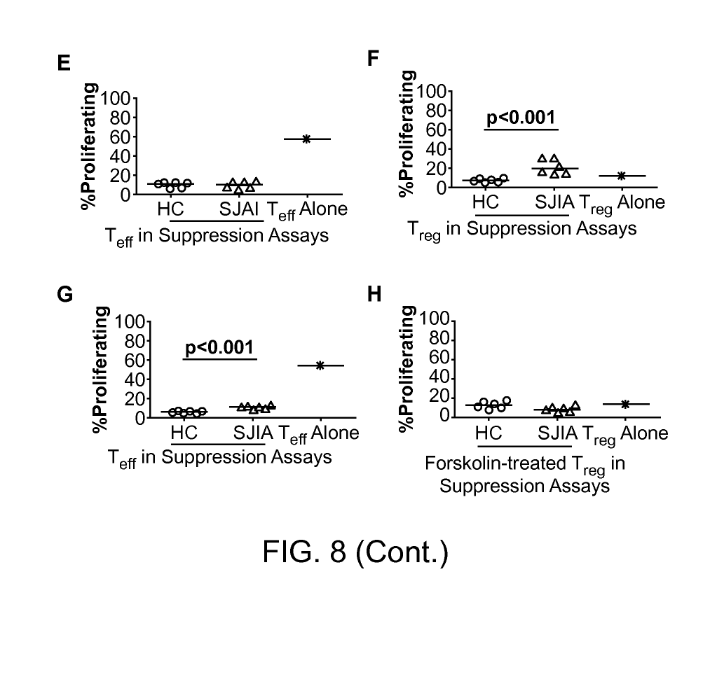

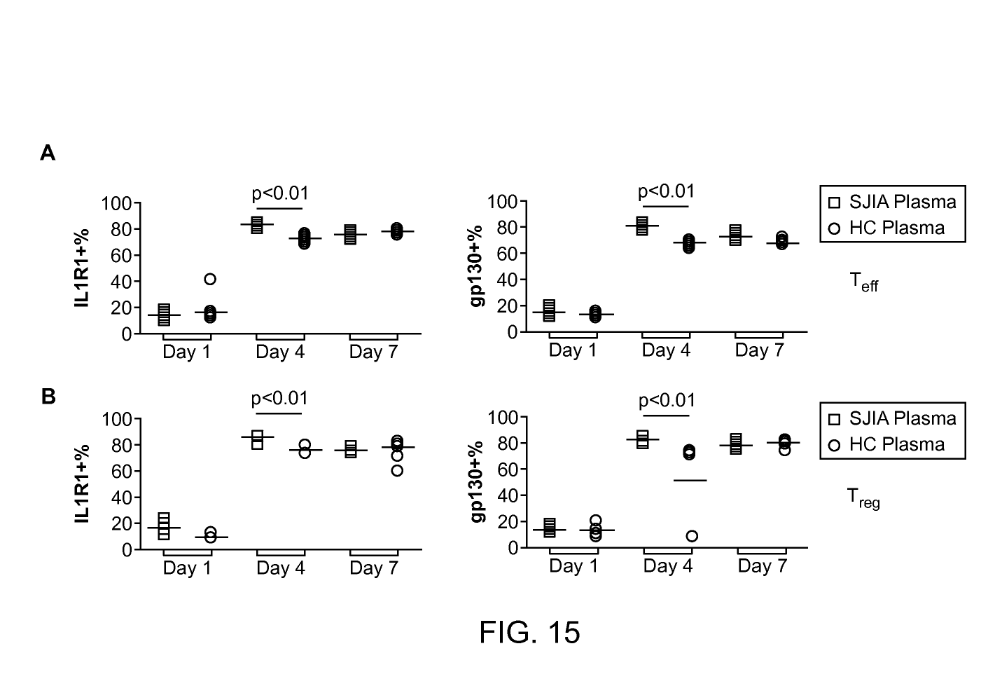

To investigate the mechanism that is responsible for the selective activation of mitogenic signaling pathways in response to IL-1 and IL-6 by Treg, we first measured the expression of surface receptors for these two cytokines on Treg and Teff. We found that IL-1R1 (the high affinity IL-1 receptor) and the signaling component of IL-6 receptor complex (gp130) were expressed at low levels in Treg and Teff after 24 hours in suppression assays (FIG. 15). However, expression of these molecules on Treg and Teff was up-regulated during the course of the assay (FIG. 15). Furthermore, higher expression of IL-1 and IL-6 receptors on both Treg and Teff was induced after 96 hours in the presence of SJIA plasma compared to HC plasma (FIG. 15), despite differential responsiveness of these cell types to these cytokines. A possible explanation for these seemingly paradoxical results was that Treg and Teff differed in regulatory mechanisms that control IL-1 and IL-6 intracellular signaling. Indeed, we found that SOCS3, a suppressor of cytokine signaling proteins and a chief regulator of both IL-1 and IL-6 signaling (Wong, P. K. et al., J. Clin. Invest. 2006, 116, 1571-1581; Lang, R. et al., Nat. Immunol. 2003, 4, 546-550; Frøbose, H. et al., Mol. Endocrinol. 2006, 20, 1587-1596), was expressed at significantly lower levels in Treg compared to Teff (FIG. 8A).

Induction of SOCS3 in Treg Inhibits their Selective Proliferation Driven by SJIA Plasma.

To test the hypothesis that the difference in SOC3 expression in Treg and Teff regulates their selective response to IL-1 and IL-6, we modulated SOCS3 expression in Treg using forskolin, a reagent known to induce SOCS3 expression in various cell types (Gasperini, S. et al., Eur. Cytokine Netw. 2002, 13, 47-53; Barclay, J. L., Anderson, S. T., Waters, M. J. & Curlewis, J. D., Mol. Endocrinol. 2007, 21, 2516-2528; St-Onge, M. et al., PLoS. One 2009, 4, e4902). Freshly isolated Treg were cultured with different concentrations of forskolin and their expression of SOCS3 was examined at different time points. Intracellular staining showed an increase in SOCS3 expression by forskolin-treated Treg (FIG. 8B). Optimal dosing for forskolin-mediated SOCS3 induction in Treg was determined to be 100 μM after 24 hour stimulation (FIG. 8B). This condition was then used to evaluate the proliferative response of Treg to SJIA plasma in suppression assays. SOCS3 expression by Treg, initially induced by forskolin, remained at a level comparable to that in Teff during the suppression assays (FIG. 8C-D). Forskolin-treated Treg still suppressed Teff proliferation, but they no longer proliferated in response to inflammatory signals in SJIA plasma (FIG. 8E-H). Similar results were obtained with SOCS3 transfection in Treg. Altogether, these results indicated that the level of expression of SOCS3 by Treg regulates the dynamic range of their proliferative response to SJIA plasma in suppression assays.

Induction of SOCS3 in Treg Abrogates their Selective Activation of Mitogenic Signaling Driven by SJIA Plasma.

We predicted SOCS3 up-regulation would alter activation of mitogenic signaling molecules by Treg. In assays with forskolin-treated Treg, levels of pERK1/2 and pSTAT3 in Treg were significantly reduced, and activation of AKT was abrogated at day 4 (FIG. 7C, FIG. 13C). Increased activation of mitogenic signaling in Treg compared to Teff co-cultured in the same assays with SJIA plasma was either abrogated (for ERK1/2 and AKT) or reversed (Teff became more activated with respect to STAT3) when forskolin-treated Treg were used (FIG. 14B). Forskolin-treated Treg also showed a significant decrease in activation of ERK1/2, AKT, and STAT3 compared to untreated Treg that were exposed to the same SJIA plasma in suppression assays (FIG. 14C). Conversely, in the presence of forskolin-treated Treg, activation of ERK1/2 and STAT3 was modestly enhanced in Teff in assays with SJIA plasma compared to those with HC plasma at day 4 (FIG. 7C, FIG. 13C). Teff co-cultured with forskolin-treated Treg also showed a modest increase in ERK1/2 and STAT3 activation compared to those co-cultured with untreated Treg (FIG. 14C). Collectively, these results indicate that Treg-specific modulation of SOCS3 expression tunes the competitive fitness between Treg and Teff in response to inflammation-induced activation of mitogenic pathways.

### DISCUSSION

In human diseases that are characterized by inflammation, increased numbers of Treg have been found in inflamed tissues where local SAA production occurs (Heller E A, et al. Chemokine CXCL10 promotes atherogenesis by modulating the local balance of effector and regulatory T cells. Circulation 2006; 113(19):2301-2312; Patel S, et al. The “atheroprotective” mediators apolipoproteinA-I and Foxp3 are over-abundant in unstable carotid plaques. Int J Cardiol 2010; 145(2):183-187; Bunnag S, et al. FOXP3 expression in human kidney transplant biopsies is associated with rejection and time post transplant but not with favorable outcomes. Am J Transplant 2008; 8(7):1423-1433). SAA is elevated within the same time-frame of accumulation of Treg during tissue injury (Hillman N H, et al. Brief, large tidal volume ventilation initiates lung injury and a systemic response in fetal sheep. Am J Respir Crit. Care Med 2007; 176(6):575-581; Jurawitz M C, et al. Kinetics of regulatory T cells in the ovalbumin asthma model in the rat. Int Arch Allergy Immunol 2009; 149(1):16-24). Our in vivo data indicates that early induction of SAA at inflammatory sites generates a milieu that drives Treg proliferation. Our study also indicates that modulating the level of expression of SOCS3 in Treg and, consequentially, the relative expression of SOCS3 in Treg versus Teff by pharmacologic means abrogates the selective activation of mitogenic signaling pathways in Treg on exposure to the micro-environment generated by interaction between SAA and monocytes. These results imply that maintenance versus resolution of inflammatory processes might depend on the relative responsiveness of proinflammatory/effector and anti-inflammatory/regulatory cell subsets. Reduced SOCS3 expression in regulatory cell subsets, such as Treg, might increase their sensitivity to inflammation-derived mitogenic signals, leading to their rapid activation and effective suppression of inflammatory cell types. It is of interest that mediators that could induce SOCS3 expression, such as IL-10, are also produced by Treg and therefore might serve as negative feedback regulators of Treg proliferation to exert a fine balance on tolerance versus immunity under inflammatory conditions.

Inflammation associated with infection or tissue injury affords an opportunity for molecular mimicry or exposure of previously cryptic tissue antigens to increase the risk of autoimmunity. These possibilities highlight the need for timely recruitment and/or expansion of tolerogenic cell subsets at inflammatory sites. The mitogenic effects of SAA on Treg shown here represents a mechanism for protection against the potential breach of tolerance unleashed by inflammation.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method for preparing an expanded population of regulatory T cells, comprising:
contacting a leukocyte population comprising regulatory T cells and antigen presenting cells with an effective amount of a serum amyloid A (SAA) composition to induce regulatory T cell proliferation.

2. The method according to claim 1, wherein the SAA composition is selected from a SAA polypeptide or fragment thereof and a polynucleotide encoding a SAA polypeptide or fragment thereof.

3. The method according to claim 1, wherein the antigen presenting cells are monocytes.

4. The method according to claim 1, wherein the regulatory T cells are CD4⁺CD25⁺ regulatory T cells.

5. The method according to claim 1, wherein the leukocyte population is contacted in vitro.

6. The method according to claim 1, wherein the leukocyte population is contacted in vivo.

7. The method according to claim 1, further comprising the step of measuring the proliferation of the regulatory T cells.

8. A method for preparing an expanded population of regulatory T cells, comprising:
contacting regulatory T cells with
i) a serum amyloid A (SAA) composition, and
ii) interleukin 1 (IL-1) and/or interleukin 6 (IL-6),
in an amount effective to induce regulatory T cell proliferation.

9. The method according to claim 8, wherein the SAA composition is selected from a SAA polypeptide or fragment thereof and a polynucleotide encoding a SAA polypeptide or fragment thereof.

10. The method according to claim 8, wherein the regulatory T cells are CD4⁺CD25⁺ regulatory T cells.

11. The method according to claim 8, wherein the regulatory T cells are contacted in vitro.

12. The method according to claim 8, wherein regulatory T cells are contacted in vivo.

13. The method according to claim 8, further comprising the step of measuring the proliferation of the regulatory T cells.

14. A method for treating autoimmune or rheumatoid disease, the method comprising:
contacting the regulatory T cells of a subject having an autoimmune disease or rheumatoid disease with an effective amount of a SAA composition to treat the autoimmune disease or rheumatoid disease.

15. The method according to claim 14, wherein the SAA composition is selected from a SAA polypeptide or fragment thereof and a polynucleotide encoding a SAA polypeptide or fragment thereof.

16. The method according to claim 14, wherein the method further comprises contacting the regulatory T cells of the subject with an effective amount of interleukin 1 (IL-1) and/or interleukin 6 (IL-6).

17. The method according to claim 14, wherein the method further comprises measuring the proliferation of the regulatory T cells.

18. The method according to claim 14, wherein the method further comprises measuring the treatment of the autoimmune disease in the subject.

19. The method according to claim 14, wherein the autoimmune disease is rheumatoid arthritis, juvenile idiopathic arthritis, systemic lupus erythematosus, spondyloarthropathy, psoriatic arthritis, Kawasaki disease, Sjogren's syndrome, sarcoidosis, multiple sclerosis, type 1 diabetes mellitus, graft versus host disease, transplant rejection, ulcerative colitis or Crohn's disease.

20-24. (canceled)

25. The method according to claim 14, wherein the contacting comprises administering the SAA composition to the subject.

26. The method according to claim 14, wherein the contacting comprises contacting the regulatory T cells with the SAA composition ex vivo, and transplanting the contacted regulatory T cells into the individual.


SEQUENCE LISTING (10 sequences)

SEQ ID NO: 1 - DNA, 678 bp, Homo sapiens
ggcagggacccgcagctcagctacagcacagatcaggtgaggagcacaccaaggagtgatttttaaaacttactctgttttctctttcccaacaagattatcatttcctttaaaaaaaatagttatcctggggcatacagccataccattctgaaggtgtcttatctcctctgatctagagagcaccatgaagcttctcacgggcctggttttctgctccttggtcctgggtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggctgcagaagtgatcagcgatgccagagagaatatccagagattctttggccatggtgcggaggactcgctggctgatcaggctgccaatgaatggggcaggagtggcaaagaccccaatcacttccgacctgctggcctgcctgagaaatactgagcttcctcttcactctgctctcaggagatctggctgtgaggccctcagggcagggatacaaagcggggagagggtacacaatgggtatctaataaatacttaagaggtggaatttgtggaaa

SEQ ID NO: 2 - protein, 122 aa, Homo sapiens
MKLLTGLVFCSLVLGVSSRSFFSFLGEAFDGARDMWRAYSDMREANYIGSDKYFHARGNYDAAKRGPGGAWAAEVISDARENIQRFFGHGAEDSLADQAANEWGRSGKDPNHFRPAGLPEKY

SEQ ID NO: 3 - DNA, 531 bp, Homo sapiens
ggcagggacccgcagctcagctacagcacagatcagcaccatgaagcttctcacgggcctggttttctgctccttggtcctgggtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggctgcagaagtgatcagcgatgccagagagaatatccagagattctttggccatggtgcggaggactcgctggctgatcaggctgccaatgaatggggcaggagtggcaaagaccccaatcacttccgacctgctggcctgcctgagaaatactgagcttcctcttcactctgctctcaggagatctggctgtgaggccctcagggcagggatacaaagcggggagagggtacacaatgggtatctaataaatacttaagaggtggaatttgtggaaa

SEQ ID NO: 4 - protein, 122 aa, Homo sapiens; identical to SEQ ID NO: 2

SEQ ID NO: 5 - DNA, 592 bp, Homo sapiens
ggcagggacccgcagctcagctacagcacagatcagttatcctggggcatacagccataccattctgaaggtgtcttatctcctctgatctagagagcaccatgaagcttctcacgggcctggttttctgctccttggtcctgggtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggctgcagaagtgatcagcgatgccagagagaatatccagagattctttggccatggtgcggaggactcgctggctgatcaggctgccaatgaatggggcaggagtggcaaagaccccaatcacttccgacctgctggcctgcctgagaaatactgagcttcctcttcactctgctctcaggagatctggctgtgaggccctcagggcagggatacaaagcggggagagggtacacaatgggtatctaataaatacttaagaggtggaatttgtggaaa

SEQ ID NO: 6 - protein, 122 aa, Homo sapiens; identical to SEQ ID NO: 2

SEQ ID NO: 7 - DNA, 594 bp, Homo sapiens
aggctcactataaatagcagccacctctccctggcagacagggacccgcagctcagctacagcacagatcagcaccatgaagcttctcacgggcctggttttctgctccttggtcctgagtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggctgcagaagtgatcagcaatgccagagagaatatccagagactcacaggccgtggtgcggaggactcgctggccgatcaggctgccaataaatggggcaggagtggcagagaccccaatcacttccgacctgctggcctgcctgagaaatactgagcttcctcttcactctgctctcaggagacctggctatgaggccctcggggcagggatacaaagttagtgaggtctatgtccagagaagctgagatatggcatataataggcatctaataaatgcttaagaggtggaatttgttgaaaca

SEQ ID NO: 8 - protein, 122 aa, Homo sapiens
MKLLTGLVFCSLVLSVSSRSFFSFLGEAFDGARDMWRAYSDMREANYIGSDKYFHARGNYDAAKRGPGGAWAAEVISNARENIQRLTGRGAEDSLADQAANKWGRSGRDPNHFRPAGLPEKY

SEQ ID NO: 9 - DNA, 2053 bp, Homo sapiens
aggctcactataaatagcagccacctctccctggcagacagggacccgcagctcagctacagcacagatcagcaccatgaagcttctcacgggcctggttttctgctccttggtcctgagtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggctgcagaagtgatcagtttattttcagctgaactgtagagagtgaaggaagaagccttttttcttcactcctacatgacccaggcattgatccaggcaatgaagattcagtgtaaataaccaccactaacaagaccatggcctttggaacctgtgctaagaggcatggatgagctccctcagcatgtggatggagactgaagagaggtctgaaggctcagtgtggtgtctccattctctaagaagtttggaggagaagctggatacagcaaaacagactgagaaggagcagctgttgggaaaggaggaaaactaggaaagcatggtgttccagaatccctgatggagatattgattagtagtatcagattctgcttagcagttgaggagttcacctttggcttaagcaacatggagtcattgactatctcgtgaaggtggttgcaggagaaggtctggagaactaaatggagcagtgataagagagaatgggagatggtatgataggactcctggacaccccagacatcaatcaaaacaccacagacaagaaggtgtggatacaaaaactagcagttagaagaaataatagaaaatgaactccaactacttctgaaaaaaaagtaatgagggcaattaatacattgaagaaagcctcaataagtacaacagtgtagtactgacttatatatagaaaaaacctgatcagtggaaccaaatcctcagaattagacataagtgcatatgagaattttgtatgtgataaaggttgtatgttaacgtattaagtaaaagacggactattcaacaaatgggattggtacaactgggtgaccatctaaaataacatcatgttgaaaacatactgtatatattacggtaggacaaattccaaatatgctaaatatttataaacaaataaggaaaatgataataataatattaacattatctggtgaatccatggaaaattcttttatagcccaaagtagggaaagctgcaacaacaaggatggaactggaggtcattatgctaagtgaaatcattcaggaacagaaagacaaacatcacatgttctcacttatttgtgggatctaaaaatcaaaacaattgaattcatggagatagagaatagaaggatggttaccagaggctgggaagggtagtggggagggggggagtgaggggaggtgaggatggttaatgagtaccaaaaaaatacttagaatgaataagagctagtatttgatagcacaacgggggactatagtcagtaataatttagctgtacattgtaaaataaccaaaagagtataattggattatttgtaacacaaaggataaatgcttgaggggatggatacccaattttccatgatgtgattattgcgcattgcatggctgtaccaaaatatctcatgtaccccataattatatacacctactatgtacccacaaaaaatttttaaaaaggaatgaaataaaaacatttgagtttaagaaaaccacaaaaaaacaaagtagggaaagctcttcaaatactatgaaattgagaagacaactaaagaaaatattaataaagacaagaacataaataatttctttggcagaacataagccatcataatcagaagataaataagctgggaaaaatatttgtaactcatatcctagataacatactcattttccctgtatataaaaagtttctctaatgtgataaataaaaacacaataaaccagtaaaaaatgaaaaaaaaaaa

SEQ ID NO: 10 - protein, 83 aa, Homo sapiens
MKLLTGLVFCSLVLSVSSRSFFSFLGEAFDGARDMWRAYSDMREANYIGSDKYFHARGNYDAAKRGPGGAWAAEVISLFSAEL